(19) United States
(12) Patent Application Publication
(10) Pub. No.: US 2025/0345392 A1
(43) Pub. Date: Nov. 13, 2025

(54) ANNEXIN A1 LIQUID PHARMACEUTICAL COMPOSITION

(71) Applicant: ResoTher Pharma A/S, Holte (DK)

(72) Inventor: Thomas Engelbrecht Nordkild Jonassen, Holte (DK)

(21) Appl. No.: 18/871,530

(22) PCT Filed: Jun. 30, 2023
(86) PCT No.: PCT/EP2023/067975

§ 371 Date: Dec. 4, 2024

(30) Foreign Application Priority Data
Jun. 30, 2022 (EP) 22182341.2

(51) Int. Cl. A61K 38/17; A61K 9/00; A61K 9/08; A61K 47/18; A61K 47/26

(52) U.S. Cl. CPC A61K 38/1709; A61K 9/0019; A61K 9/08; A61K 47/183; A61K 47/26

(57) ABSTRACT

The present disclosure relates to a liquid pharmaceutical composition comprising an Annexin A1 (AnxA1) N-terminal-peptide and at least one pharmaceutically acceptable excipient for intravenous (i.v.) infusion and its use in the treatment of an ischemic condition and/or an inflammatory condition.

### TECHNICAL FIELD

The present disclosure relates to a liquid pharmaceutical composition comprising an Annexin A1 (AnxA1) N-terminal-peptide and at least one pharmaceutically acceptable excipient, wherein the liquid pharmaceutical composition is to be administered to a human subject as a 1-120 minutes intravenous (i.v.) infusion, and wherein the AnxA1 N-terminal-peptide has a terminal elimination half-life (t_{1/2}) of at least 30 minutes after said i.v. infusion. The present disclosure further relates to said liquid pharmaceutical composition for use in the treatment of an ischemic condition and/or an inflammatory condition.

### BACKGROUND

The Annexin super-family consists of 13 calcium phospholipid binding proteins with significant biological and structural homology. Annexins are structurally divided into a highly conserved core domain and a variable N-terminal domain. Annexin A1 (ANXA1, 37 kDa—SEQ ID NO: 1) is an anti-inflammatory protein that inhibits extravasation of blood-borne polymorphonuclear leukocyte (PMN) into the surrounding tissue. The protein binds to the N-formyl peptide receptor (FPR) 2 or FPR-L1 receptor, where it initiates a cascade of signaling events. Following an inflammatory stimulus, migration of blood-borne polymorphonuclear leukocyte (PMN) into the surrounding tissue takes place. Transmigration or extravasation of PMN is regulated by mediators such as adhesion molecules, cytokines and proteases, which control the pro-inflammatory and anti-inflammatory processes. The disruptive potential of the PMN is high and potentially self-damaging. Thus, controlling extravasation of PMN and the inflammatory response is important.

For therapeutic purposes as an anti-inflammatory agent, the full Annexin A1 protein has numerous disadvantages relative to functional fragments or modified versions thereof. The large size of the protein makes it more difficult to deliver by techniques that are possible with a smaller polypeptide (e.g. transdermally or transmucosally). For use to treat inflammation of the eyes, a smaller molecule is expected to be better able to penetrate the corneal epithelium. Also, susceptibility to proteolytic degradation is a particular concern for all peptide pharmaceuticals, especially large ones and especially if oral delivery (preferred by many patients) is contemplated.

Some Annexin A1 derivatives lacking significant regions on the N-terminal side of the polypeptide have been shown to lack significant activity in some assays of inflammation and mediator release, whereas the full length N-terminus N-acetyl Annexin A1 was deemed biologically active in several systems. A number of peptides primarily derived from the unique N-terminal portion of the Annexin A1 protein have been shown to possess anti-inflammatory properties. One of the most extensively studied Annexin A1 peptides is peptide Ac2-26, which mimics the 2^{nd} to the 26^{th} amino acids of the 54-amino acid N-terminal region. It has been show that Annexin A1 and its N-terminal peptide (Ac2-26) exert the majority of their anti-inflammatory action through the FPR2/Lipoxin A4 (FPR2/Alx) receptor. In vivo the Ac2-26 peptide has been shown to exert an anti-inflammatory effect in models of myocardial ischaemia reperfusion (I/R), mesentery I/R, glycogen peritonitis and IL1 air pouch, where it was reported to significantly reduce the recruitment of neutrophils to the site of injury/inflammation.

Shorter versions of the Ac2-26 peptide, such as peptides Ac2-12 and Ac2-6, have also been shown to elicit some degree of anti-inflammatory effects in acute models of inflammation. Longer polypeptides with anti-inflammatory effects, such as polypeptides corresponding to amino acid residues 2-48, 2-50 and 11-48, have been disclosed, including protease-resistant V24L variants (WO 2012/174397 and WO 2022/038281).

Given the therapeutic potential of Annexin A1 (AnxA1) N-terminal-peptides there is a need for developing safe, effective and stable formulations that can be efficiently administered.

### SUMMARY

There is a need for developing safe, effective and stable formulations of AnxA1 N-terminal-peptides that can be administered by intravenous (i.v.) infusion to a human subject which provides a sufficiently long half-life (t_{1/2}), such as terminal elimination half-life (t_{1/2}), of the AnxA1 N-terminal-peptide. The present inventors show that a liquid formulation of an AnxA1 2-48 peptide when administered as a 30-minute i.v. infusion to a human subject display a sufficiently long half-life (t_{1/2}), which half-life is surprisingly higher in humans than in animals.

In some embodiments, the present disclosure relates to a liquid pharmaceutical composition comprising an Annexin A1 (AnxA1) N-terminal-peptide and at least one pharmaceutically acceptable excipient, wherein the liquid pharmaceutical composition is to be administered to a human subject as a 1-120 minutes intravenous (i.v.) infusion, and wherein the AnxA1 N-terminal-peptide has a terminal elimination half-life (t_{1/2}) of at least 30 minutes after i.v. infusion.

In some embodiments, the present disclosure relates to liquid pharmaceutical composition comprising an Annexin A1 (AnxA1) N-terminal-peptide and at least one pharmaceutically acceptable excipient for use in the treatment of an ischemic condition and/or an inflammatory condition, wherein the liquid pharmaceutical composition is administered to a human subject as a 1-120 minutes intravenous (i.v.) infusion, and wherein the AnxA1 N-terminal-peptide has a terminal elimination half-life (t_{1/2}) of at least 30 minutes after i.v. infusion.

### DESCRIPTION OF DRAWINGS

FIG. 1 shows mean PK profiles of Peptide 1 by dose level. Mean Peptide 1 concentrations (ng/mL) vs. time after a single 30-min Peptide 1 intravenous (i.v.) infusion corresponding to the different dose levels (25 μg/kg, 50 μg/kg, 100 μg/kg, 150 μg/kg, 200 μg/kg and 400 μg/kg) is shown.

### DEFINITIONS

The term “Water For Injection” (WFI) is used to refer to Sterile Water for Injection as per the USP standards.

The term “a” denotes at least one, i.e., one or more.

The term “dosing interval” as used herein refers to the time period between administration of two doses.

The terms “dose” and “dosage” are used interchangeable within this disclosure, A pharmaceutically acceptable excipient is understood as any excipient acceptable to include in a pharmaceutical composition. Pharmaceutically acceptable excipients include, but are not restricted to, bulking agents, tonicity agents, isotonicity modifiers, stabilisers, surfactants, buffers, water, carbohydrates and sugar alcohols.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering. The patient to be treated is preferably a mammal, in particular a human being. The patients to be treated can be of various ages.

The terms “approximately” and “about” as referred herein are synonymous. In some embodiments, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%, ±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration ±1%. In some embodiments, “approximately” and “about” refer to the listed amount, value, or duration ±0.5%. In some embodiments, “approximately” and “about” refer to the listed amount, value, or duration ±0.1%.

### DETAILED DESCRIPTION

It is an aspect of the present disclosure to provide a liquid pharmaceutical composition comprising an Annexin A1 (AnxA1) N-terminal-peptide and at least one pharmaceutically acceptable excipient, wherein the liquid pharmaceutical composition is to be administered to a human subject as a 1-120 minutes intravenous (i.v.) infusion, and wherein the AnxA1 N-terminal-peptide has a terminal elimination half-life (t_{1/2}) of at least 30 minutes after said i.v. infusion.

It is also an aspect of the present disclosure to provide a liquid pharmaceutical composition comprising an Annexin A1 (AnxA1) N-terminal-peptide and at least one pharmaceutically acceptable excipient for use in the treatment of an ischemic condition and/or an inflammatory condition, wherein the liquid pharmaceutical composition is administered to a human subject as a 1-120 minutes intravenous (i.v.) infusion, and wherein the AnxA1 N-terminal-peptide has a terminal elimination half-life (t_{1/2}) of at least 30 minutes after said i.v. infusion.

In some embodiments, the present disclosure relates to a method of treating an ischemic condition and/or an inflammatory condition, said method comprising administration of a liquid pharmaceutical composition comprising an Annexin A1 (AnxA1) N-terminal-peptide and at least one pharmaceutically acceptable excipient to a human subject as a 1-120 minutes intravenous (i.v.) infusion, and wherein the AnxA1 N-terminal-peptide has a terminal elimination half-life (t_{1/2}) of at least 30 minutes after said i.v. infusion.

In some embodiments, the present disclosure relates to the use of a liquid pharmaceutical composition comprising an Annexin A1 (AnxA1) N-terminal-peptide and at least one pharmaceutically acceptable excipient for the manufacture of a medicament for use in the treatment of an ischemic condition and/or an inflammatory condition, wherein the liquid pharmaceutical composition is administered to a human subject as a 1-120 minutes intravenous (i.v.) infusion, and wherein the AnxA1 N-terminal-peptide has a terminal elimination half-life (t_{1/2}) of at least 30 minutes after i.v. infusion.

An intravenous infusion is the direct injection of a fluid medication into a vein through an intravenous line, needle, cannula or catheter. Administration of drugs via IV infusion is usually necessary when longer or continuous systemic exposures are required to elicit a therapeutic effect. Unlike injection, infusion often uses a pump or the natural force of gravity to deliver fluids into the body.

In some embodiments reference to a 1-120 minutes intravenous (i.v.) infusion encompass reference to a continuous 1-120 minutes intravenous (i.v.) infusion to a human subject.

In some embodiments, the liquid pharmaceutical composition is to be administered to said human subject as a 1-120 minutes i.v. infusion, such as about a 1-10 minutes i.v. infusion, such as about a 10-20 minutes i.v. infusion, such as about a 20-30 minutes i.v. infusion, such as about a 30-40 minutes i.v. infusion, such as about a 40-50 minutes i.v. infusion, such as about a 50-60 minutes i.v. infusion such as about a 60-70 minutes i.v. infusion, such as about a 70-80 minutes i.v. infusion, such as about a 80-90 minutes i.v. infusion such as about a 90-100 minutes i.v. infusion, such as about a 100-110 minutes i.v. infusion, such as about a 110-120 minutes i.v. infusion.

In some embodiments, the liquid pharmaceutical composition is to be administered to said human subject as a 10-90 minutes i.v. infusion, such as a 10-60 minutes i.v. infusion, such as a 20-40 minutes i.v. infusion, such as a 30 minutes i.v. infusion.

In some embodiments, the liquid pharmaceutical composition is to be administered to said human subject as an about 30 minutes i.v. infusion.

In some embodiment the AnxA1 N-terminal-peptide has a terminal elimination half-life (t_{1/2}) of at least 30 minutes after said i.v. infusion.

In some embodiments, the AnxA1 N-terminal-peptide has a t_{1/2} of at least 35 minutes, such as at least 40 minutes, such as at least 45 minutes, such as at least 50 minutes, such as at least 55 minutes, such as at least 60 minutes after said i.v. infusion.

In some embodiments, the AnxA1 N-terminal-peptide has a t_{1/2} of at least 35 minutes, such as at least 45 minutes, such as at least 60 minutes, such as at least 75 minutes, such as at least 90 minutes, such as at least 120 minutes, such as at least 180 minutes after said i.v. infusion.

In some embodiments, the AnxA1 N-terminal-peptide has a t_{1/2} of about 30-180 minutes after said i.v. infusion, such as about 30-40 minutes, such as about 40-50 minutes, such as about 50-60 minutes, such as about 60-75 minutes, such as about 75-90 minutes, such as about 90-105 minutes, such as about 105-120 minutes, such as about 120-135 minutes, such as about 135-150 minutes, such as about 150-165 minutes, such as about 165-180 minutes after said i.v. infusion.

In some embodiments, the i.v. infusion corresponds to administration of a dosage of about 15 μg/kg, about 25 μg/kg, about 50 μg/kg, about 100 μg/kg, about 150 μg/kg, about 200 μg/kg or about 400 μg/kg of the AnxA1 N-terminal-peptide of the present disclosure.

In some embodiments, the i.v. infusion corresponds to administration of a dosage of about 15 μg/kg of the AnxA1 N-terminal-peptide of the present disclosure. In some embodiments, the i.v. infusion corresponds to administration of a dosage of about 25 μg/kg of the AnxA1 N-terminal-peptide of the present disclosure. In some embodiments, the i.v. infusion corresponds to administration of a dosage of about 50 μg/kg of the AnxA1 N-terminal-peptide of the present disclosure. In some embodiments, the i.v. infusion corresponds to administration of a dosage of about 100 μg/kg of the AnxA1 N-terminal-peptide of the present disclosure. In some embodiments, the i.v. infusion corresponds to administration of a dosage of about 150 μg/kg of the AnxA1 N-terminal-peptide of the present disclosure. In some embodiments, the i.v. infusion corresponds to administration of a dosage of about 200 μg/kg of the AnxA1 N-terminal-peptide of the present disclosure. In some embodiments, the i.v. infusion corresponds to administration of a dosage of about 400 μg/kg of the AnxA1 N-terminal-peptide of the present disclosure.

In some embodiments, the AnxA1 N-terminal-peptide of the present disclosure is selected from the group consisting of:
a. AMVSEFLKQAWFIENEEQEYVQTVKSSKGGPGSAVSPYPTFNPSSDVAA (SEQ ID NO: 2; Annexin A1 2-50),
b. AMVSEFLKQAWFIENEEQEYVQTVKSSKGGPGSAVSPYPTFNPSSDV (SEQ ID NO: 3; Annexin A1 2-48),
c. AMVSEFLKQAWFIENEEQEYVQTVKSSKGGPGSAVSPYPTFNPSS (SEQ ID NO: 4; Annexin A1 2-46),
d. AMVSEFLKQAWFIENEEQEYVQTLKSSKGGPGSAVSPYPTFNPSSDVAA (SEQ ID NO: 5; Annexin A1 2-50 V24L),
e. AMVSEFLKQAWFIENEEQEYVQTLKSSKGGPGSAVSPYPTFNPSSDV (SEQ ID NO: 6; Annexin A1 2-48 V24L),
f. AMVSEFLKQAWFIENEEQEYVQTLKSSKGGPGSAVSPYPTFNPSS (SEQ ID NO: 7; Annexin A1 2-46 V24L), and
g. a functional variant of any one of SEQ ID NOs: 2-7 comprising 1 to 6 individual amino acid substitutions.

In some embodiments, the functional variant of any one of SEQ ID NOs: 2-7 comprises 1-3 individual amino acid substitutions, such as 1 individual amino acid substitution, 2 individual amino acid substitutions or 3 individual amino acid substitutions.

In some embodiments said individual amino acid substitution is a conservative amino acid substitution. In some embodiments, the functional variant of any one of SEQ ID NOs: 2-7 comprises 1-3 conservative amino acid substitutions, such as 1 conservative amino acid substitution, 2 conservative amino acid substitutions or 3 conservative amino acid substitutions.

In some embodiments, the functional variant of any one of SEQ ID NOs: 2-7 is a ligand and/or agonist of one or more of Formyl Peptide Receptor 1 (FPR1), Formyl Peptide Receptor 2 (FPR2) and Formyl Peptide Receptor 3 (FPR3).

In some embodiments, the functional variant of any one of SEQ ID NOs: 2-7 activates and/or stimulates one or more of Formyl Peptide Receptor 1 (FPR1), Formyl Peptide Receptor 2 (FPR2) and Formyl Peptide Receptor 3 (FPR3).

In some embodiments, the AnxA1 N-terminal-peptide or the functional variant thereof has a C-terminal amidation.

In some embodiments, the AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), optionally with a C-terminal amidation.

In some embodiments, the AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), with a C-terminal amidation.

In some embodiments, the liquid pharmaceutical composition comprises about 0.5-10 mg/ml of said AnxA1 N-terminal-peptide, such as about 1-10 mg/mL, such as about 2-9 mg/mL, such as about 3-8 mg/mL, such as about 4-6 mg/mL.

In some embodiments, the liquid pharmaceutical composition comprises about 5 mg/mL of the AnxA1 N-terminal-peptide.

In some embodiments, the liquid pharmaceutical composition has pH>6.0.

In some embodiments, the liquid pharmaceutical composition has pH≥7.4, such as pH≥7.5, such as pH≥7.6, such as pH≥7.7, such as pH≥7.8, such as pH≥7.9, such as pH≥8.0. In some embodiments, the liquid pharmaceutical composition has pH>8.0.

In some embodiments, the liquid pharmaceutical composition has a pH of about 8.2-8.4. In some embodiments, the liquid pharmaceutical composition has a pH of about 8.3.

In some embodiments, the liquid pharmaceutical composition comprises:
i) a sugar alcohol, and/or
ii) a carbohydrate.

Sugar alcohols and carbohydrates share the same feature in their backbones, i.e., —CHOH—CHOH—. The sugar alcohols include such compounds as sorbitol, mannitol, glycerol, and polyethylene glycols (PEGs). These compounds are straight-chain molecules. The carbohydrates, such as sucrose, mannose, ribose, trehalose, maltose, glycerol, inositol, glucose and lactose, on the other hand, are cyclic molecules that may contain a keto or aldehyde group. These two classes of compounds are effective in stabilizing protein against denaturation caused by elevated temperature and by freeze-thaw or freeze-drying processes.

In some embodiments, the liquid pharmaceutical composition comprises a carbohydrate, a sugar alcohol, or a combination of a carbohydrate and a sugar alcohol.

In some embodiments, the liquid pharmaceutical composition comprises at least one carbohydrate, at least one sugar alcohol, or a combination of at least one carbohydrate and at least one sugar alcohol.

In some embodiments, the one or more sugar alcohols serve as a bulking agent, tonicity agent, isotonicity modifier or stabiliser. In some embodiments, the one or more carbohydrates serve as a bulking agent, tonicity agent, isotonicity modifier or stabiliser.

In some embodiments, the liquid pharmaceutical composition comprises:
i) a sugar alcohol selected from the group consisting of mannitol, sorbitol, inositol, glycerol, xylitol, propylene glycol, polyethylene glycols (PEGs) and polypropylene/ethylene glycol copolymer, and/or
ii) a carbohydrate selected from the group consisting of sucrose, trehalose, mannose, ribose, maltose, glucose, lactose, galactose and arabinose.

In some embodiments, the liquid pharmaceutical composition comprises mannitol, such as approximately 4% (w/v) mannitol or approximately 5% (w/v) mannitol. In some embodiments, the liquid pharmaceutical composition comprises 4 to 6% (w/v) mannitol, such as 4 to 6% (w/v) mannitol, such as 4 to 4.5, such as 4.5 to 5, such as 5 to 5.5, such as 5.5 to 6% (w/v) mannitol. In some embodiments, the liquid pharmaceutical composition comprises 3 to 5% (w/v) mannitol, such as 3 to 5% (w/v) mannitol, such as 3 to 3.5, such as 3.5 to 4, such as 4 to 4.5, such as 4.5 to 4% (w/v) mannitol. In some embodiments, the liquid pharmaceutical composition comprises 0-10% (w/v) mannitol, such as 0.1-1, such as 1-2, such as 2-3, such as 3-4, such as 4-5, such as 5-6, such as 6-7, such as 7-8, such as 8-9, such as 9-10% (w/v) mannitol.

In some embodiments, the liquid pharmaceutical composition comprises sucrose, such as approximately 1% (w/v) sucrose. In some embodiments, the liquid pharmaceutical composition comprises 1-5% (w/v) sucrose, such as 1-2, such as 2-3, such as 3-4, such as 4-5% (w/v) sucrose.

In some embodiments, the liquid pharmaceutical composition comprises:
i) 1-10 mg/ml of an AnxA1 N-terminal-peptide of the present disclosure,
ii) 3-5% (w/w) of a sugar alcohol, and
iii) 0.5-1.5% (w/w) of a carbohydrate.

In some embodiments, the liquid pharmaceutical composition comprises:
i) 1-10 mg/ml of an AnxA1 N-terminal-peptide of the present disclosure,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer, and
iii) 0.5-1.5% (w/w) of a carbohydrate selected from the group consisting of sucrose, trehalose, mannose, ribose, maltose, glucose, lactose, galactose and arabinose.

In some embodiments, the liquid pharmaceutical composition comprises:
i) 1-10 mg/mL of an AnxA1 N-terminal-peptide of the present disclosure,
ii) 3-5% (w/w) of a sugar alcohol,
iii) 0.5-1.5% (w/w) of a carbohydrate,
iv) 0.005-0.015% (w/w) of a surfactant, and
v) 5-15 mM buffer.

In some embodiments, the liquid pharmaceutical composition comprises:
i) 1-10 mg/ml of an AnxA1 N-terminal-peptide of the present disclosure,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer,
iii) 0.5-1.5% (w/w) of a carbohydrate selected from the group consisting of sucrose, trehalose, mannose, ribose, maltose, glucose, lactose, galactose and arabinose,
iv) 0.005-0.015% (w/w) of a surfactant selected from the group consisting of Polysorbate-80 and Polysorbate-20, and
v) 5-15 mM buffer selected from the group consisting of Acetate buffer, TRIS, Amine-containing buffers, Amino-acid based buffers such as lysine, hydroxy-lysine histidine and glycylglycine, non-phosphate buffers, bicarbonate buffers, HEPES and borate buffers.

In some embodiments, the liquid pharmaceutical composition comprises sterile water for injection.

In some embodiments, the liquid pharmaceutical composition comprises glycyl-glycine and Polysorbate-80 or Polysorbate-20. In some embodiments, the liquid pharmaceutical composition comprises 1 to 20 mM glycyl-glycine and 0.001 to 0.1% Polysorbate-80 or Polysorbate-20. In some embodiments, the liquid pharmaceutical composition comprises 10 mM glycyl-glycine and 0.01% Polysorbate-80 or Polysorbate-20.

In some embodiments, the liquid pharmaceutical composition comprises:
i) about 5 mg/ml of a peptide having the sequence AMVSEFLKQAWFIENEEQEYVQTLKSSKGGPGSAVSPYPTFNPSSDV (SEQ ID NO: 6; Annexin A1 2-48 V24L), optionally with a C-terminal amidation,
ii) about 4% (w/w) mannitol,
iii) about 1% (w/w) sucrose,
iv) about 0.01% (w/w) Polysorbate-80,
v) 10 mM glycyl-glycine, and
vi) sterile water for injection.

In some embodiments, the liquid pharmaceutical composition is administered to a human subject as a once daily i.v. infusion.

In some embodiments, the liquid pharmaceutical composition is administered as multiple daily i.v. infusions, such as 2, 3, 4 or 5 daily i.v. infusions.

In some embodiments, the liquid pharmaceutical composition is to be administered as three daily i.v. infusions.

In some embodiments, the liquid pharmaceutical composition is to be administered as three daily i.v. infusions with a dosing interval of 4 to 12 hours, such as a dosing interval of 5 to 11 hours, such as a dosing interval of 6 to 10 hours, such as a dosing interval of 7 to 9 hours.

In some embodiments, the liquid pharmaceutical composition is to be administered as three daily i.v. infusions with a dosing interval of 6 to 10 hours, such as a dosing interval of 6 hours and 30 minutes to 9 hours and 30 minutes, such as 7 to 9 hours, such as a dosing interval of 7 hours and 15 minutes to 8 hours and 45 minutes, such as a dosing interval of 7 hours and 30 minutes to 8 hours and 30 minutes, such as a dosing interval of 7 hours and 45 minutes to 8 hours and 15 minutes.

In some embodiments, the liquid pharmaceutical composition is to be administered as three daily i.v. infusions with a dosing interval of about 8 hours.

In some embodiments, the once daily i.v. infusion or each of the multiple daily i.v. infusions correspond to administration of a dosage of about 15 μg/kg, about 25 μg/kg, about 50 μg/kg, about 100 μg/kg, about 150 μg/kg, about 200 μg/kg, about 250 μg/kg, about 300 μg/kg, about 350 μg/kg or about 400 μg/kg of the AnxA1 N-terminal-peptide.

In some embodiments, the once daily i.v. infusion or each of the multiple daily i.v. infusions correspond to administration of a dosage of about 15-400 μg/kg, about 25-400 μg/kg, about 50-400 μg/kg, about 100-400 μg/kg, about 150-400 μg/kg, or about 200-400 μg/kg of the AnxA1 N-terminal-peptide.

In some embodiments, the once daily i.v. infusion or each of the multiple daily i.v. infusions correspond to administration of a dosage of about 15-400 μg/kg, about 25-400 μg/kg, about 25-200 μg/kg, about 25-150 μg/kg, about 25-100 μg/kg, or about 25-50 μg/kg of the AnxA1 N-terminal-peptide.

In some embodiments, the once daily i.v. infusion or each of the multiple daily i.v. infusions correspond to administration of a dosage of about 25-200 μg/kg of the AnxA1 N-terminal-peptide.

In some embodiments, the once daily i.v. infusion or each of the multiple daily i.v. infusions correspond to administration of a dosage of about 25-200 μg/kg, about 50-200 μg/kg, about 75-200 μg/kg, about 100-200 μg/kg, about 125-200 μg/kg, or about 150-200 μg/kg.

In some embodiments, the once daily i.v. infusion or each of the multiple daily i.v. infusions correspond to administration of a dosage of about 25-200 μg/kg, about 25-175 μg/kg, about 25-150 μg/kg, about 25-125 μg/kg, about 25-100 μg/kg, or about 25-75 μg/kg.

In some embodiments, the once daily i.v. infusion or each of the multiple daily i.v. infusions correspond to administration of a dosage of about 200-400 μg/kg, about 250-400 μg/kg, about 300-400 μg/kg, or about 350-400 μg/kg.

In some embodiments, the once daily i.v. infusion or each of the multiple daily i.v. infusions correspond to administration of a dosage of about 150-350 μg/kg, about 160-340 μg/kg, about 170-330 μg/kg, about 180-320 μg/kg, about 190-310 μg/kg, about 200-300 μg/kg, about 210-290 μg/kg, about 220-280 μg/kg, about 230-270 μg/kg, or about 240-260 μg/kg.

In some embodiments, the once daily i.v. infusion or multiple daily i.v. infusions correspond to administration of a total dosage of about 200-800 μg/kg, about 200-750 μg/kg, about 200-700 μg/kg, about 200-650 μg/kg, about 200-600 μg/kg, about 200-550 μg/kg, about 200-500 μg/kg, about 200-450 μg/kg, or about 200-400 μg/kg.

In some embodiments, the once daily i.v. infusion or multiple daily i.v. infusions correspond to administration of a total dosage of about 200-800 μg/kg, about 250-800 μg/kg, about 300-800 μg/kg, about 350-800 μg/kg, about 400-800 μg/kg, about 450-800 μg/kg, about 500-800 μg/kg, about 550-800 μg/kg, or about 600-800 μg/kg.

In some embodiments, the liquid pharmaceutical composition is administered as 3 times daily i.v. infusions.

In some embodiments, the liquid pharmaceutical composition is administered 3 times daily as a 20-40 minute i.v. infusion, such as a 30 minute i.v. infusion.

In some embodiments, each i.v. infusion corresponds to administration of a dosage of about 25-200 μg/kg, about 25-150 μg/kg, about 25-100 μg/kg, or about 25-50 μg/kg of the AnxA1 N-terminal-peptide.

In some embodiments, each i.v. infusion corresponds to administration of a dosage of about 50 μg/kg of the AnxA1 N-terminal-peptide.

In some embodiments, each i.v. infusion corresponds to administration of a dosage of 50-250 μg/kg, such as 50-200 μg/kg, such as 50-150 μg/kg, such as 50-140 μg/kg, such as 60-120 μg/kg.

In some embodiments, the three daily i.v. infusions are administered at time 0 hours, 8 hours and 16 hours.

In some embodiments, the mean AUC_{0-24 hrs} of the liquid pharmaceutical composition is selected from the group consisting of:
i) within about 80.00% to about 125.00% of an AUC_{0-24 hrs} of 211 (CV 2.6%) h·ng/ml, after administration of a single dose of 25 μg/kg,
ii) within about 80.00% to about 125.00% of an AUC_{0-24 hrs} of 464 (CV 18.8%) h·ng/mL, after administration of a single dose of 50 μg/kg,
iii) within about 80.00% to about 125.00% of an AUC_{0-24 hrs} of 1108 (CV 13.4%) h·ng/ml, after administration of a single dose of 100 μg/kg,
iv) within about 80.00% to about 125.00% of an AUC_{0-24 hrs} of 1521 (CV 22.6%) h·ng/ml, after administration of a single dose of 150 μg/kg,
v) within about 80.00% to about 125.00% of an AUC_{0-24 hrs} of 2278 (CV 3.1%) h·ng/ml, after administration of a single dose of 200 μg/kg, and
vi) within about 80.00% to about 125.00% of an AUC_{0-24 hrs} of 4872 (CV 11.4%) h·ng/ml, after administration of a single dose of 400 μg/kg.

In some embodiments, the mean AUC_{0-24 hrs} of the liquid pharmaceutical composition is within about 80.00% to about 125.00% of an AUC_{0-24 hrs} of 211 (CV 2.6%) h·ng/ml, after administration of a single dose of 25 μg/kg.

In some embodiments, the mean AUC_{0-24 hrs} of the liquid pharmaceutical composition is within about 80.00% to about 125.00% of an AUC_{0-24 hrs} of 464 (CV 18.8%) h·ng/mL, after administration of a single dose of 50 μg/kg.

In some embodiments, the mean AUC_{0-24 hrs} of the liquid pharmaceutical composition is within about 80.00% to about 125.00% of an AUC_{0-24 hrs} of 1108 (CV 13.4%) h·ng/ml, after administration of a single dose of 100 μg/kg.

In some embodiments, the mean AUC_{0-24 hrs} of the liquid pharmaceutical composition is within about 80.00% to about 125.00% of an AUC_{0-24 hrs} of 1521 (CV 22.6%) h·ng/ml, after administration of a single dose of 150 μg/kg.

In some embodiments, the mean AUC_{0-24 hrs} of the liquid pharmaceutical composition is within about 80.00% to about 125.00% of an AUC_{0-24 hrs} of 2278 (CV 3.1%) h·ng/ml, after administration of a single dose of 200 μg/kg.

In some embodiments, the mean AUC_{0-24 hrs} of the liquid pharmaceutical composition is within about 80.00% to about 125.00% of an AUC_{0-24 hrs} of 4872 (CV 11.4%) h·ng/ml, after administration of a single dose of 400 μg/kg.

In some embodiments, the geometric mean AUC_{0-24 hrs} of the liquid pharmaceutical composition is selected from the group consisting of:
i) within about 80.00% to about 125.00% of an AUC_{0-24 hrs} of 211 (CV 2.6%) h·ng/ml, after administration of a single dose of 25 μg/kg,
ii) within about 80.00% to about 125.00% of an AUC_{0-24 hrs} of 457 (CV 18.8%) h·ng/ml, after administration of a single dose of 50 μg/kg,
iii) within about 80.00% to about 125.00% of an AUC_{0-24 hrs} of 1099 (CV 13.4%) h·ng/ml, after administration of a single dose of 100 μg/kg,
iv) within about 80.00% to about 125.00% of an AUC_{0-24 hrs} of 1489 (CV 22.6%) h·ng/ml, after administration of a single dose of 150 μg/kg,
v) within about 80.00% to about 125.00% of an AUC_{0-24 hrs} of 2278 (CV 3.1%) h·ng/ml, after administration of a single dose of 200 μg/kg, and
vi) within about 80.00% to about 125.00% of an AUC_{0-24 hrs} of 4844 (CV 11.4%) h·ng/ml, after administration of a single dose of 400 μg/kg.

In some embodiments, the geometric mean AUC_{0-24 hrs} of the liquid pharmaceutical composition is within about 80.00% to about 125.00% of an AUC_{0-24 hrs} of 211 (CV 2.6%) h·ng/ml, after administration of a single dose of 25 μg/kg.

In some embodiments, the geometric mean AUC_{0-24 hrs} of the liquid pharmaceutical composition is within about 80.00% to about 125.00% of an AUC_{0-24 hrs} of 457 (CV 18.8%) h·ng/ml, after administration of a single dose of 50 μg/kg.

In some embodiments, the geometric mean AUC_{0-24 hrs} of the liquid pharmaceutical composition is within about 80.00% to about 125.00% of an AUC_{0-24 hrs} of 1099 (CV 13.4%) h·ng/ml, after administration of a single dose of 100 μg/kg.

In some embodiments, the geometric mean AUC_{0-24 hrs} of the liquid pharmaceutical composition is within about 80.00% to about 125.00% of an AUC_{0-24 hrs} of 1489 (CV 22.6%) h·ng/ml, after administration of a single dose of 150 μg/kg.

In some embodiments, the geometric mean AUC_{0-24 hrs} of the liquid pharmaceutical composition is within about 80.00% to about 125.00% of an AUC_{0-24 hrs} of 2278 (CV 3.1%) h·ng/ml, after administration of a single dose of 200 μg/kg.

In some embodiments, the geometric mean AUC_{0-24 hrs} of the liquid pharmaceutical composition is within about 80.00% to about 125.00% of an AUC_{0-24 hrs} of 4844 (CV 11.4%) h·ng/mL, after administration of a single dose of 400 μg/kg.

In some embodiments, the mean AUC₀₋ₜ of the liquid pharmaceutical composition is selected from the group consisting of:
i) within about 80.00% to about 125.00% of an AUC₀₋ₜ of 133 (CV 19.4%) h·ng/ml, after administration of a single dose of 25 μg/kg,
ii) within about 80.00% to about 125.00% of an AUC₀₋ₜ of 378 (CV 22.4%) h·ng/ml, after administration of a single dose of 50 μg/kg,
iii) within about 80.00% to about 125.00% of an AUC₀₋ₜ of 1001 (CV 15.2%) h·ng/ml, after administration of a single dose of 100 μg/kg,
iv) within about 80.00% to about 125.00% of an AUC₀₋ₜ of 1399 (CV 22.0%) h·ng/ml, after administration of a single dose of 150 μg/kg,
v) within about 80.00% to about 125.00% of an AUC₀₋ₜ of 2119 (CV 5.2%) h·ng/ml, after administration of a single dose of 200 μg/kg, and
vi) within about 80.00% to about 125.00% of an AUC₀₋ₜ of 4719 (CV 11.8%) h·ng/mL, after administration of a single dose of 400 μg/kg.

In some embodiments, the mean AUC₀₋ₜ of the liquid pharmaceutical composition is within about 80.00% to about 125.00% of an AUC₀₋ₜ of 133 (CV 19.4%) h·ng/ml, after administration of a single dose of 25 μg/kg.

In some embodiments, the mean AUC₀₋ₜ of the liquid pharmaceutical composition is within about 80.00% to about 125.00% of an AUC₀₋ₜ of 378 (CV 22.4%) h·ng/ml, after administration of a single dose of 50 μg/kg.

In some embodiments, the mean AUC₀₋ₜ of the liquid pharmaceutical composition is within about 80.00% to about 125.00% of an AUC₀₋ₜ of 1001 (CV 15.2%) h·ng/mL, after administration of a single dose of 100 μg/kg.

In some embodiments, the mean AUC₀₋ₜ of the liquid pharmaceutical composition is within about 80.00% to about 125.00% of an AUC₀₋ₜ of 1399 (CV 22.0%) h·ng/ml, after administration of a single dose of 150 μg/kg.

In some embodiments, the mean AUC₀₋ₜ of the liquid pharmaceutical composition is within about 80.00% to about 125.00% of an AUC₀₋ₜ of 2119 (CV 5.2%) h·ng/ml, after administration of a single dose of 200 μg/kg.

In some embodiments, the mean AUC₀₋ₜ of the liquid pharmaceutical composition is within about 80.00% to about 125.00% of an AUC₀₋ₜ of 4719 (CV 11.8%) h·ng/ml, after administration of a single dose of 400 μg/kg.

In some embodiments, the geometric mean AUC₀₋ₜ of the liquid pharmaceutical composition is selected from the group consisting of:
i) within about 80.00% to about 125.00% of an AUC₀₋ₜ of 130 (CV 19.4%) h·ng/ml, after administration of a single dose of 25 μg/kg,
ii) within about 80.00% to about 125.00% of an AUC₀₋ₜ of 369 (CV 22.4%) h·ng/mL, after administration of a single dose of 50 μg/kg,
iii) within about 80.00% to about 125.00% of an AUC₀₋ₜ of 991 (CV 15.2%) h·ng/ml, after administration of a single dose of 100 μg/kg,
iv) within about 80.00% to about 125.00% of an AUC₀₋ₜ of 1371 (CV 22.0%) h·ng/ml, after administration of a single dose of 150 μg/kg,
v) within about 80.00% to about 125.00% of an AUC₀₋ₜ of 2116 (CV 5.2%) h·ng/ml, after administration of a single dose of 200 μg/kg, and
vi) within about 80.00% to about 125.00% of an AUC₀₋ₜ of 4689 (CV 11.8%) h·ng/mL, after administration of a single dose of 400 μg/kg.

In some embodiments, the geometric mean AUC₀₋ₜ of the liquid pharmaceutical composition is within about 80.00% to about 125.00% of an AUC₀₋ₜ of 130 (CV 19.4%) h·ng/ml, after administration of a single dose of 25 μg/kg.

In some embodiments, the geometric mean AUC₀₋ₜ of the liquid pharmaceutical composition is within about 80.00% to about 125.00% of an AUC₀₋ₜ of 369 (CV 22.4%) h·ng/ml, after administration of a single dose of 50 μg/kg.

In some embodiments, the geometric mean AUC₀₋ₜ of the liquid pharmaceutical composition is within about 80.00% to about 125.00% of an AUC₀₋ₜ of 991 (CV 15.2%) h·ng/ml, after administration of a single dose of 100 μg/kg.

In some embodiments, the geometric mean AUC₀₋ₜ of the liquid pharmaceutical composition is within about 80.00% to about 125.00% of an AUC₀₋ₜ of 1371 (CV 22.0%) h·ng/ml, after administration of a single dose of 150 μg/kg.

In some embodiments, the geometric mean AUC₀₋ₜ of the liquid pharmaceutical composition is within about 80.00% to about 125.00% of an AUC₀₋ₜ of 2116 (CV 5.2%) h·ng/ml, after administration of a single dose of 200 μg/kg.

In some embodiments, the geometric mean AUC₀₋ₜ of the liquid pharmaceutical composition is within about 80.00% to about 125.00% of an AUC₀₋ₜ of 4689 (CV 11.8%) h·ng/ml, after administration of a single dose of 400 μg/kg.

In some embodiments, the mean AUC_{0-infinity} of the liquid pharmaceutical composition is selected from the group consisting of:
i) within about 80.00% to about 125.00% of an AUC_{0-infinity} of 211 (CV 19.4%) h·ng/mL, after administration of a single dose of 25 μg/kg,
ii) within about 80.00% to about 125.00% of an AUC_{0-infinity} of 464 (CV 22.4%) h·ng/mL, after administration of a single dose of 50 μg/kg,
iii) within about 80.00% to about 125.00% of an AUC_{0-infinity} of 1108 (CV 15.2%) h·ng/ml, after administration of a single dose of 100 μg/kg,
iv) within about 80.00% to about 125.00% of an AUC_{0-infinity} of 1521 (CV 22.0%) h·ng/ml, after administration of a single dose of 150 μg/kg,
v) within about 80.00% to about 125.00% of an AUC_{0-infinity} of 2278 (CV 5.2%) h·ng/mL, after administration of a single dose of 200 μg/kg, and
vi) within about 80.00% to about 125.00% of an AUC_{0-infinity} of 4873 (CV 11.8%) h·ng/ml, after administration of a single dose of 400 μg/kg.

In some embodiments, the mean AUC_{0-infinity} of the liquid pharmaceutical composition is within about 80.00% to about 125.00% of an AUC_{0-infinity} of 211 (CV 19.4%) h·ng/ml, after administration of a single dose of 25 μg/kg.

In some embodiments, the mean AUC_{0-infinity} of the liquid pharmaceutical composition is within about 80.00% to about 125.00% of an AUC_{0-infinity} of 464 (CV 22.4%) h·ng/mL, after administration of a single dose of 50 μg/kg.

In some embodiments, the mean AUC_{0-infinity} of the liquid pharmaceutical composition is within about 80.00% to about 125.00% of an AUC_{0-infinity} of 1108 (CV 15.2%) h·ng/ml, after administration of a single dose of 100 μg/kg.

In some embodiments, the mean AUC_{0-infinity} of the liquid pharmaceutical composition is within about 80.00% to about 125.00% of an AUC_{0-infinity} of 1521 (CV 22.0%) h·ng/ml, after administration of a single dose of 150 μg/kg.

In some embodiments, the mean AUC_{0-infinity} of the liquid pharmaceutical composition is within about 80.00% to about 125.00% of an AUC_{0-infinity} of 2278 (CV 5.2%) h·ng/ml, after administration of a single dose of 200 μg/kg.

In some embodiments, the mean AUC_{0-infinity} of the liquid pharmaceutical composition is within about 80.00% to about 125.00% of an AUC_{0-infinity} of 4873 (CV 11.8%) h·ng/ml, after administration of a single dose of 400 μg/kg.

In some embodiments, the geometric mean AUC_{0-infinity} of the liquid pharmaceutical composition is selected from the group consisting of:
i) within about 80.00% to about 125.00% of an AUC_{0-infinity} of 211 (CV 19.4%) h·ng/mL, after administration of a single dose of 25 μg/kg,
ii) within about 80.00% to about 125.00% of an AUC_{0-infinity} of 456 (CV 22.4%) h·ng/ml, after administration of a single dose of 50 μg/kg,
iii) within about 80.00% to about 125.00% of an AUC_{0-infinity} of 1099 (CV 15.2%) h·ng/ml, after administration of a single dose of 100 μg/kg,
iv) within about 80.00% to about 125.00% of an AUC_{0-infinity} of 1490 (CV 22.0%) h·ng/ml, after administration of a single dose of 150 μg/kg,
v) within about 80.00% to about 125.00% of an AUC_{0-infinity} of 2277 (CV 5.2%) h·ng/ml, after administration of a single dose of 200 μg/kg, and
vi) within about 80.00% to about 125.00% of an AUC_{0-infinity} of 4845 (CV 11.8%) h·ng/ml, after administration of a single dose of 400 μg/kg.

In some embodiments, the geometric mean AUC_{0-infinity} of the liquid pharmaceutical composition is within about 80.00% to about 125.00% of an AUC_{0-infinity} of 211 (CV 19.4%) h·ng/ml, after administration of a single dose of 25 μg/kg.

In some embodiments, the geometric mean AUC_{0-infinity} of the liquid pharmaceutical composition is within about 80.00% to about 125.00% of an AUC_{0-infinity} of 456 (CV 22.4%) h·ng/ml, after administration of a single dose of 50 μg/kg.

In some embodiments, the geometric mean AUC_{0-infinity} of the liquid pharmaceutical composition is within about 80.00% to about 125.00% of an AUC_{0-infinity} of 1099 (CV 15.2%) h·ng/ml, after administration of a single dose of 100 μg/kg.

In some embodiments, the geometric mean AUC_{0-infinity} of the liquid pharmaceutical composition is within about 80.00% to about 125.00% of an AUC_{0-infinity} of 1490 (CV 22.0%) h·ng/ml, after administration of a single dose of 150 μg/kg.

In some embodiments, the geometric mean AUC_{0-infinity} of the liquid pharmaceutical composition is within about 80.00% to about 125.00% of an AUC_{0-infinity} of 2277 (CV 5.2%) h·ng/ml, after administration of a single dose of 200 μg/kg.

In some embodiments, the geometric mean AUC_{0-infinity} of the liquid pharmaceutical composition is within about 80.00% to about 125.00% of an AUC_{0-infinity} of 4845 (CV 11.8%) h·ng/ml, after administration of a single dose of 400 μg/kg.

In some embodiments, the mean Cₘₐₓ of the liquid pharmaceutical composition is selected from the group consisting of:
i) within about 80.00% to about 125.00% of a Cₘₐₓ of 162 (CV 11.7%) ng/mL, after administration of a single dose of 25 μg/kg,
ii) within about 80.00% to about 125.00% of a Cₘₐₓ of 375 (CV 15.3%) ng/ml, after administration of a single dose of 50 μg/kg,
iii) within about 80.00% to about 125.00% of a Cₘₐₓ of 916 (CV 21.0%) ng/mL, after administration of a single dose of 100 μg/kg,
iv) within about 80.00% to about 125.00% of a Cₘₐₓ of 1280 (CV 23.3%) ng/mL, after administration of a single dose of 150 μg/kg,
v) within about 80.00% to about 125.00% of a Cₘₐₓ of 1700 (CV 4.4%) ng/ml, after administration of a single dose of 200 μg/kg, and
vi) within about 80.00% to about 125.00% of a Cₘₐₓ of 3420 (CV 10.7%) ng/ml, after administration of a single dose of 400 μg/kg.

In some embodiments, the mean Cₘₐₓ of the liquid pharmaceutical composition is within about 80.00% to about 125.00% of a Cₘₐₓ of 162 (CV 11.7%) ng/ml, after administration of a single dose of 25 μg/kg.

In some embodiments, the mean Cₘₐₓ of the liquid pharmaceutical composition is within about 80.00% to about 125.00% of a Cₘₐₓ of 375 (CV 15.3%) ng/ml, after administration of a single dose of 50 μg/kg.

In some embodiments, the mean Cₘₐₓ of the liquid pharmaceutical composition is within about 80.00% to about 125.00% of a Cₘₐₓ of 916 (CV 21.0%) ng/mL, after administration of a single dose of 100 g/kg.

In some embodiments, the mean Cₘₐₓ of the liquid pharmaceutical composition is within about 80.00% to about 125.00% of a Cₘₐₓ of 1280 (CV 23.3%) ng/ml, after administration of a single dose of 150 μg/kg.

In some embodiments, the present disclosure relates to the liquid pharmaceutical composition or the liquid pharmaceutical composition for use, wherein the mean Cₘₐₓ of the liquid pharmaceutical composition is within about 80.00% to about 125.00% of a Cₘₐₓ of 1700 (CV 4.4%) ng/ml, after administration of a single dose of 200 μg/kg.

In some embodiments, the mean Cₘₐₓ of the liquid pharmaceutical composition is within about 80.00% to about 125.00% of a Cₘₐₓ of 3420 (CV 10.7%) ng/ml, after administration of a single dose of 400 μg/kg.

In some embodiments, the geometric mean Cₘₐₓ of the liquid pharmaceutical composition is selected from the group consisting of:
i) within about 80.00% to about 125.00% of a Cₘₐₓ of 161 (CV 11.7%) ng/ml, after administration of a single dose of 25 μg/kg,
ii) within about 80.00% to about 125.00% of a Cₘₐₓ of 371 (CV 15.3%) ng/ml, after administration of a single dose of 50 μg/kg,
iii) within about 80.00% to about 125.00% of a Cₘₐₓ of 899 (CV 21.0%) ng/ml, after administration of a single dose of 100 μg/kg,
iv) within about 80.00% to about 125.00% of a Cₘₐₓ of 1250 (CV 23.3%) ng/ml, after administration of a single dose of 150 μg/kg,
v) within about 80.00% to about 125.00% of a Cₘₐₓ of 1700 (CV 4.4%) ng/ml, after administration of a single dose of 200 μg/kg, and
vi) within about 80.00% to about 125.00% of a Cₘₐₓ of 3400 (CV 10.7%) ng/ml, after administration of a single dose of 400 μg/kg.

In some embodiments, the geometric mean Cₘₐₓ of the liquid pharmaceutical composition is within about 80.00% to about 125.00% of a Cₘₐₓ of 161 (CV 11.7%) ng/mL, after administration of a single dose of 25 μg/kg.

In some embodiments, the geometric mean Cₘₐₓ of the liquid pharmaceutical composition is within about 80.00% to about 125.00% of a Cₘₐₓ of 371 (CV 15.3%) ng/ml, after administration of a single dose of 50 μg/kg.

In some embodiments, the geometric mean Cₘₐₓ of the liquid pharmaceutical composition is within about 80.00% to about 125.00% of a Cₘₐₓ of 899 (CV 21.0%) ng/mL, after administration of a single dose of 100 μg/kg.

In some embodiments, the geometric mean Cₘₐₓ of the liquid pharmaceutical composition is within about 80.00% to about 125.00% of a Cₘₐₓ of 1250 (CV 23.3%) ng/ml, after administration of a single dose of 150 μg/kg.

In some embodiments, the geometric mean Cₘₐₓ of the liquid pharmaceutical composition is within about 80.00% to about 125.00% of a Cₘₐₓ of 1700 (CV 4.4%) ng/ml, after administration of a single dose of 200 μg/kg.

In some embodiments, the geometric mean Cₘₐₓ of the liquid pharmaceutical composition is within about 80.00% to about 125.00% of a Cₘₐₓ of 3400 (CV 10.7%) ng/mL, after administration of a single dose of 400 μg/kg.

In some embodiments, reference to administration of a single dose is administration of a single dose as a 30-minute i.v. infusion to a human subject.

In some embodiments, the AUC_{0-24 hrs}, AUC₀₋ₜ, AUC_{0-24 infinity} or Cₘₐₓ is measured after administration of a single dose as 30-minute i.v. infusion to a human subject.

In some embodiments, the AUC_{0-24 hrs}, AUC₀₋ₜ, AUC_{0-24 infinity} or Cₘₐₓ is measured after administration of a single dose to a human subject, such as a healthy male Caucasian subject.

In some embodiments, the present disclosure relates to a method of achieving a t_{1/2}, AUC_{0-24 hrs}, AUC₀₋ₜ, AUC_{0-24 infinity} and/or Cₘₐₓ as disclosed herein by administering the liquid pharmaceutical composition to a human subject as a 1-120 minutes intravenous (i.v.) infusion.

In some embodiments, the mean AUC_{0-24 hrs} of the liquid pharmaceutical composition is selected from the group consisting of:
a. within about 80.00% to about 125.00% of an AUC_{0-24 hrs} of 1572 (CV 12.4%) h·ng/ml, after administration of three doses with a dosing interval of 8 hours, wherein each of said three doses corresponds to 50 μg/kg,
b. within about 80.00% to about 125.00% of an AUC_{0-24 hrs} of 3621 (CV 11.6%) h·ng/ml, after administration of three doses with a dosing interval of 8 hours, wherein each of said three doses corresponds to 100 μg/kg,
c. within about 80.00% to about 125.00% of an AUC_{0-24 hrs} of 5411 (CV 20.6%) h·ng/ml, after administration of three doses with a dosing interval of 8 hours, wherein each of said three doses corresponds to 140 μg/kg,
d. within about 80.00% to about 125.00% of an AUC_{0-24 hrs} of 8115 (CV 16.8%) h·ng/ml, after administration of three doses with a dosing interval of 8 hours, wherein each of said three doses corresponds to 200 μg/kg, and
e. within about 80.00% to about 125.00% of an AUC_{0-24 hrs} of 7551 (CV 13.7%) h·ng/ml, after administration of three doses with a dosing interval of 8 hours, wherein each of said three doses corresponds to 250 μg/kg.

In some embodiments, the mean AUC_{0-24 hrs} of the liquid pharmaceutical composition is within about 80.00% to about 125.00% of an AUC_{0-24 hrs} of 1572 (CV 12.4%) h·ng/ml, after administration of three doses with a dosing interval of 8 hours, wherein each of said three doses corresponds to 50 μg/kg.

In some embodiments, the mean AUC_{0-24 hrs} of the liquid pharmaceutical composition is within about 80.00% to about 125.00% of an AUC_{0-24 hrs} of 3621 (CV 11.6%) h·ng/ml, after administration of three doses with a dosing interval of 8 hours, wherein each of said three doses corresponds to 100 μg/kg.

In some embodiments, the mean AUC_{0-24 hrs} of the liquid pharmaceutical composition is within about 80.00% to about 125.00% of an AUC_{0-24 hrs} of of 5411 (CV 20.6%) h·ng/ml, after administration of three doses with a dosing interval of 8 hours, wherein each of said three doses corresponds to 140 μg/kg.

In some embodiments, the mean AUC_{0-24 hrs} of the liquid pharmaceutical composition is within about 80.00% to about 125.00% of an AUC_{0-24 hrs} of 8115 (CV 16.8%) h·ng/ml, after administration of three doses with a dosing interval of 8 hours, wherein each of said three doses corresponds to 200 μg/kg.

In some embodiments, the mean AUC_{0-24 hrs} of the liquid pharmaceutical composition is within about 80.00% to about 125.00% of an AUC_{0-24 hrs} of 7495 (CV 13.7%) h·ng/ml, after administration of three doses corresponding to 250 μg/kg with a dosing interval of 8 hours.

In some embodiments, the geometric mean AUC_{0-24 hrs} of the liquid pharmaceutical composition is selected from the group consisting of:
a. within about 80.00% to about 125.00% of an AUC_{0-24 hrs} of 1562 (CV 12.4%) h·ng/ml, after administration of three doses with a dosing interval of 8 hours, wherein each of said three doses corresponds to 50 μg/kg,
b. within about 80.00% to about 125.00% of an AUC_{0-24 hrs} of 3001 (CV 11.6%) h·ng/ml, after administration of three doses with a dosing interval of 8
c. within about 80.00% to about 125.00% of an AUC_{0-24 hrs} of 5310 (CV 20.6%) h·ng/ml, after administration of three doses with a dosing interval of 8 hours, wherein each of said three doses corresponds to 140 μg/kg,
d. within about 80.00% to about 125.00% of an AUC_{0-24 hrs} of 8119 (CV 16.8%) h·ng/ml, after administration of three doses with a dosing interval of 8 hours, wherein each of said three doses corresponds to 200 μg/kg, and
e. within about 80.00% to about 125.00% of an AUC_{0-24 hrs} of 7495 (CV 13.7%) h·ng/ml, after administration of three doses with a dosing interval of 8 hours, wherein each of said three doses corresponds to 250 μg/kg.

In some embodiments, the geometric mean AUC_{0-24 hrs} of the liquid pharmaceutical composition is within about 80.00% to about 125.00% of an AUC_{0-24 hrs} of 1562 (CV 12.4%) h·ng/ml, after administration of three doses with a dosing interval of 8 hours, wherein each of said three doses corresponds to 50 μg/kg.

In some embodiments, the geometric mean AUC_{0-24 hrs} of the liquid pharmaceutical composition is within about 80.00% to about 125.00% of an AUC_{0-24 hrs} of 3001 (CV 11.6%) h·ng/mL, after administration of three doses with a dosing interval of 8 hours, wherein each of said three doses corresponds to 100 μg/kg.

In some embodiments, the geometric mean AUC_{0-24 hrs} of the liquid pharmaceutical composition is within about 80.00% to about 125.00% of an AUC_{0-24 hrs} of 5310 (CV 20.6%) h·ng/ml, after administration of three doses with a dosing interval of 8 hours, wherein each of said three doses corresponds to 140 μg/kg.

In some embodiments, the geometric mean AUC_{0-24 hrs} of the liquid pharmaceutical composition is within about 80.00% to about 125.00% of an AUC_{0-24 hrs} of 8119 (CV 16.8%) h·ng/ml, after administration of three doses with a dosing interval of 8 hours, wherein each of said three doses corresponds to 200 μg/kg.

In some embodiments, the geometric mean AUC_{0-24 hrs} of the liquid pharmaceutical composition is within about 80.00% to about 125.00% of an AUC_{0-24 hrs} of 7495 (CV 13.7%) h·ng/ml, after administration of three doses corresponding to 250 μg/kg with a dosing interval of 8 hours.

In some embodiments, the mean AUC₀₋ₜ₁ of the liquid pharmaceutical composition is selected from the group consisting of:
a. within about 80.00% to about 125.00% of an AUC₀₋ₜ₁ of 422 (CV 15.8%) h·ng/ml, after administration of three doses with a dosing interval of 8 hours, wherein each of said three doses corresponds to 50 μg/kg,
b. within about 80.00% to about 125.00% of an AUC₀₋ₜ₁ of 993 (CV 16.2%) h·ng/ml, after administration of three doses with a dosing interval of 8 hours, wherein each of said three doses corresponds to 100 μg/kg,
c. within about 80.00% to about 125.00% of an AUC₀₋ₜ₁ of 1688 (CV 17.6%) h·ng/ml, after administration of three doses with a dosing interval of 8 hours, wherein each of said three doses corresponds to 140 μg/kg,
d. within about 80.00% to about 125.00% of an AUC₀₋ₜ₁ of 2520 (CV 17.5%) h·ng/mL, after administration of three doses with a dosing interval of 8 hours, wherein each of said three doses corresponds to 200 μg/kg, and
e. within about 80.00% to about 125.00% of an AUC₀₋ₜ₁ of 2540 (CV 19.8%) h·ng/ml, after administration of three doses with a dosing interval of 8 hours, wherein each of said three doses corresponds to 250 μg/kg.

In some embodiments, the mean AUC₀₋ₜ₁ of the liquid pharmaceutical composition is within about 80.00% to about 125.00% of an AUC₀₋ₜ₁ of 422 (CV 15.8%) h·ng/ml, after administration of three doses with a dosing interval of 8 hours, wherein each of said three doses corresponds to 50 μg/kg.

In some embodiments, the mean AUC₀₋ₜ₁ of the liquid pharmaceutical composition is within about 80.00% to about 125.00% of an AUC₀₋ₜ₁ of 993 (CV 16.2%) h·ng/ml, after administration of three doses with a dosing interval of 8 hours, wherein each of said three doses corresponds to 100 μg/kg.

In some embodiments, the mean AUC₀₋ₜ₁ of the liquid pharmaceutical composition is within about 80.00% to about 125.00% of an AUC₀₋ₜ₁ of 1688 (CV 17.6%) h·ng/ml, after administration of three doses with a dosing interval of 8 hours, wherein each of said three doses corresponds to 140 μg/kg.

In some embodiments, the mean AUC₀₋ₜ₁ of the liquid pharmaceutical composition is within about 80.00% to about 125.00% of an AUC₀₋ₜ₁ of 2520 (CV 17.5%) h·ng/ml, after administration of three doses with a dosing interval of 8 hours, wherein each of said three doses corresponds to 200 μg/kg.

In some embodiments, the mean AUC₀₋ₜ₁ of the liquid pharmaceutical composition is within about 80.00% to about 125.00% of an AUC₀₋ₜ₁ of 2540 (CV 19.8%) h·ng/ml, after administration of three doses corresponding to 250 μg/kg with a dosing interval of 8 hours.

In some embodiments, the geometric mean AUC₀₋ₜ₁ of the liquid pharmaceutical composition is selected from the group consisting of:
a. within about 80.00% to about 125.00% of an AUC₀₋ₜ₁ of 417 (CV 15.8%) h·ng/mL, after administration of three doses with a dosing interval of 8 hours, wherein each of said three doses corresponds to 50 μg/kg,
b. within about 80.00% to about 125.00% of an AUC₀₋ₜ₁ of 982 (CV 16.2%) h·ng/ml, after administration of three doses with a dosing interval of 8 hours, wherein each of said three doses corresponds to 100 μg/kg,
c. within about 80.00% to about 125.00% of an AUC₀₋ₜ₁ of 1664 (CV 17.6%) h·ng/ml, after administration of three doses with a dosing interval of 8 hours, wherein each of said three doses corresponds to 140 μg/kg,
d. within about 80.00% to about 125.00% of an AUC₀₋ₜ₁ of 2489 (CV 17.5%) h·ng/ml, after administration of three doses with a dosing interval of 8 hours, wherein each of said three doses corresponds to 200 μg/kg, and
e. within about 80.00% to about 125.00% of an AUC₀₋ₜ₁ of 2540 (CV 19.8%) h·ng/ml, after administration of three doses with a dosing interval of 8 hours, wherein each of said three doses corresponds to 250 μg/kg.

In some embodiments, the geometric mean AUC₀₋ₜ₁ of the liquid pharmaceutical composition is within about 80.00% to about 125.00% of an AUC₀₋ₜ₁ of 417 (CV 15.8%) h·ng/ml, after administration of three doses with a dosing interval of 8 hours, wherein each of said three doses corresponds to 50 μg/kg.

In some embodiments, the geometric mean AUC₀₋ₜ₁ of the liquid pharmaceutical composition is within about 80.00% to about 125.00% of an AUC₀₋ₜ₁ of 982 (CV 16.2%) h·ng/ml, after administration of three doses with a dosing interval of 8 hours, wherein each of said three doses corresponds to 100 μg/kg.

In some embodiments, the geometric mean AUC₀₋ₜ₁ of the liquid pharmaceutical composition is within about 80.00% to about 125.00% of an AUC₀₋ₜ₁ of 1664 (CV 17.6%) h·ng/ml, after administration of three doses with a dosing interval of 8 hours, wherein each of said three doses corresponds to 140 μg/kg.

In some embodiments, the geometric mean AUC₀₋ₜ₁ of the liquid pharmaceutical composition is within about 80.00% to about 125.00% of an AUC₀₋ₜ₁ of 2489 (CV 17.5%) h·ng/ml, after administration of three doses with a dosing interval of 8 hours, wherein each of said three doses corresponds to 200 μg/kg.

In some embodiments, the geometric mean AUC₀₋ₜ₁ of the liquid pharmaceutical composition is within about 80.00% to about 125.00% of an AUC₀₋ₜ₁ of 2496 (CV 19.8%) h·ng/ml, after administration of three doses corresponding to 250 μg/kg with a dosing interval of 8 hours.

In some embodiments, the mean AUC₀₋ₜ₂ of the liquid pharmaceutical composition is selected from the group consisting of:
a. within about 80.00% to about 125.00% of an AUC₀₋ₜ₂ of 420 (CV 15.9%) h·ng/ml, after administration of three doses with a dosing interval of 8 hours, wherein each of said three doses corresponds to 50 μg/kg,
b. within about 80.00% to about 125.00% of an AUC₀₋ₜ₂ of 1128 (CV 12.0%) h·ng/mL, after administration of three doses with a dosing interval of 8 hours, wherein each of said three doses corresponds to 100 μg/kg,
c. within about 80.00% to about 125.00% of an AUC₀₋ₜ₂ of 1870 (CV 39.0%) h·ng/ml, after administration of three doses with a dosing interval of 8 hours, wherein each of said three doses corresponds to 140 μg/kg,
d. within about 80.00% to about 125.00% of an AUC₀₋ₜ₂ of 2610 (CV 21.0%) h·ng/ml, after administration of three doses with a dosing interval of 8 hours, wherein each of said three doses corresponds to 200 μg/kg, and
e. within about 80.00% to about 125.00% of an AUC₀₋ₜ₂ of 2354 (CV 25%) h·ng/mL, after administration of three doses with a dosing interval of 8 hours, wherein each of said three doses corresponds to 250 μg/kg.

In some embodiments, the mean AUC₀₋ₜ₂ of the liquid pharmaceutical composition is within about 80.00% to about 125.00% of an AUC₀₋ₜ₂ of 420 (CV 15.9%) h·ng/ml, after administration of three doses with a dosing interval of 8 hours, wherein each of said three doses corresponds to 50 μg/kg.

In some embodiments, the mean AUC₀₋ₜ₂ of the liquid pharmaceutical composition is within about 80.00% to about 125.00% of an AUC₀₋ₜ₂ of 1128 (CV 12.0%) h·ng/ml, after administration of three doses with a dosing interval of 8 hours, wherein each of said three doses corresponds to 100 μg/kg.

In some embodiments, the mean AUC₀₋ₜ₂ of the liquid pharmaceutical composition is within about 80.00% to about 125.00% of an AUC₀₋ₜ₂ of 1870 (CV 39.0%) h·ng/ml, after administration of three doses with a dosing interval of 8 hours, wherein each of said three doses corresponds to 140 μg/kg.

In some embodiments, the mean AUC₀₋ₜ₂ of the liquid pharmaceutical composition is within about 80.00% to about 125.00% of an AUC₀₋ₜ₂ of 2610 (CV 21.0%) h·ng/ml, after administration of three doses with a dosing interval of 8 hours, wherein each of said three doses corresponds to 200 μg/kg.

In some embodiments, the mean AUC₀₋ₜ₂ of the liquid pharmaceutical composition is within about 80.00% to about 125.00% of an AUC₀₋ₜ₂ of 2354 (CV 25%) h·ng/ml, after administration of three doses corresponding to 250 μg/kg with a dosing interval of 8 hours.

In some embodiments, the geometric mean AUC₀₋ₜ₂ of the liquid pharmaceutical composition is selected from the group consisting of:
a. within about 80.00% to about 125.00% of an AUC₀₋ₜ₂ of 416 (CV 15.9%) h·ng/ml, after administration of three doses with a dosing interval of 8 hours, wherein each of said three doses corresponds to 50 μg/kg,
b. within about 80.00% to about 125.00% of an AUC₀₋ₜ₂ of 1121 (CV 12.0%) h·ng/ml, after administration of three doses with a dosing interval of 8
c. within about 80.00% to about 125.00% of an AUC₀₋ₜ₂ of 1761 (CV 39.0%) h·ng/mL, after administration of three doses with a dosing interval of 8 hours, wherein each of said three doses corresponds to 140 μg/kg,
d. within about 80.00% to about 125.00% of an AUC₀₋ₜ₂ of 2559 (CV 21.0%) h·ng/ml, after administration of three doses with a dosing interval of 8 hours, wherein each of said three doses corresponds to 200 μg/kg, and
e. within about 80.00% to about 125.00% of an AUC₀₋ₜ₂ of 2299 (CV 25%) h·ng/ml, after administration of three doses with a dosing interval of 8 hours, wherein each of said three doses corresponds to 250 μg/kg.

In some embodiments, the geometric mean AUC₀₋ₜ₂ of the liquid pharmaceutical composition is within about 80.00% to about 125.00% of an AUC₀₋ₜ₂ of 416 (CV 15.9%) h·ng/mL, after administration of three doses with a dosing interval of 8 hours, wherein each of said three doses corresponds to 50 μg/kg.

In some embodiments, the geometric mean AUC₀₋ₜ₂ of the liquid pharmaceutical composition is within about 80.00% to about 125.00% of an AUC₀₋ₜ₂ of 1121 (CV 12.0%) h·ng/ml, after administration of three doses with a dosing interval of 8 hours, wherein each of said three doses corresponds to 100 μg/kg.

In some embodiments, the geometric mean AUC₀₋ₜ₂ of the liquid pharmaceutical composition is within about 80.00% to about 125.00% of an AUC₀₋ₜ₂ of 1761 (CV 39.0%) h·ng/ml, after administration of three doses with a dosing interval of 8 hours, wherein each of said three doses corresponds to 140 μg/kg.

In some embodiments, the geometric mean AUC₀₋ₜ₂ of the liquid pharmaceutical composition is within about 80.00% to about 125.00% of an AUC₀₋ₜ₂ of 2559 (CV 21.0%) h·ng/ml, after administration of three doses with a dosing interval of 8 hours, wherein each of said three doses corresponds to 200 μg/kg.

In some embodiments, the geometric mean AUC₀₋ₜ₂ of the liquid pharmaceutical composition is within about 80.00% to about 125.00% of an AUC₀₋ₜ₂ of 2299 (CV 25%) h·ng/ml, after administration of three doses corresponding to 250 μg/kg with a dosing interval of 8 hours.

In some embodiments, the mean AUC₀₋ₜ₃ of the liquid pharmaceutical composition is selected from the group consisting of:
a. within about 80.00% to about 125.00% of an AUC₀₋ₜ₃ of 437 (CV 13.7%) h·ng/ml, after administration of three doses with a dosing interval of 8 hours, wherein each of said three doses corresponds to 50 μg/kg,
b. within about 80.00% to about 125.00% of an AUC₀₋ₜ₃ of 1091 (CV 11.1%) h·ng/ml, after administration of three doses with a dosing interval of 8 hours, wherein each of said three doses corresponds to 100 μg/kg,
c. within about 80.00% to about 125.00% of an AUC₀₋ₜ₃ of 1597 (CV 19.2%) h·ng/mL, after administration of three doses with a dosing interval of 8 hours, wherein each of said three doses corresponds to 140 μg/kg,
d. within about 80.00% to about 125.00% of an AUC₀₋ₜ₃ of 2723 (CV 17.7%) h·ng/ml, after administration of three doses with a dosing interval of 8 hours, wherein each of said three doses corresponds to 200 μg/kg, and
e. within about 80.00% to about 125.00% of an AUC₀₋ₜ₃ of 2431 (CV 16.1%) h·ng/ml, after administration of three doses with a dosing interval of 8 hours, wherein each of said three doses corresponds to 250 μg/kg.

In some embodiments, the mean AUC₀₋ₜ₃ of the liquid pharmaceutical composition is within about 80.00% to about 125.00% of an AUC₀₋ₜ₃ of 437 (CV 13.7%) h·ng/ml, after administration of three doses with a dosing interval of 8 hours, wherein each of said three doses corresponds to 50 μg/kg.

In some embodiments, the mean AUC₀₋ₜ₃ of the liquid pharmaceutical composition is within about 80.00% to about 125.00% of an AUC₀₋ₜ₃ of 1091 (CV 11.1%) h·ng/mL, after administration of three doses with a dosing interval of 8 hours, wherein each of said three doses corresponds to 100 μg/kg.

In some embodiments, the mean AUC₀₋ₜ₃ of the liquid pharmaceutical composition is within about 80.00% to about 125.00% of an AUC₀₋ₜ₃ of 1597 (CV 19.2%) h·ng/mL, after administration of three doses with a dosing interval of 8 hours, wherein each of said three doses corresponds to 140 μg/kg.

In some embodiments, the mean AUC₀₋ₜ₃ of the liquid pharmaceutical composition is within about 80.00% to about 125.00% of an AUC₀₋ₜ₃ of 2723 (CV 17.7%) h·ng/ml, after administration of three doses with a dosing interval of 8 hours, wherein each of said three doses corresponds to 200 μg/kg.

In some embodiments, the mean AUC₀₋ₜ₃ of the liquid pharmaceutical composition is within about 80.00% to about 125.00% of an AUC₀₋ₜ₃ of 2431 (CV 16.1%) h·ng/ml, after administration of three doses corresponding to 250 μg/kg with a dosing interval of 8 hours.

In some embodiments, the geometric mean AUC₀₋ₜ₃ of the liquid pharmaceutical composition is selected from the group consisting of:
a. within about 80.00% to about 125.00% of an AUC₀₋ₜ₃ of 433 (CV 13.7%) h·ng/mL, after administration of three doses with a dosing interval of 8 hours, wherein each of said three doses corresponds to 50 μg/kg,
b. within about 80.00% to about 125.00% of an AUC₀₋ₜ₃ of 1085 (CV 11.1%) h·ng/ml, after administration of three doses with a dosing interval of 8 hours, wherein each of said three doses corresponds to 100 μg/kg,
c. within about 80.00% to about 125.00% of an AUC₀₋ₜ₃ of 1570 (CV 19.2%) h·ng/ml, after administration of three doses with a dosing interval of 8 hours, wherein each of said three doses corresponds to 140 μg/kg,
d. within about 80.00% to about 125.00% of an AUC₀₋ₜ₃ of 2687 (CV 17.7%) h·ng/ml, after administration of three doses with a dosing interval of 8 hours, wherein each of said three doses corresponds to 200 μg/kg, and
e. within about 80.00% to about 125.00% of an AUC₀₋ₜ₃ of 2405 (CV 16.1%) h·ng/ml, after administration of three doses with a dosing interval of 8 hours, wherein each of said three doses corresponds to 250 μg/kg.

In some embodiments, the geometric mean AUC₀₋ₜ₃ of the liquid pharmaceutical composition is within about 80.00% to about 125.00% of an AUC₀₋ₜ₃ of 433 (CV 13.7%) h·ng/ml, after administration of three doses with a dosing interval of 8 hours, wherein each of said three doses corresponds to 50 μg/kg.

In some embodiments, the geometric mean AUC₀₋ₜ₃ of the liquid pharmaceutical composition is within about 80.00% to about 125.00% of an AUC₀₋ₜ₃ of 1085 (CV 11.1%) h·ng/ml, after administration of three doses with a dosing interval of 8 hours, wherein each of said three doses corresponds to 100 μg/kg.

In some embodiments, the geometric mean AUC₀₋ₜ₃ of the liquid pharmaceutical composition is within about 80.00% to about 125.00% of an AUC₀₋ₜ₃ of 1570 (CV 19.2%) h·ng/ml, after administration of three doses with a dosing interval of 8 hours, wherein each of said three doses corresponds to 140 μg/kg.

In some embodiments, the geometric mean AUC₀₋ₜ₃ of the liquid pharmaceutical composition is within about 80.00% to about 125.00% of an AUC₀₋ₜ₃ of 2687 (CV 17.7%) h·ng/ml, after administration of three doses with a dosing interval of 8 hours, wherein each of said three doses corresponds to 200 μg/kg.

In some embodiments, the geometric mean AUC₀₋ₜ₃ of the liquid pharmaceutical composition is within about 80.00% to about 125.00% of an AUC₀₋ₜ₃ of 2405 (CV 16.1%) h·ng/ml, after administration of three doses corresponding to 250 μg/kg with a dosing interval of 8 hours.

In some embodiments, the mean Cₘₐₓ₁ of the liquid pharmaceutical composition is selected from the group consisting of:
a. within about 80.00% to about 125.00% of a Cₘₐₓ₁ of 406 (CV 12.3%) ng/ml, after administration of three doses with a dosing interval of 8 hours, wherein each of said three doses corresponds to 50 μg/kg,
b. within about 80.00% to about 125.00% of a Cₘₐₓ₁ of 883 (CV 13.2%) ng/mL, after administration of three doses with a dosing interval of 8 hours, wherein each of said three doses corresponds to 100 μg/kg,
c. within about 80.00% to about 125.00% of a Cₘₐₓ₁ of 1340 (CV 20.7%) ng/ml, after administration of three doses with a dosing interval of 8 hours, wherein each of said three doses corresponds to 140 μg/kg,
d. within about 80.00% to about 125.00% of a Cₘₐₓ₁ of 2190 (CV 16.9%) ng/ml, after administration of three doses with a dosing interval of 8 hours, wherein each of said three doses corresponds to 200 μg/kg, and
e. within about 80.00% to about 125.00% of a Cₘₐₓ₁ of 1960 (CV 26.5%) ng/ml, after administration of three doses with a dosing interval of 8 hours, wherein each of said three doses corresponds to 250 μg/kg.

In some embodiments, the mean Cₘₐₓ₁ of the liquid pharmaceutical composition is within about 80.00% to about 125.00% of a Cₘₐₓ₁ of 406 (CV 12.3%) ng/mL, after administration of three doses with a dosing interval of 8 hours, wherein each of said three doses corresponds to 50 μg/kg.

In some embodiments, the mean Cₘₐₓ₁ of the liquid pharmaceutical composition is within about 80.00% to about 125.00% of a Cₘₐₓ₁ of 883 (CV 13.2%) ng/ml, after administration of three doses with a dosing interval of 8 hours, wherein each of said three doses corresponds to 100 μg/kg.

In some embodiments, the mean Cₘₐₓ₁ of the liquid pharmaceutical composition is within about 80.00% to about 125.00% of a Cₘₐₓ₁ of 1340 (CV 20.7%) ng/ml, after administration of three doses with a dosing interval of 8 hours, wherein each of said three doses corresponds to 140 μg/kg.

In some embodiments, the mean Cₘₐₓ₁ of the liquid pharmaceutical composition is within about 80.00% to about 125.00% of a Cₘₐₓ₁ of 2190 (CV 16.9%) ng/mL, after administration of three doses with a dosing interval of 8 hours, wherein each of said three doses corresponds to 200 μg/kg.

In some embodiments, the mean Cₘₐₓ₁ of the liquid pharmaceutical composition is within about 80.00% to about 125.00% of a Cₘₐₓ₁ of 1960 (CV 26.5%) ng/ml, after administration of three doses corresponding to 250 μg/kg with a dosing interval of 8 hours.

In some embodiments, the geometric mean Cₘₐₓ₁ of the liquid pharmaceutical composition is selected from the group consisting of:
a. within about 80.00% to about 125.00% of a Cₘₐₓ₁ of 403 (CV 12.3%) ng/ml, after administration of three doses with a dosing interval of 8 hours, wherein each of said three doses corresponds to 50 μg/kg,
b. within about 80.00% to about 125.00% of a Cₘₐₓ₁ of 877 (CV 13.2%) ng/ml, after administration of three doses with a dosing interval of 8 hours, wherein each of said three doses corresponds to 100 μg/kg,
c. within about 80.00% to about 125.00% of a Cₘₐₓ₁ of 1310 (CV 20.7%) ng/ml, after administration of three doses with a dosing interval of 8 hours, wherein each of said three doses corresponds to 140 μg/kg,
d. within about 80.00% to about 125.00% of a Cₘₐₓ₁ of 2170 (CV 16.9%) ng/ml, after administration of three doses with a dosing interval of 8 hours, wherein each of said three doses corresponds to 200 μg/kg, and
e. within about 80.00% to about 125.00% of a Cₘₐₓ₁ of 1890 (CV 26.5%) ng/ml, after administration of three doses with a dosing interval of 8 hours, wherein each of said three doses corresponds to 250 μg/kg.

In some embodiments, the geometric mean Cₘₐₓ₁ of the liquid pharmaceutical composition is within about 80.00% to about 125.00% of a Cₘₐₓ₁ of 403 (CV 12.3%) ng/ml, after administration of three doses with a dosing interval of 8 hours, wherein each of said three doses corresponds to 50 μg/kg.

In some embodiments, the geometric mean Cₘₐₓ₁ of the liquid pharmaceutical composition is within about 80.00% to about 125.00% of a Cₘₐₓ₁ of 877 (CV 13.2%) ng/ml, after administration of three doses with a dosing interval of 8 hours, wherein each of said three doses corresponds to 100 μg/kg.

In some embodiments, the geometric mean Cₘₐₓ₁ of the liquid pharmaceutical composition is within about 80.00% to about 125.00% of a Cₘₐₓ₁ of 1310 (CV 20.7%) ng/ml, after administration of three doses with a dosing interval of 8 hours, wherein each of said three doses corresponds to 140 μg/kg.

In some embodiments, the geometric mean Cₘₐₓ₁ of the liquid pharmaceutical composition is within about 80.00% to about 125.00% of a Cₘₐₓ₁ of 2170 (CV 16.9%) ng/ml, after administration of three doses with a dosing interval of 8 hours, wherein each of said three doses corresponds to 200 μg/kg.

In some embodiments, the geometric mean Cₘₐₓ₁ of the liquid pharmaceutical composition is within about 80.00% to about 125.00% of a Cₘₐₓ₁ of 1890 (CV 26.5%) ng/ml, after administration of three doses corresponding to 250 μg/kg with a dosing interval of 8 hours.

In some embodiments, the mean Cₘₐₓ₂ of the liquid pharmaceutical composition is selected from the group consisting of:
a. within about 80.00% to about 125.00% of a Cₘₐₓ₂ of 395 (CV 15.7%) ng/mL, after administration of three doses with a dosing interval of 8 hours, wherein each of said three doses corresponds to 50 μg/kg,
b. within about 80.00% to about 125.00% of a Cₘₐₓ₂ of 977 (CV 12.4%) ng/ml, after administration of three doses with a dosing interval of 8 hours, wherein each of said three doses corresponds to 100 μg/kg,
c. within about 80.00% to about 125.00% of a Cₘₐₓ₂ of 1390 (CV 23.6%) ng/ml, after administration of three doses with a dosing interval of 8 hours, wherein each of said three doses corresponds to 140 μg/kg,
d. within about 80.00% to about 125.00% of a Cₘₐₓ₂ of 2050 (CV 17.6%) ng/ml, after administration of three doses with a dosing interval of 8 hours, wherein each of said three doses corresponds to 200 μg/kg, and
e. within about 80.00% to about 125.00% of a Cₘₐₓ₂ of 1930 (CV 45.4%) ng/ml, after administration of three doses with a dosing interval of 8 hours, wherein each of said three doses corresponds to 250 μg/kg.

In some embodiments, the mean Cₘₐₓ₂ of the liquid pharmaceutical composition is within about 80.00% to about 125.00% of a Cₘₐₓ₂ of 395 (CV 15.7%) ng/ml, after administration of three doses with a dosing interval of 8 hours, wherein each of said three doses corresponds to 50 μg/kg.

In some embodiments, the mean Cₘₐₓ₂ of the liquid pharmaceutical composition is within about 80.00% to about 125.00% of a Cₘₐₓ₂ of 977 (CV 12.4%) ng/ml, after administration of three doses with a dosing interval of 8 hours, wherein each of said three doses corresponds to 100 μg/kg.

In some embodiments, the mean Cₘₐₓ₂ of the liquid pharmaceutical composition is within about 80.00% to about 125.00% of a Cₘₐₓ₂ of 1390 (CV 23.6%) ng/ml, after administration of three doses with a dosing interval of 8 hours, wherein each of said three doses corresponds to 140 μg/kg.

In some embodiments, the mean Cₘₐₓ₂ of the liquid pharmaceutical composition is within about 80.00% to about 125.00% of a Cₘₐₓ₂ of 2050 (CV 17.6%) ng/ml, after administration of three doses with a dosing interval of 8 hours, wherein each of said three doses corresponds to 200 μg/kg.

In some embodiments, the mean Cₘₐₓ₂ of the liquid pharmaceutical composition is within about 80.00% to about 125.00% of a Cₘₐₓ₂ of 1930 (CV 45.4%) ng/ml, after administration of three doses corresponding to 250 μg/kg with a dosing interval of 8 hours.

In some embodiments, the geometric mean Cₘₐₓ₂ of the liquid pharmaceutical composition is selected from the group consisting of:
a. within about 80.00% to about 125.00% of a Cₘₐₓ₂ of 391 (CV 15.7%) ng/mL, after administration of three doses with a dosing interval of 8 hours, wherein each of said three doses corresponds to 50 μg/kg,
b. within about 80.00% to about 125.00% of a Cₘₐₓ₂ of 971 (CV 12.4%) ng/mL, after administration of three doses with a dosing interval of 8 hours, wherein each of said three doses corresponds to 100 μg/kg,
c. within about 80.00% to about 125.00% of a Cₘₐₓ₂ of 1360 (CV 23.6%) ng/ml, after administration of three doses with a dosing interval of 8 hours, wherein each of said three doses corresponds to 140 μg/kg,
d. within about 80.00% to about 125.00% of a Cₘₐₓ₂ of 2020 (CV 17.6%) ng/ml, after administration of three doses with a dosing interval of 8 hours, wherein each of said three doses corresponds to 200 μg/kg, and
e. within about 80.00% to about 125.00% of a Cₘₐₓ₂ of 1800 (CV 45.4%) ng/ml, after administration of three doses with a dosing interval of 8 hours, wherein each of said three doses corresponds to 250 μg/kg.

In some embodiments, the geometric mean Cₘₐₓ₂ of the liquid pharmaceutical composition is within about 80.00% to about 125.00% of a Cₘₐₓ₂ of 391 (CV 15.7%) ng/ml, after administration of three doses with a dosing interval of 8 hours, wherein each of said three doses corresponds to 50 μg/kg.

In some embodiments, the geometric mean Cₘₐₓ₂ of the liquid pharmaceutical composition is within about 80.00% to about 125.00% of a Cₘₐₓ₂ of 971 (CV 12.4%) ng/ml, after administration of three doses with a dosing interval of 8 hours, wherein each of said three doses corresponds to 100 μg/kg.

In some embodiments, the geometric mean Cₘₐₓ₂ of the liquid pharmaceutical composition is within about 80.00% to about 125.00% of a Cₘₐₓ₂ of 1360 (CV 23.6%) ng/ml, after administration of three doses with a dosing interval of 8 hours, wherein each of said three doses corresponds to 140 μg/kg.

In some embodiments, the geometric mean Cₘₐₓ₂ of the liquid pharmaceutical composition is within about 80.00% to about 125.00% of a Cₘₐₓ₂ of 2020 (CV 17.6%) ng/ml, after administration of three doses with a dosing interval of 8 hours, wherein each of said three doses corresponds to 200 μg/kg.

In some embodiments, the geometric mean Cₘₐₓ₂ of the liquid pharmaceutical composition is within about 80.00% to about 125.00% of a Cₘₐₓ₂ of 1800 (CV 45.4%) ng/mL, after administration of three doses corresponding to 250 μg/kg with a dosing interval of 8 hours.

In some embodiments, the mean Cₘₐₓ₃ of the liquid pharmaceutical composition is selected from the group consisting of:
a. within about 80.00% to about 125.00% of a Cₘₐₓ₃ of 398 (CV 12.3%) ng/mL, after administration of three doses with a dosing interval of 8 hours, wherein each of said three doses corresponds to 50 μg/kg,
b. within about 80.00% to about 125.00% of a Cₘₐₓ₃ of 970 (CV 17.7%) ng/ml, after administration of three doses with a dosing interval of 8 hours, wherein each of said three doses corresponds to 100 μg/kg,
c. within about 80.00% to about 125.00% of a Cₘₐₓ₃ of 1240 (CV 18.0%) ng/ml, after administration of three doses with a dosing interval of 8 hours, wherein each of said three doses corresponds to 140 μg/kg,
d. within about 80.00% to about 125.00% of a Cₘₐₓ₃ of 2170 (CV 20.1%) ng/mL, after administration of three doses with a dosing interval of 8 hours, wherein each of said three doses corresponds to 200 μg/kg, and
e. within about 80.00% to about 125.00% of a Cₘₐₓ₃ of 1800 (CV 14.7%) ng/ml, after administration of three doses with a dosing interval of 8 hours, wherein each of said three doses corresponds to 250 μg/kg.

In some embodiments, the mean Cₘₐₓ₃ of the liquid pharmaceutical composition is within about 80.00% to about 125.00% of a Cₘₐₓ₃ of 398 (CV 12.3%) ng/mL, after administration of three doses with a dosing interval of 8 hours, wherein each of said three doses corresponds to 50 μg/kg.

In some embodiments, the mean Cₘₐₓ₃ of the liquid pharmaceutical composition is within about 80.00% to about 125.00% of a Cₘₐₓ₃ of 970 (CV 17.7%) ng/ml, after administration of three doses with a dosing interval of 8 hours, wherein each of said three doses corresponds to 100 μg/kg.

In some embodiments, the mean Cₘₐₓ₃ of the liquid pharmaceutical composition is within about 80.00% to about 125.00% of a Cₘₐₓ₃ of 1240 (CV 18.0%) ng/ml, after administration of three doses with a dosing interval of 8 hours, wherein each of said three doses corresponds to 140 μg/kg.

In some embodiments, the mean Cₘₐₓ₃ of the liquid pharmaceutical composition is within about 80.00% to about 125.00% of a Cₘₐₓ₃ of 2170 (CV 20.1%) ng/mL, after administration of three doses with a dosing interval of 8 hours, wherein each of said three doses corresponds to 200 μg/kg.

In some embodiments, the mean Cₘₐₓ₃ of the liquid pharmaceutical composition is within about 80.00% to about 125.00% of a Cₘₐₓ₃ of 1800 (CV 14.7%) ng/mL, after administration of three doses corresponding to 250 μg/kg with a dosing interval of 8 hours.

In some embodiments, the geometric mean Cₘₐₓ₃ of the liquid pharmaceutical composition is selected from the group consisting of:
a. within about 80.00% to about 125.00% of a Cₘₐₓ₃ of 396 (CV 12.3%) ng/ml, after administration of three doses with a dosing interval of 8 hours, wherein each of said three doses corresponds to 50 μg/kg,
b. within about 80.00% to about 125.00% of a Cₘₐₓ₃ of 958 (CV 17.7%) ng/mL, after administration of three doses with a dosing interval of 8 hours, wherein each of said three doses corresponds to 100 μg/kg,
c. within about 80.00% to about 125.00% of a Cₘₐₓ₃ of 1220 (CV 18.0%) ng/ml, after administration of three doses with a dosing interval of 8 hours, wherein each of said three doses corresponds to 140 μg/kg,
d. within about 80.00% to about 125.00% of a Cₘₐₓ₃ of 2130 (CV 20.1%) ng/mL, after administration of three doses with a dosing interval of 8 hours, wherein each of said three doses corresponds to 200 μg/kg, and
e. within about 80.00% to about 125.00% of a Cₘₐₓ₃ of 1780 (CV 14.7%) ng/mL, after administration of three doses with a dosing interval of 8 hours, wherein each of said three doses corresponds to 250 μg/kg.

In some embodiments, the geometric mean Cₘₐₓ₃ of the liquid pharmaceutical composition is within about 80.00% to about 125.00% of a Cₘₐₓ₃ of 396 (CV 12.3%) ng/ml, after administration of three doses with a dosing interval of 8 hours, wherein each of said three doses corresponds to 50 μg/kg.

In some embodiments, the geometric mean Cₘₐₓ₃ of the liquid pharmaceutical composition is within about 80.00% to about 125.00% of a Cₘₐₓ₃ of 958 (CV 17.7%) ng/ml, after administration of three doses with a dosing interval of 8 hours, wherein each of said three doses corresponds to 100 μg/kg.

In some embodiments, the geometric mean Cₘₐₓ₃ of the liquid pharmaceutical composition is within about 80.00% to about 125.00% of a Cₘₐₓ₃ of 1220 (CV 18.0%) ng/mL, after administration of three doses with a dosing interval of 8 hours, wherein each of said three doses corresponds to 140 μg/kg.

In some embodiments, the geometric mean Cₘₐₓ₃ of the liquid pharmaceutical composition is within about 80.00% to about 125.00% of a Cₘₐₓ₃ of 2130 (CV 20.1%) ng/ml, after administration of three doses with a dosing interval of 8 hours, wherein each of said three doses corresponds to 200 μg/kg.

In some embodiments, the geometric mean Cₘₐₓ₃ of the liquid pharmaceutical composition is within about 80.00% to about 125.00% of a Cₘₐₓ₃ of 1780 (CV 14.7%) ng/ml, after administration of three doses corresponding to 250 μg/kg with a dosing interval of 8 hours.

In some embodiments, each of said three doses is administered as a 30-minute i.v. infusion to a human subject.

In some embodiments, the AUC_{0-24 hrs}, AUC₀₋ₜ₁, AUC₀₋ₜ₂, AUC₀₋ₜ₃, Cₘₐₓ₁, Cₘₐₓ₂ and/or Cₘₐₓ₃ is measured after administration of a single dose to a human subject, such as a healthy male Caucasian subject.

In some embodiments, the present disclosure relates to a method of achieving a t_{1/2}, AUC_{0-24 hrs}, AUC₀₋ₜ₁, AUC₀₋ₜ₂, AUC₀₋ₜ₃, Cₘₐₓ₁, Cₘₐₓ₂ and/or Cₘₐₓ₃ as disclosed herein by administering the liquid pharmaceutical composition to a human subject as a 1-120 minutes intravenous (i.v.) infusion.

In some embodiments, the liquid pharmaceutical composition is diluted prior to administration.

In some embodiments, the liquid pharmaceutical composition is diluted based on the human subjects body weight and/or to maintain the same volume for infusion.

In some embodiments, the liquid pharmaceutical composition is administered by i.v. infusion with a flow rate of about 0.5 to 10 mL/min., such as of 0.5 to 10 mL/min., such as of 0.5 to 5 mL/min., such as of 0.5 to 4 mL/min., such as of 0.5 to 3 mL/min., such as of 1 to 2 mL/min., such as of about 1.5 mL/min.

In some embodiments, the liquid pharmaceutical composition is administered by i.v. infusion with a flow rate of 1.5 mL/min.

In some embodiments, the present disclosure relates to the liquid pharmaceutical composition as disclosed herein for use in the treatment of an ischemic condition and/or an inflammatory condition.

In some embodiments, the present disclosure relates to the liquid pharmaceutical composition as disclosed herein for use in the treatment of a cardiovascular disease.

In some embodiments, the present disclosure relates to the liquid pharmaceutical composition as disclosed herein for use in the treatment of a heart disease. In some embodiments, the heart disease is characterised by having a pathology that is driven by acute exacerbated inflammation. In some embodiments, acute exacerbated inflammation is the main driver of heart disease pathology.

Cardiovascular disease is a class of diseases that involve the heart or blood vessels.

Cardiovascular diseases include coronary artery diseases, such as angina and myocardial infarction (commonly known as a heart attack). Other cardiovascular diseases include stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis.

The underlying mechanisms vary depending on the disease. Coronary artery disease, stroke (cerebrovascular disease) and peripheral artery disease involve atherosclerosis, and hence these can be classified as atherosclerotic cardiovascular disease.

In some embodiments, the present disclosure relates to the liquid pharmaceutical composition as disclosed herein for use in the treatment of a cardiovascular disease selected from the group consisting of coronary artery diseases, such as angina and myocardial infarction (commonly known as a heart attack), stroke, heart failure, hypertensive heart disease, rheumatic heart disease, cardiomyopathy, abnormal heart rhythms, congenital heart disease, valvular heart disease, carditis, aortic aneurysms, peripheral artery disease, vascular disease, thromboembolic disease, and venous thrombosis.

In some embodiments, the present disclosure relates to the liquid pharmaceutical composition as disclosed herein for use in the treatment of an ischemic condition and/or an inflammatory condition during heart surgery and/or aorta surgery, such as open heart surgery.

In some embodiments, the present disclosure relates to the liquid pharmaceutical composition as disclosed herein for use in the treatment of myocardial ischemic condition and/or reperfusion injury of the heart.

In some embodiments, the present disclosure relates to the liquid pharmaceutical composition as disclosed herein for use in the treatment of coronary artery disease.

In some embodiments, the present disclosure relates to the liquid pharmaceutical composition as disclosed herein for use in the treatment of a coronary artery disease selected from the group consisting of angina and myocardial infarction; stroke, heart failure; peripheral artery disease; thromboembolic disease; and venous thrombosis.

In some embodiments, the present disclosure relates to the liquid pharmaceutical composition as disclosed herein for use in treatment of myocardial infarction. In some embodiments, the present disclosure relates to the liquid pharmaceutical composition as disclosed herein for use in treatment of acute myocardial infarction.

In some embodiments, the present disclosure relates to the liquid pharmaceutical composition as disclosed herein for use in treatment of an ischemic condition and/or a reperfusion injury selected from the group consisting of myocardial infarction due to myocardial ischemia/reperfusion, myocardial infarction due to myocardial ischemia, myocarditis, sepsis-induced cardiomyopathy, and sepsis-induced myocardial inflammation.

In some embodiments, the present disclosure relates to the liquid pharmaceutical composition as disclosed herein for use in treatment of myocardial infarction due to myocardial ischemia or myocardial infarction due to myocardial ischemia/reperfusion.

Myocardial infarction (MI) in general can be classified from Type 1 to Type 5 MI based on the etiology and pathogenesis. Type 1 MI is due to acute coronary atherothrombotic myocardial injury with plaque rupture. Most patients with ST-segment elevation MI (STEMI) and many with non-ST-segment elevation MI (NSTEMI) comprise this category. Type 2 MI is the most common type of MI encountered in clinical settings in which is there is demand-supply mismatch resulting in myocardial ischemia. This demand supply mismatch can be due to multiple reasons including but not limited to presence of a fixed stable coronary obstruction, tachycardia, hypoxia or stress. However, the presence of fixed coronary obstruction is not necessary. Other potential etiologies include coronary asospasm, coronary embolus, and spontaneous coronary artery dissection (SCAD). Sudden cardiac death patients who succumb before any troponin elevation comprise Type 3 MI. Types 4 and 5 MIs are related to coronary revascularization procedures like Percutaneous Coronry Intervention (PCI) or Coronary artery Bypass Grfting (CABG).

In some embodiments, the present disclosure relates to the liquid pharmaceutical composition as disclosed herein for use in treatment of myocardial infarction Type 1 to Type 5. In some embodiments, the present disclosure relates to the liquid pharmaceutical composition as disclosed herein for use in treatment of acute myocardial infarction (AMI).

In some embodiments, the present disclosure relates to the liquid pharmaceutical composition as disclosed herein for use in the treatment of ST-segment elevation myocardial infarction (STEMI) or non-ST-segment elevation myocardial infarction (NSTEMI).

In some embodiments, the present disclosure relates to the liquid pharmaceutical composition as disclosed herein for use in treatment of ST-elevation myocardial infarction (STEMI).

An ST-elevation myocardial infarction occurs from occlusion of one or more of the coronary arteries that supply the heart with blood. The cause of this abrupt disruption of blood flow is usually plaque rupture, erosion, fissuring or dissection of coronary arteries that results in an obstructing thrombus. The major risk factors for ST-elevation myocardial infarction are dyslipidemia, diabetes mellitus, hypertension, smoking, and family history of coronary artery disease. An acute ST-elevation myocardial infarction (STEMI) is an event in which transmural myocardial ischemia results in myocardial injury or necrosis.

### EXAMPLES

In the following Examples, reference to ‘Peptide 1’ is identified herein as AMVSEFLKQAWFIENEEQEYVQTLKSSKGGPGSAVSPYPTFNPSSDV (SEQ ID NO: 6 (Annexin A1 2-48 V24L)), with a C-terminal amidation.

### General Method of Analysis Used in Examples 1-3

Peptide 1 in blood samples collected from animals used in the studies (rat and dog) has been measured by an LC-MS/MS method. Analysis of samples collected in the pharmacokinetic study in rats was performed prior to validation of the method. Prior to analysis of samples from the GLP toxicology studies, the method was validated according to GLP to ensure that it is selective, accurate, reproducible, and able to produce reliable results. The method was validated over the range from 0.075 to 25 μg/mL Peptide 1.

### Example 1: PK Parameters after Single Administration of Peptide 1 in Rats

The objective of the study was to assess the pharmacokinetic profile of Peptide 1 when administered once by intravenous injection to male Wistar (CRL: WI(Han)) rats at 0.1, 1.0 and 10.0 mg/kg. The number of rats per group was respectively 6, 6 and 8. Blood samples were collected from 3 animals per group at 2, 5, 10, 20, 40 and 60 minutes after dosing; for the animals receiving 10.0 mg/kg, additional samples were taken from 2 animals at 90 and 120 minutes after dosing. The pharmacokinetics were characterised using mean plasma concentration vs. time data. The pharmacokinetic parameters were estimated using Phoenix WinNonlin Version 6.3 pharmacokinetic software.

Peptide 1 was quantifiable in all plasma samples in the study. 26 plasma samples (samples in Group 1 (0.1 mg/kg) obtained 2 minutes post dosing, in Group 2 (1 mg/kg) 2 to 10 minutes post dosing and in Group 3 (10 mg/kg) 2 to 40 minutes post dosing) had concentrations of Peptide 1 above the upper limit of quantification for the bioanalytical method. As a consequence, plasma concentration of Peptide 1 was extrapolated. Plasma samples obtained 60 minutes post dosing in Group 1 had plasma concentration of Peptide 1 below the limit of quantification.

TABLE 1
PK parameters after single administration of Peptide 1 in male rats

| | | Initial Elimination Phase | | | Terminal Elimination Phase | | |
|---|---|---|---|---|---|---|---|
| | | Route | | | | | |
| | | IV | | | IV | | |
| | | Dose (mg/kg) | | | | | |
| | | 0.1 | 1.0 | 10 | 0.1 | 1.0 | 10 |
| Parameter | units | Males | Males | Males | Males | Males | Males |
| C0 | ng/mL | 1790 | 32500 | 68900 | | | |
| T½ | (min) | 3.49 | 3.34 | 7.64 | 18.8 | 10.0 | 10.2 |
| AUC_{Last} | ng · min/mL | 9890 | 124000 | 885000 | | | |
| AUC_{Inf} | mL/h/kg | | | | 10400 | 124000 | 903000 |
| CL | mL/min/kg | 10.0 | 8.08 | 11.1 | 9.66 | 8.05 | 11.1 |
| Vz | mL/kg | 50.5 | 38.9 | 122 | 261 | 116 | 162 |

As shown in Table 1, profiles were generally consistent with intravenous dosing divided into and initial and a terminal elimination phase. The half-life was longer in the terminal elimination phase (range approximately 10 to 19 min) as compared to the initial phase (range app 3 to 8 min). The volume of distribution was higher in the terminal phase (range 116 to 261 mL/kg) as compared to the initial phase (range 39 to 122 mL/kg). The clearance was comparable between the two phases range 8 to 11 mL/min/kg. The exposure based on CO and AUC increased with the increase in dose.

### Example 2: Repeat Dose IV Study with Toxicokinetic Evaluation on Day 1 and Day 14 in Rats

In the 14-day intravenous toxicity study in Wistar rats (see study description below), samples were collected from 3 male and 3 female animals per group dosed at 0 (Control), 1, 3 or 10 mg/kg/day at 2, 5, 10, 20 and 40 minutes after dosing on Days 1 and 14. Samples were analysed for levels of Peptide 1.

Peptide 1 was not detected in control animals. In Group 2 (1 mg/kg), C(0) was calculated in the range 9.96-11.9 μg/mL and AUC_{inf} in the range 55.1-61.6 min*μg/mL. In Group 3 (3 mg/kg), C(0) was calculated in the range 23.0-41.9 μg/mL and AUC_{inf} in the range 137-203 min*μg/mL. In Group 4 (10 mg/kg), C(0) was calculated in the range 92.3-138 μg/mL and AUC_{inf} in the range 548-605 min*μg/mL.

TABLE 2
TK parameters following repeated IV administration of Peptide 1 in rats

| Dose level | | | C0 | T_{1/2} | AUC_{0-∞} | Cl | Vz |
|---|---|---|---|---|---|---|---|
| (mg/kg) | | | (ug/mL) | (min) | (ug · min/mL) | (mL/min/kg) | (mL/kg) |
| 1.0 | male | Day 1 | 9.96 | 8.40 | 55.6 | 18.0 | 218 |
| | female | Day 1 | 11.9 | 7.33 | 55.1 | 18.1 | 192 |
| | male | Day 14 | 10.3 | 7.66 | 58.0 | 17.2 | 190 |
| | female | Day 14 | 10.9 | 7.11 | 61.6 | 16.2 | 167 |
| 3.0 | male | Day 1 | 23.0 | 7.00 | 137 | 21.9 | 221 |
| | female | Day 1 | 30.6 | 8.00 | 172 | 17.4 | 201 |
| | male | Day 14 | 36.7 | 6.67 | 161 | 18.7 | 180 |
| | female | Day 14 | 41.9 | 6.85 | 203 | 14.8 | 146 |
| 10 | male | Day 1 | 104 | 7.80 | 552 | 18.1 | 204 |
| | female | Day 1 | 127 | 7.16 | 574 | 17.4 | 180 |
| | male | Day 14 | 92.3 | 7.31 | 548 | 18.2 | 192 |
| | female | Day 14 | 138 | 7.04 | 605 | 16.5 | 168 |

As shown in Table 2, the elimination half-life was estimated to about 7-8 min and the clearance about 18 mL/min/kg for all groups.

As shown in Table 2, the exposure (C(0) and AUC_{inf}) generally appeared to be slightly higher for females than for males especially when based on C(0). However, the difference was small and might be coincidental. The exposure (C(0) and AUC_{inf}) appeared to increase proportional to dose level from 1 mg/kg to 10 mg/kg. The exposure (C(0) and AUC_{inf}) in Group 3 (3 mg/kg) appeared to be higher on Day 14 compared to Day 1. However, the samples from Group 3 on Day 1 were analysed after 3 freeze/thaw cycles, which according to the bioanalytical validation would lead to a 15% bias. The apparent accumulation was therefore most likely due to instability of the samples from Day 1. In Group 2 (1 mg/kg) and Group 4 (10 mg/kg), the exposure was very similar on Day 1 and Day 14.

### Example 3: Repeat IV Dose Study with Toxicokinetic Evaluation on Day 1 and 14 in Dogs

In the 14-day intravenous toxicity study in beagle dogs (see study description below), samples were collected from 3 male and 3 female animals per group dosed at 0 (Control), 1, 3 or 10 mg/kg/day at 2, 5, 10, 20 and 40 minutes after dosing on Days 1 and 14. Samples were analysed for levels of Peptide 1.

Peptide 1 was not detected in control animals. In Group 2 (1 mg/kg), C(0) was calculated in the range 4.58-8.58 μg/mL and AUC_{inf} in the range 27.0-37.2 min*μg/mL. In Group 3 (3 mg/kg), C(0) was calculated in the range 12.5-20.0 μg/mL and AUC_{inf} in the range 85.2-118 min*μg/mL. In Group 4 (10 mg/kg), C(0) was calculated in the range 26.4-58.3 μg/mL and AUC_{inf} in the range 236-281 min*μg/mL.

TABLE 3
TK parameters following repeated IV administration of Peptide 1 in dogs

| Dose level | | | C0 | T_{1/2} | AUC_{0-∞} | Cl | Vz |
|---|---|---|---|---|---|---|---|
| (mg/kg) | | | (ug/mL) | (min) | (ug · min/mL) | (mL/min/kg) | (mL/kg) |
| 1.0 | male | Day 1 | 4.58 | 4.68 | 27.0 | 37.1 | 251 |
| | female | Day 1 | 6.99 | 4.83 | 32.2 | 33.1 | 232 |
| | male | Day 14 | 6.59 | 6.59 | 33.0 | 31.0 | 297 |
| | female | Day 14 | 8.58 | 4.66 | 37.2 | 27.4 | 183 |
| 3.0 | male | Day 1 | 12.5 | 6.01 | 85.2 | 35.9 | 318 |
| | female | Day 1 | 16.1 | 5.70 | 102 | 30.1 | 248 |
| | male | Day 14 | 19.9 | 6.05 | 111 | 27.4 | 238 |
| | female | Day 14 | 20.0 | 6.13 | 118 | 25.9 | 232 |
| 10 | male | Day 1 | 26.4 | 5.09 | 236 | 42.7 | 318 |
| | female | Day 1 | 53.4 | 5.41 | 273 | 37.1 | 288 |
| | male | Day 14 | 51.4 | 6.00 | 281 | 36.6 | 316 |
| | female | Day 14 | 58.3 | 5.90 | 274 | 36.7 | 312 |

As shown in Table 3, the elimination half-life was estimated to about 4.66 to 6.59 min and the clearance in the range of 27.4 to 42.7 mL/min/kg for all groups. Generally, the exposure (C(0) and AUC_{inf}) appeared to be slightly higher for females than for males.

The exposure (C(0) and AUC_{inf}) appeared to increase proportional to dose level from 1 mg/kg to 10 mg/kg.

It can be concluded from Examples 1-3, that the pharmacokinetic results from the studies performed in rats and dogs are consistent and in general show a rapid clearance of Peptide 1 from the systemic circulation; exposure seems to increase proportional to the dose administered and there is little difference in exposure following repeated doses compared to single doses suggesting no accumulation over time. At the dosages administered (expressed as mg/kg), the exposure in rats is higher than that in dogs.

### Example 4: Clinical Test in Humans-Single Dose

### Study Design

A total of 8 healthy subjects were included in each group. Group 1 received 25 μg/kg of Peptide 1, Group 2 received 50 μg/kg of Peptide 1, Group 3 received 100 μg/kg of Peptide 1, Group 4 received 150 μg/kg of Peptide 1, Group 5 received 200 μg/kg of Peptide 1 and Group 7 received 400 μg/kg of Peptide 1.

The tested liquid pharmaceutical composition has a pH of 8.2-8.4, and comprises the following:
i) 5 mg/ml of Peptide 1,
ii) 4% (w/w) mannitol,
iii) 1% (w/w) sucrose,
iv) 0.01% (w/w) Polysorbate-80,
v) 10 mM glycyl-glycine, and
vi) sterile water for injection.

In these groups, 6 subjects received the active treatment (i.e. a single Peptide 1 30-min intravenous (i.v.) infusion corresponding to the above doses) and 2 received the placebo. In order to prevent any anticipated unblinding, the subjects under active treatment were recorded by the bioanalytical laboratory.

Blood samples were collected for PK purpose for Peptide 1 determinations on Day 1 at the following time points: pre-dose, then, 10 min, 20 min, 30 min (30 min=0.5), 0.75, 1.0, 1.5, 2.0, 2.5, 3.0, 4.0, 6.0, 8.0, 12.0, 16.0 and 24.0 h after the infusion start.

For the interim analysis, only plasma concentrations of Peptide 1 was considered.

### Pharmacokinetics Analyses

Venous whole blood samples were collected from all subjects.

Plasma samples were analyzed for Peptide 1 using validated Liquid chromatography—Tandem Mass spectrometry (LC-MS/MS) methods by Charles River Laboratories (CRL).

Derivation of PK parameters were carried out by PhinC Development using Phoenix WinNonlin® (Version Phoenix 8.1—Certara—Princeton—USA). A non-compartmental analysis (NCA) approach was chosen.

For the calculation of the PK parameters and characteristics the following rules were applied:
All the plasma concentrations validated by the bioanalytical laboratory and provided to the pharmacokineticist were used for the PK analysis.
The theoretical PK blood sampling time points were used.
At time points in the lag-time between time zero and the first concentration equal or above limit of quantification (LOQ), concentrations below the LOQ (BLQ) were set to zero (0).
Concentrations BLQ between 2 concentrations equal or above LOQ were considered as missing.
Trailing concentrations (at time point between time of last measurable concentrations and time of last blood sample) BLQ were not used in calculations.
For concentration above the upper limit of quantification and reported as above limit of quantification (ALQ) in the final plasma concentration tables, ALQ was considered as missing for the PK analysis.
Not reported (NR) concentration was excluded from the PK analysis.
For the first administration, if pre-dose concentration was less than or equal to 5% of Cmax value in a PK profile, the subject's data was included in all PK measurements and calculations without any adjustments. If the pre-dose value was greater than 5% of Cmax, the subject was dropped from all statistical evaluations.

The following PK parameters of Peptide 1, were derived for each subject receiving the active treatment on Day 1:
Cmax: The observed maximum concentration in plasma measured in a subject after dosing identified by inspection of the plasma drug concentration versus (vs.) time data by WinNonlin®.
tmax: The time at which Cmax was apparent, identified by inspection of the plasma drug concentration vs. time data by Phoenix WinNonlin®.
AUC₀₋₂₄: The area under the concentration-time curve from time zero (pre-dose) to 24 h was calculated using a linear trapezoidal method. AUC₀₋₂₄ was only calculated if there were at least 3 concentrations above LOQ.
AUC₀₋ₜ: The area under the concentration-time curve from time zero (pre-dose) to the time of last quantifiable concentration was calculated using a linear trapezoidal method. The AUC was only calculated if there were at least 3 concentrations above LOQ.
kₑ: The terminal plasma elimination rate-constant was estimated from log-linear regression analysis of the terminal phase of the plasma concentration-time profile. The number of points included in the terminal phase was determined by visual inspection of the semi-log plots of the plasma concentration-time profiles. A minimum of 3 data points, including the last measured data point and excluding Cmax, should be available for the regression. The correlation coefficient for the goodness of the fit of the regression line through the data points (r²) had to be 0.80 or higher, for the value to be considered reliable. No determination of derived parameters (t1/2, AUC extrapolation, AUC0-∞, CL and Vd) was performed with unreliable elimination rate constants (hence, considered as not calculable—NC).
AUC_{0-∞}: The AUC from time 0 to infinity was calculated as AUC_{0-∞}=AUC₀₋ₜ+AUC_{t-∞}, where AUC₀₋ₜ is the area under the concentration-time curve from time zero (pre dose) to the time of last quantifiable concentration calculated using a linear trapezoidal method and AUC_{t-∞}=Ct/kₑ, where Ct is the measured concentration at time of the last quantifiable concentration t. The AUC was only calculated if there were at least 3 concentrations above LOQ. The extrapolated part of AUC_{0-∞} must be <20% for the value be considered as reliable. If it exceeded 20%, AUC_{0-∞} was flagged.
t_{1/2}: The apparent terminal elimination half-life was calculated as In2/kₑ, where kₑ is the elimination rate constant as defined above.
CL: The volume of the central compartment cleared of drug per unit time was estimated, for Peptide 1 only, using the formula: CL=Dose/AUC_{0-∞}
Vd: The volume of distribution based on the terminal elimination phase, was estimated, for Peptide 1 only, using the formula: Vd=CL/kₑ

Descriptive statistics for the concentrations were presented as number of observations (N), mean, standard deviation (SD) and were calculated if at least ⅔ of the concentrations per time-point were non-missing. For descriptive statistics calculations, concentration BLQ were set to zero (0) before the first concentration equal or above LOQ and considered as missing after.

Descriptive statistics of the PK parameters were presented as N, mean, SD, coefficient of variation (CV %), median, geometric mean (GM), minimum (Min) and maximum (Max), and were calculated if N≥3.

### Main Pharmacokinetics Results

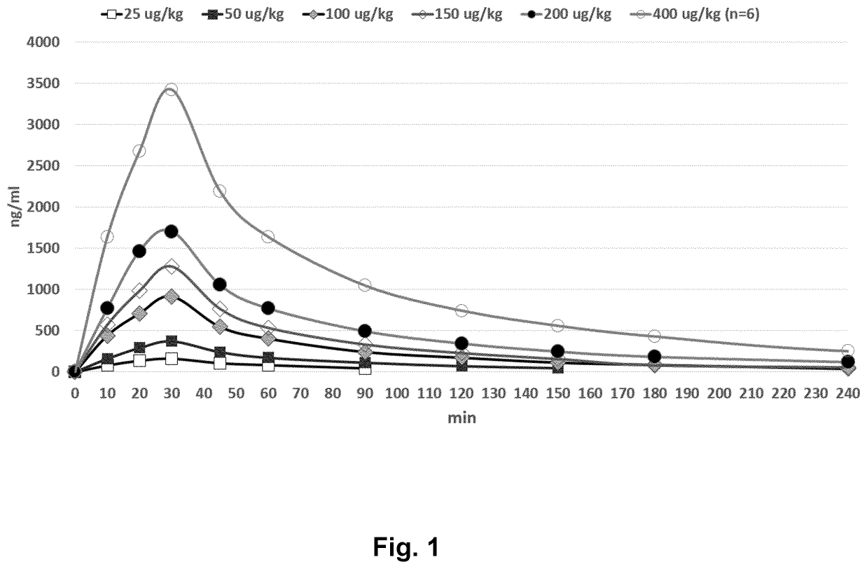

Mean PK profiles of Peptide 1 by dose level are shown in FIG. 1. Main data and descriptive statistics of subjects in Group 1 (25 μg/kg), Group 2 (50 μg/kg), Group 3 (100 μg/kg), Group 4 (150 μg/kg), Group 5 (200 μg/kg) and Group 7 (400 μg/kg) are shown in Table 4A and 4B.

TABLE 4A
Summary of main PK parameters by dose level (25 μg/kg, 50 μg/kg,
100 μg/kg and 150 μg/kg of Peptide 1).

| Dose level | | tₘₐₓ# | Cₘₐₓ | AUC₀₋₂₄ | AUC₀₋ₜ | AUC_{0-inf} | t_{1/2} | CL | Vd |
|---|---|---|---|---|---|---|---|---|---|
| (μg/kg) | | (h) | (ng/mL) | (h*ng/mL) | (h*ng/mL) | (h*ng/mL) | (h) | (L/h/kg) | (L/kg) |
| 25 | N | 6 | 6 | 4 | 6 | 4 | 4 | 4 | 4 |
| | Mean | 0.50 | 162 | 211.21 | 132.50 | 211.22 | 0.79 | 0.12 | 0.14 |
| | SD | 0.00 | 19.1 | 5.55 | 25.71 | 5.55 | 0.13 | 0.00 | 0.02 |
| | CV % | 0.50-0.50 | 11.7 | 2.6 | 19.4 | 2.6 | 16.4 | 2.6 | 16.8 |
| | GM | 0.50 | 161 | 211.15 | 130.30 | 211.17 | 0.79 | 0.12 | 0.13 |
| 50 | N | 6 | 6 | 6 | 6 | 6 | 6 | 6 | 6 |
| | Mean | 0.50 | 375 | 463.63 | 378.17 | 463.50 | 0.92 | 0.11 | 0.14 |
| | SD | 0.00 | 57.1 | 87.19 | 84.81 | 86.89 | 0.24 | 0.02 | 0.03 |
| | CV % | 0.50-0.50 | 15.3 | 18.8 | 22.4 | 18.7 | 25.7 | 20.3 | 18.8 |
| | GM | 0.50 | 371 | 456.55 | 369.29 | 456.47 | 0.90 | 0.11 | 0.14 |
| 100 | N | 6 | 6 | 6 | 6 | 6 | 6 | 6 | 6 |
| | Mean | 0.50 | 916 | 1107.53 | 1001.13 | 1107.51 | 1.15 | 0.09 | 0.15 |
| | SD | 0.00 | 192 | 148.22 | 151.76 | 148.14 | 0.23 | 0.01 | 0.02 |
| | CV % | 0.50-0.50 | 21.0 | 13.4 | 15.2 | 13.4 | 20.2 | 13.6 | 14.8 |
| | GM | 0.50 | 899 | 1099.20 | 991.28 | 1099.18 | 1.13 | 0.09 | 0.15 |
| 150 | N | 6 | 6 | 6 | 6 | 6 | 6 | 6 | 6 |
| | Mean | 0.50 | 1280 | 1521.23 | 1399.28 | 1521.30 | 1.15 | 0.10 | 0.17 |
| | SD | 0.00 | 297 | 344.47 | 307.47 | 344.35 | 0.18 | 0.02 | 0.04 |
| | CV % | 0.50-0.50 | 23.3 | 22.6 | 22.0 | 22.6 | 16.1 | 22.0 | 23.5 |
| | GM | 0.50 | 1250 | 1489.48 | 1371.37 | 1489.59 | 1.13 | 0.10 | 0.16 |

#Median and Min-Max instead of Mean and CV %.

TABLE 4B
Summary of main PK parameters by dose level (200 μg/kg and 400 μg/kg of Peptide 1).

| Dose level | | tₘₐₓ# | Cₘₐₓ | AUC₀₋₂₄ | AUC₀₋ₜ | AUC_{0-inf} | t_{1/2} | CL | Vd |
|---|---|---|---|---|---|---|---|---|---|
| (μg/kg) | | (h) | (ng/mL) | (h*ng/mL) | (h*ng/mL) | (h*ng/mL) | (h) | (L/h/kg) | (L/kg) |
| 200 | N | 6 | 6 | 6 | 6 | 6 | 6 | 6 | 6 |
| | Mean | 0.50 | 1700 | 2277.62 | 2119.03 | 2277.73 | 1.45 | 0.09 | 0.18 |
| | SD | 0.00 | 74.2 | 70.73 | 110.33 | 70.65 | 0.17 | 0.00 | 0.02 |
| | CV % | 0.50-0.50 | 4.4 | 3.1 | 5.2 | 3.1 | 11.5 | 3.2 | 10.2 |
| | GM | 0.50 | 1700 | 2276.70 | 2116.61 | 2276.80 | 1.44 | 0.09 | 0.18 |
| 400 | N | 6 | 6 | 6 | 6 | 6 | 6 | 6 | 6 |
| | Mean | 0.50 | 3420 | 4871.87 | 4718.68 | 4872.56 | 1.64 | 0.08 | 0.19 |
| | SD | 0.00 | 364 | 554.00 | 558.93 | 554.10 | 0.23 | 0.01 | 0.02 |
| | CV % | 0.50-0.50 | 10.7 | 11.4 | 11.8 | 11.4 | 14.3 | 12.4 | 9.8 |
| | GM | 0.50 | 3400 | 4844.14 | 4689.49 | 4844.83 | 1.62 | 0.08 | 0.19 |

#Median and Min-Max instead of Mean and CV %.

### Conclusion:

Following a single 30-min intravenous infusion of Peptide 1 in the dose ranges from 15-400 μg/kg:
In all subjects, Peptide 1 pre-dose concentrations were BLOQ and the first concentrations were measurable 0.16 hour after the start of infusion. At the highest dose, concentrations were measurable in all subjects up to at least 6 hours after the start of infusion. At the lowest dose, concentrations were measurable in all subjects up to at least 1 hour after start of the infusion.
After a single 30-min intravenous infusion, concentrations increased up to Cₘₐₓ with tₘₐₓ observed at 0.5 hour (time of the end of infusion) for all subjects. Then concentrations decreased.
Exposure parameters (Cₘₐₓ and AUCs) were consistent between subjects with low variability (400 μg/mg CV % for Cₘₐₓ and AUCs was below 12%; for 25 μg/kg mg CV % for Cₘₐₓ and AUCs was below 20%).
Elimination phase could be determined for all subjects, with GM t_{1/2}, ranging from about 0.8 hour to about 1.6 hours at all dose levels.
Clearance and volume of distribution were consistent between all subjects within a given group and between groups, with values at the highest dose around 0.08 L/h/kg and 0.19 L/kg, respectively.
The exposure of Peptide 1 increased with Peptide 1 dose level. Exposure increased with dose with Cₘₐₓ going from (mean per group) 162 ng/ml at the 25 μg/kg dose to 3,420 ng/ml at the 400 μg/kg dose. (Between 100 μg/kg and 400 μg/kg, exposure parameters (Cₘₐₓ and AUCs) increased by about 4.3-fold when the dose increased by 4-fold. Between 150 μg/kg and 400 μg/kg, exposure parameters (Cₘₐₓ and AUCs) increased by around 3.2 fold when the dose increased by 2.7-fold. Between 200 μg/kg and 400 μg/kg, exposure parameters (Cₘₐₓ and AUCs) increased by around 2-fold when the dose increased by 2 fold.)

### Example 5: Clinical Test in Humans—Multiple Doses

A total of 8 healthy subjects are included in each group. Group 1B receives 25 μg/kg of Peptide 1 per intravenous (i.v.) infusion, Group 2B receives 50 μg/kg of Peptide 1 per i.v. infusion, Group 3B receives 100 μg/kg of Peptide 1 per i.v. infusion, Group 4B receives 150 μg/kg of Peptide 1 per i.v. infusion, Group 5B receives 200 μg/kg of Peptide 1 per i.v. infusion and Group 7B receives 400 μg/kg of Peptide 1 per i.v. infusion.

The tested liquid composition has a pH of 8.2-8.4, and comprises the following:
i) 5 mg/ml of Peptide 1,
ii) 4% (w/w) mannitol,
iii) 1% (w/w) sucrose,
iv) 0.01% (w/w) Polysorbate-80,
v) 10 mM glycyl-glycine, and
vi) sterile water for injection.

In these groups, 6 subjects receives the active treatment (three separate 30-min intravenous (i.v.) infusions of Peptide 1, wherein each i.v. infusion corresponds to the above doses) and 2 receives the placebo. In order to prevent any anticipated unblinding, the subjects under active treatment are recorded by the bioanalytical laboratory.

Blood samples are collected for PK purpose for Peptide 1 determinations on Day 1 at the following time points: pre-dose, then, 10 min, 20 min, 30 min (30 min=0.5), 0.75, 1.0, 1.5, 2.0, 2.5, 3.0, 4.0, 6.0, 8.0, 12.0, 16.0 and 24.0 h after the infusion start.

For the interim analysis, only plasma concentrations of Peptide 1 is considered. The pharmacokinetics analyses are conducted as described under “Pharmacokinetics Analyses” in Example 4.

### Example 6: Clinical Test in Humans—Multiple Doses

A total of 8 subjects were included in each group. In each group, 6 subjects received the active treatment and 2 received the placebo. The active treatment was as follows in each group:
Group 9 received three 8 hours apart 30-min Peptide 1 intravenous (i.v.) infusions, wherein each i.v. infusion corresponded to a dose of 50 μg/kg.
Group 10 received three 8 hours apart 30-min Peptide 1 intravenous (i.v.) infusions, wherein each i.v. infusion corresponded to a dose of 100 μg/kg.
Group 11 received three 8 hours apart 30-min Peptide 1 intravenous (i.v.) infusions, wherein each i.v. infusion corresponded to a dose of 140 μg/kg.
Group 12 received three 8 hours apart 30-min Peptide 1 intravenous (i.v.) infusions, wherein each i.v. infusion corresponded to a dose of 200 μg/kg.
Group 13 received three 8 hours apart 30-min Peptide 1 intravenous (i.v.) infusions, wherein each i.v. infusion corresponded to a dose of 250 μg/kg.

In order to prevent any anticipated unblinding, the subjects under active treatment were recorded by the bioanalytical laboratory.

The tested liquid pharmaceutical composition has a pH of 8.2-8.4, and comprises the following:
i) 5 mg/ml of Peptide 1,
ii) 4% (w/w) mannitol,
iii) 1% (w/w) sucrose,
iv) 0.01% (w/w) Polysorbate-80,
v) 10 mM glycyl-glycine, and
vi) sterile water for injection.

Blood samples were collected for PK purpose for Peptide 1 determinations on Day 1 at the following time points: pre-dose, then 10 min, 20 min, 30 min=0.5, 0.75, 1.0, 1.5, 2.0, 2.5, 3.0, 4.0, 6.0, 8.0, then 10 min, 20 min, 30 min after the second infusion start, i.e. 8.16, 8.32, 8.5, 8.75, 9.0, 9.5, 10.0, 10.5, 11.0, 12.0, 14.0, 16.0, then 10 min, 20 min, 30 min after the second infusion start, i.e. 16.16, 16.32, 16.5, 16.75, 17.0, 17.5, 18.0, 18.5, 19.0, 20.0, 22.0 and 24 hours after the first infusion start.

For the interim analysis, only plasma concentrations of Peptide 1 were considered.

### Pharmacokinetics Analyses

Venous whole blood samples were collected from all subjects.

Plasma samples were analyzed for Peptide 1 using validated Liquid chromatography—Tandem Mass spectrometry (LC-MS/MS) methods by Charles River Laboratories (CRL).

Derivation of PK parameters were carried out by PhinC Development using Phoenix WinNonlin® (Version Phoenix 8.1—Certara—Princeton—USA). A non-compartmental analysis (NCA) approach was chosen.

For the calculation of the PK parameters and characteristics the following rules were applied:
All the plasma concentrations validated by the bioanalytical laboratory and provided to the pharmacokinetics were used for the PK analysis.
The theoretical PK blood sampling time points were used.
At time points in the lag-time between time zero and the first concentration equal or above limit of quantification (LOQ), concentrations below the LOQ (BLQ) were set to zero (0).
Concentrations BLQ between 2 concentrations equal or above LOQ were considered as missing.
Trailing concentrations (at time point between time of last measurable concentrations and time of last blood sample) BLQ were not used in calculations.
For 3 infusions regimen, each infusion was considered separately and BLQ rules were applied for each infusion. For example, if time point 8 h was BLQ, this BLQ value was not used for the determination of PK parameters of the first infusion and was set to zero for the determination of PK parameters of the second infusion.
For concentration above the upper limit of quantification and reported as above limit of quantification (ALQ) in the final plasma concentration tables, ALQ was considered as missing for the PK analysis.
Not reported (NR) concentration was excluded from the PK analysis.
For the first administration, if pre-dose concentration was less than or equal to 5% of Cmax value in a PK profile, the subject's data was included in all PK measurements and calculations without any adjustments. If the pre-dose value was greater than 5% of Cmax, the subject was dropped from all statistical evaluations.

The following PK parameters of Peptide 1, were derived for each subject receiving the active treatment on Day 1:
Cₘₐₓ: The observed maximum concentration in plasma measured in a subject after dosing identified by inspection of the plasma drug concentration versus (vs.) time data by WinNonlin® after each administration (Cₘₐₓ₁, Cₘₐₓ₂, Cₘₐₓ₃).
tₘₐₓ: The time at which Cmax was apparent, identified by inspection of the plasma drug concentration vs. time data by Phoenix WinNonlin® (tₘₐₓ₁, tₘₐₓ₂, tₘₐₓ₃).
Global Cₘₐₓ: The overall observed maximum concentration measured was obtained directly from the concentrate-time data.
Global tₘₐₓ: The time at which global Cₘₐₓ was apparent, identified by inspection of the drug concentration vs. time data by Phoenix WinNonlin®.
AUC₀₋ₜₐᵤ: The area under the concentration-time curve over the dosing interval tau (i.e. 8 h) was calculated using a linear trapezoidal method after each administration (AUC₀₋ₜₐᵤ₁, AUC₀₋ₜₐᵤ₂ and AUC₀₋ₜₐᵤ₃, where AUC₀₋ₜₐᵤ was the AUC₀₋ₜₐᵤ from pre-dose to 8 h, AUC₀₋ₜₐᵤ₂ was the AUC₀₋ₜₐᵤ from 8 h to 16 h and AUC₀₋ₜₐᵤ₃ was the AUC₀₋ₜₐᵤ from 16 h to 24 h). AUC₀₋ₜₐᵤ was only calculated if there were at least 3 concentrations above LOQ.
AUC₀₋₂₄: The area under the plasma concentration-time curve from time zero (pre-dose) to 24 h was calculated as the sum of the 3 AUC₀₋ₜₐᵤ: AUC₀₋₂₄=AUC₀₋ₜₐᵤ₁+AUC₀₋ₜₐᵤ₂+AUC₀₋ₜₐᵤ₃.
AUC₀₋ₜ: The area under the concentration-time curve from time zero (pre-dose) to the time of last quantifiable concentration was calculated using a linear trapezoidal method after each administration (AUC₀₋ₜ₁, AUC₀₋ₜ₂ and AUC₀₋ₜ₃, where AUC₀₋ₜ₁ was AUC₀₋ₜ corresponded to the AUC from pre-dose to the last measurable quantifiable concentration following the first infusion). AUC₀₋ₜ was only calculated if there were at least 3 concentrations above LOQ.
kₑ: The terminal plasma elimination rate-constant was estimated from log-linear regression analysis of the terminal phase of the plasma concentration-time profile. The number of points included in the terminal phase was determined by visual inspection of the semi-log plots of the plasma concentration-time profiles. A minimum of 3 data points, including the last measured data point and excluding Cₘₐₓ, had to be available for the regression. The correlation coefficient for the goodness of the fit of the regression line through the data points (r²) had to be 0.8000 or higher, for the value to be considered reliable. No determination of derived parameters (t_{1/2}) was performed with unreliable elimination rate constants (hence, considered as not calculable-NC). Only after the last infusion.
t_{1/2}: The apparent terminal elimination half-life was calculated as In2/kₑ, where kₑ is the elimination rate constant as defined above. Only after the last infusion.
Racc: Ratio of accumulation on Cₘₐₓ and AUC₀₋ₜₐᵤ between the first and the last administrations.

Descriptive statistics for the concentrations were presented as number of observations (N), arithmetic mean, standard deviation (SD) and were calculated if at least ⅔ of the concentrations per time-point were non-missing. For descriptive statistics calculations, concentration BLQ were set to zero (0) before the first concentration equal or above LOQ and considered as missing after (this rule was applied to each infusion separately).

Descriptive statistics of the PK parameters were presented as N, arithmetic mean, SD, coefficient of variation (CV %), median, geometric mean (GM), minimum (Min) and maximum (Max), and were calculated if N≥3.

### Main Pharmacokinetics Results

Main data and descriptive statistics of subjects in Group 9 (three i.v. infusions of 50 μg/kg), Group 10 (three i.v. infusions of 100 μg/kg), Group 11 (three i.v. infusions of 140 μg/kg), Group 12 (three i.v. infusions of 200 μg/kg) and Group 13 (three i.v. infusions of 250 μg/kg) are shown in Table 5A, 5B and 5C.

TABLE 5A
Summary of main PK parameters by dose level (three 8 hours apart 30-
min Peptide 1 intravenous (i.v.) infusions, each i.v. infusion corresponding
to 50 μg/kg, 100 μg/kg, 140 μg/kg, 200 μg/kg or 250 μg/kg).

| Dose level | | tₘₐₓ₁# | Cₘₐₓ₁ | AUC₀₋ₜ₁ | AUC₀₋ₜₐᵤ₁ | tₘₐₓ₂# | Cₘₐₓ₂ |
|---|---|---|---|---|---|---|---|
| (μg/kg) | | (h) | (ng/mL) | (h*ng/mL) | (h*ng/mL) | (h) | (ng/mL) |
| 50 | N | 6 | 6 | 6 | 6 | 6 | 6 |
| | Mean | 0.50 | 406 | 421.51 | 513.19 | 8.50 | 395 |
| | SD | 0.00 | 49.8 | 66.41 | 67.29 | 0.00 | 61.8 |
| | CV % | 0.50-0.50 | 12.3 | 15.8 | 13.1 | 8.50-8.50 | 15.7 |
| | GM | 0.50 | 403 | 417.03 | 509.22 | 8.50 | 391 |
| 100 | N | 6 | 6 | 6 | 6 | 6 | 6 |
| | Mean | 0.50 | 883 | 992.51 | 1115.91 | 8.50 | 977 |
| | SD | 0.00 | 117 | 160.71 | 183.86 | 0.00 | 121 |
| | CV % | 0.50-0.50 | 13.2 | 16.2 | 16.5 | 8.50-8.50 | 12.4 |
| | GM | 0.50 | 877 | 981.90 | 1103.58 | 8.50 | 971 |
| 140 | N | 6 | 6 | 6 | 6 | 6 | 6 |
| | Mean | 0.50 | 1340 | 1688.47 | 1762.04 | 8.50 | 1390 |
| | SD | 0.00 | 277 | 296.64 | 296.94 | 0.07 | 327 |
| | CV % | 0.50-0.50 | 20.7 | 17.6 | 16.9 | 8.32-8.50 | 23.6 |
| | GM | 0.50 | 1310 | 1664.37 | 1739.74 | 8.47 | 1360 |
| 200 | N | 6 | 6 | 6 | 6 | 6 | 6 |
| | Mean | 0.50 | 2190 | 2519.95 | 2616.99 | 8.50 | 2050 |
| | SD | 0.00 | 370 | 441.23 | 428.66 | 0.00 | 361 |
| | CV % | 0.50-0.50 | 16.9 | 17.5 | 16.4 | 8.50-8.50 | 17.6 |
| | GM | 0.50 | 2170 | 2488.89 | 2588.79 | 8.50 | 2020 |
| 250 | N | 6 | 6 | 6 | 6 | 6 | 6 |
| | Mean | 0.50 | 1960 | 2539.82 | 2628.97 | 8.50 | 1930 |
| | SD | 0.07 | 519 | 502.67 | 468.94 | 0.10 | 878 |
| | CV % | 0.32-0.50 | 26.5 | 19.8 | 17.8 | 8.50-8.75 | 45.4 |
| | GM | 0.46 | 1890 | 2495.61 | 2591.00 | 8.54 | 1800 |

#Median and Min-Max instead of Mean and CV %.

TABLE 5B
Summary of main PK parameters by dose level (three 8 hours apart 30-
min Peptide 1 intravenous (i.v.) infusions, each i.v. infusion corresponding
to 50 μg/kg, 100 μg/kg, 140 μg/kg, 200 μg/kg or 250 μg/kg).

| Dose level | | AUC₀₋ₜ₂ | AUC₀₋ₜₐᵤ₂ | tₘₐₓ₃# | Cₘₐₓ₃ | AUC₀₋ₜ₃ | AUC₀₋ₜₐᵤ₃ |
|---|---|---|---|---|---|---|---|
| (μg/kg) | | (h*ng/mL) | (h*ng/mL) | (h) | (ng/mL) | (h*ng/mL) | (h*ng/mL) |
| 50 | N | 6 | 6 | 6 | 6 | 6 | 6 |
| | Mean | 420.15 | 517.24 | 16.50 | 398 | 436.79 | 541.41 |
| | SD | 66.90 | 88.28 | 0.00 | 49.0 | 59.95 | 69.80 |
| | CV % | 15.9 | 17.1 | 16.50-16.50 | 12.3 | 13.7 | 12.9 |
| | GM | 415.76 | 510.82 | 16.50 | 396 | 433.27 | 537.64 |
| 100 | N | 6 | 6 | 6 | 6 | 6 | 6 |
| | Mean | 1128.25 | 1242.21 | 16.50 | 970 | 1091.00 | 1262.51 |
| | SD | 135.83 | 148.43 | 0.00 | 172 | 121.08 | 143.13 |
| | CV % | 12.0 | 11.9 | 16.50-16.50 | 17.7 | 11.1 | 11.3 |
| | GM | 1121.43 | 1235.35 | 16.50 | 958 | 1085.21 | 1255.07 |
| 140 | N | 6 | 6 | 6 | 6 | 6 | 6 |
| | Mean | 1870.56 | 1943.20 | 16.50 | 1240 | 1596.51 | 1706.06 |
| | SD | 730.20 | 696.75 | 0.00 | 224 | 306.39 | 266.64 |
| | CV % | 39.0 | 35.9 | 16.50-16.50 | 18.0 | 19.2 | 15.6 |
| | GM | 1760.76 | 1845.93 | 16.50 | 1220 | 1570.03 | 1687.67 |
| 200 | N | 6 | 6 | 6 | 6 | 6 | 6 |
| | Mean | 2610.99 | 2690.67 | 16.50 | 2170 | 2723.43 | 2807.44 |
| | SD | 548.54 | 492.21 | 0.07 | 435 | 481.55 | 463.99 |
| | CV % | 21.0 | 18.3 | 16.32-16.50 | 20.1 | 17.7 | 16.5 |
| | GM | 2558.87 | 2651.36 | 16.47 | 2130 | 2687.26 | 2774.47 |
| 250 | N | 6 | 6 | 6 | 6 | 6 | 6 |
| | Mean | 2353.74 | 2440.44 | 16.50 | 1800 | 2431.49 | 2481.57 |
| | SD | 588.43 | 583.67 | 0.09 | 265 | 390.70 | 366.51 |
| | CV % | 25.0 | 23.9 | 16.32-16.50 | 14.7 | 16.1 | 14.8 |
| | GM | 2298.71 | 2387.75 | 16.44 | 1780 | 2405.33 | 2459.24 |

#Median and Min-Max instead of Mean and CV %.

TABLE 5C
Summary of main PK parameters by dose level (three 8 hours apart 30-
min Peptide 1 intravenous (i.v.) infusions, each i.v. infusion corresponding
to 50 μg/kg, 100 μg/kg, 140 μg/kg, 200 μg/kg or 250 μg/kg).

| | | Global | Global | | | | |
|---|---|---|---|---|---|---|---|
| Dose level | | tₘₐₓ# | Cₘₐₓ | AUC₀₋₂₄ | Racc | Racc | t_{1/2} |
| (μg/kg) | | (h) | (ng/mL) | (h*ng/mL) | Cₘₐₓ | AUC₀₋ₜₐᵤ | (h) |
| 50 | N | 6 | 6 | 6 | 6 | 6 | 6 |
| | Mean | 8.50 | 422 | 1571.85 | 0.99 | 1.07 | 1.38 |
| | SD | 7.16 | 44.0 | 195.19 | 0.12 | 0.19 | 0.21 |
| | CV % | 0.50-16.50 | 10.4 | 12.4 | 12.6 | 17.4 | 15.2 |
| | GM | 4.12 | 420 | 1561.84 | 0.98 | 1.06 | 1.37 |
| 100 | N | 6 | 6 | 6 | 6 | 6 | 6 |
| | Mean | 12.50 | 1020 | 3620.63 | 1.10 | 1.15 | 2.18 |
| | SD | 4.38 | 152 | 421.55 | 0.08 | 0.16 | 0.90 |
| | CV % | 8.50-16.50 | 14.8 | 11.6 | 7.3 | 13.8 | 41.4 |
| | GM | 11.84 | 1020 | 3600.82 | 1.09 | 1.14 | 2.04 |
| 140 | N | 6 | 6 | 6 | 6 | 6 | 6 |
| | Mean | 8.41 | 1430 | 5411.31 | 0.93 | 0.97 | 1.94 |
| | SD | 4.11 | 345 | 1112.25 | 0.06 | 0.08 | 0.23 |
| | CV % | 0.50-8.50 | 24.1 | 20.6 | 6.2 | 8.1 | 11.8 |
| | GM | 3.29 | 1400 | 5309.74 | 0.93 | 0.97 | 1.93 |
| 200 | N | 6 | 6 | 6 | 6 | 6 | 6 |
| | Mean | 4.50 | 2370 | 8115.10 | 1.00 | 1.07 | 1.94 |
| | SD | 7.87 | 300 | 1360.88 | 0.22 | 0.07 | 0.23 |
| | CV % | 0.50-16.50 | 12.7 | 16.8 | 22.3 | 6.7 | 11.7 |
| | GM | 2.57 | 2350 | 8018.92 | 0.98 | 1.07 | 1.93 |
| 250 | N | 6 | 6 | 6 | 6 | 5 | 5 |
| | Mean | 8.50 | 2330 | 7550.98 | 0.96 | 0.97 | 1.88 |
| | SD | 6.02 | 662 | 1033.56 | 0.20 | 0.23 | 0.41 |
| | CV % | 0.50-16.50 | 28.4 | 13.7 | 21.1 | 23.3 | 21.7 |
| | GM | 3.69 | 2260 | 7494.93 | 0.94 | 0.95 | 1.85 |

#Median and Min-Max instead of Mean and CV %.

### Conclusion

Following three 8 hours apart 30-min Peptide 1 intravenous (i.v.) infusions corresponding to three times administration of Peptide 1 in dose range 50-250 μg/kg:
For this dose regimen, it should be noted that each infusion was considered separately and BLQ rules were applied for each infusion. For example, if time point 8 h was BLQ, this BLQ value was not used for the determination of PK parameters of the first infusion and was set to zero for the determination of PK parameters of the second infusion
For Group 12 (three 8 hours apart 30-min Peptide 1 intravenous (i.v.) infusions, wherein each i.v. infusion corresponds to a dose of 200 μg/kg), in all subjects and for each infusion, Peptide 1 pre-dose concentrations were BLQ and the first concentrations were measurable 0.16 h after the start of infusion, expect for two subjects for whom the pre-dose concentration of the third administration (i.e. 16 h) was measurable. Concentrations were measurable in all subjects up to at least 4 h after each infusion. It should be noted that for two subjects, concentrations were measurable up to 8 h after the second administration and for one subject, concentrations were measurable up to 8 h after the third administration.
For Group 13 (three 8 hours apart 30-min Peptide 1 intravenous (i.v.) infusions, wherein each i.v. infusion corresponds to a dose of 250 μg/kg), in all subjects and for each infusion, Peptide 1 pre-dose concentrations were BLOQ and the first concentrations were measurable 0.16 h after the start of infusion, expect for two subjects for whom the pre-dose concentration of the second administration (8 h) was measurable, and for one subject whom the pre-dose concentration of the third administration (16 h) was measurable. Concentrations were measurable in all subjects up to at least 4 h after each infusion. It should be noted that for two subjects, concentration at the timepoint 24 h was measurable.
After three 8 hours apart 30-min intravenous infusions, concentrations increased up to Cₘₐₓ with tₘₐₓ observed at 0.5 h (time of the end of infusion) after each administration for all subjects except for one subject for whom Cₘₐₓ was observed at 0.32 h after the third administration. However, concentrations were identical 0.32 h and 0.50 h after the third administration for this subject. Then concentrations decreased.
Exposure parameters (Global Cₘₐₓ, Cₘₐₓ and AUCs) were consistent between subjects and infusions with low inter-individual variability (CV % ranging from 12.7% to 45.4%).
For dose comparison:
   Between 50 μg/kg t.i.d. and 250 μg/kg t.i.d., GM of Global Cₘₐₓ increased by 5.4-fold and GM of AUC₀₋₂₄ increased by 4.8-fold when the dose of Peptide 1 increased by 5-fold,
   between 100 μg/kg t.i.d. and 250 μg/kg t.i.d., GM of Global Cₘₐₓ increased by 2.2-fold and GM of AUC₀₋₂₄ increased by 2.1-fold when the dose of Peptide 1 increased by 2.5-fold,
   between 140 μg/kg t.i.d. and 250 μg/kg t.i.d., GM of Global Cₘₐₓ increased by 1.6-fold and GM of AUC₀₋₂₄ increased by 1.4-fold when the dose of Peptide 1 increased by 1.8-fold,
   between 200 μg/kg t.i.d. and 250 μg/kg t.i.d., GM of Global Cₘₐₓ increased by 1.0-fold and GM of AUC₀₋₂₄ increased by 0.9-fold when the dose of Peptide 1 increased by 1.25-fold.
No accumulation was observed between the first and the last infusion as showed by GM accumulation ratios on Cₘₐₓ and AUC₀₋ₜₐᵤ around 1 with low inter-individual variability (CV % below 24%).
For Group 12 (three 8 hours apart 30-min Peptide 1 intravenous (i.v.) infusions, wherein each i.v. infusion corresponds to a dose of 200 μg/kg), elimination phase could be determined for all subjects after the last infusion, with GM t_{1/2} around 2 h which was consistent with the GM t_{1/2} value observed in previous group after three 8 hours apart 30-min intravenous infusions corresponding to 140 μg/kg (Group 11: t_{1/2} of 1.93 h).
For Group 13 (three 8 hours apart 30-min Peptide 1 intravenous (i.v.) infusions, wherein each i.v. infusion corresponds to a dose of 250 μg/kg), elimination phase could be determined for all subjects after the last infusion, except for one subject for whom r² was below 0.8. GM t_{1/2} value was around 2 h.
At 200 μg/kg t.i.d., GM of AUC₀₋ₜₐᵤ₁, AUC₀₋ₜₐᵤ₂ and AUC₀₋ₜₐᵤ₃ were below the stopping rule (2590 h·ng/ml, 2750 h·ng/ml and 2708 h·ng/ml, respectively, for a stopping rule of 4654 h·ng/ml). It should be noted that in this dose level Peptide 1 was not completely eliminated for all subjects before the next administration.

Sequence overview
Full length Annexin A1 (homo sapiens) has the
following sequence:
>sp|P04083|ANXA1_HUMAN Annexin A1 OS = Homo
sapiens GN = ANXA1 PE = 1 SV = 2
(SEQ ID NO: 1)
MAMVSEFLKQAWFIENEEQEYVQTVKSSKGGPGSAVSPYPTFNPSSDVAAL
HKAIMVKGVDEATIIDILTKRNNAQRQQIKAAYLQETGKPLDETLKKALTG
HLEEVVLALLKTPAQFDADELRAAMKGLGTDEDTLIEILASRTNKEIRDIN
RVYREELKRDLAKDITSDTSGDFRNALLSLAKGDRSEDFGVNEDLADSDAR
ALYEAGERRKGTDVNVFNTILTTRSYPQLRRVFQKYTKYSKHDMNKVLDLE
LKGDIEKCLTAIVKCATSKPAFFAEKLHQAMKGVGTRHKALIRIMVSRSEI
DMNDIKAFYQKMYGISLCQAILDETKGDYEKILVALCGGN.
(SED ID NO: 2; Annexin A1 2-50)
AMVSEFLKQAWFIENEEQEYVQTVKSSKGGPGSAVSPYPTFNPSSDVAA
(SED ID NO: 3; Annexin A1 2-48)
AMVSEFLKQAWFIENEEQEYVQTVKSSKGGPGSAVSPYPTFNPSSDV
(SED ID NO: 4; Annexin A1 2-46)
AMVSEFLKQAWFIENEEQEYVQTVKSSKGGPGSAVSPYPTFNPSS
(SED ID NO: 5; Annexin A1 2-50 V24L)
AMVSEFLKQAWFIENEEQEYVQTLKSSKGGPGSAVSPYPTFNPSSDVAA
(SED ID NO: 6; Annexin A1 2-48 V24L)
AMVSEFLKQAWFIENEEQEYVQTLKSSKGGPGSAVSPYPTFNPSSDV
(SED ID NO: 7; Annexin A1 2-46 V24L)
AMVSEFLKQAWFIENEEQEYVQTLKSSKGGPGSAVSPYPTFNPSS
(SED ID NO: 8; Annexin A1 2-26)
AMVSEFLKQAWFIENEEQEYVQTVK,
(SED ID NO: 9; Annexin A1 2-26 V24L)
AMVSEFLKQAWFIENEEQEYVQTLK
All peptides may be C-terminally amidated (-NH₂).

### Items

1. A liquid pharmaceutical composition comprising an Annexin A1 (AnxA1) N-terminal-peptide and at least one pharmaceutically acceptable excipient, wherein said liquid pharmaceutical composition is to be administered to a human subject as a 1-120 minutes intravenous (i.v.) infusion, and wherein said AnxA1 N-terminal-peptide has a terminal elimination half-life (t_{1/2}) of at least 30 minutes after said i.v. infusion.
2. A liquid pharmaceutical composition comprising an Annexin A1 (AnxA1) N-terminal-peptide and at least one pharmaceutically acceptable excipient for use in the treatment of an ischemic condition and/or an inflammatory condition, wherein said liquid pharmaceutical composition is administered to a human subject as a 1-120 minutes intravenous (i.v.) infusion, and wherein said AnxA1 N-terminal-peptide has a terminal elimination half-life (t_{1/2}) of at least 30 minutes after said i.v. infusion.
3. The liquid pharmaceutical composition or liquid pharmaceutical composition for use according to any of the preceding items, wherein said liquid pharmaceutical composition is to be administered as a 10-90 minutes i.v. infusion, such as a 10-60 minutes i.v. infusion, such as a 20-40 minutes i.v. infusion, such as a 30 minutes i.v. infusion.
4. The liquid pharmaceutical composition or liquid pharmaceutical composition for use according to any of the preceding items, wherein said liquid pharmaceutical composition is to be administered as a 30 minutes i.v. infusion.
5. The liquid pharmaceutical composition or liquid pharmaceutical composition for use according to any of the preceding items, wherein said AnxA1 N-terminal-peptide has a t_{1/2} of at least 35 minutes, such as of at least 40 minutes, such as of at least 45 minutes, such as of at least 50 minutes, such as of at least 55 minutes, such as of at least 60 minutes after said i.v. infusion.
6. The liquid pharmaceutical composition or liquid pharmaceutical composition for use according to any of the preceding items, wherein said AnxA1 N-terminal-peptide has a t_{1/2} of at least 35 minutes, such as of at least 45 minutes, such as of at least 60 minutes, such as of at least 75 minutes, such as of at least 90 minutes, such as of at least 120 minutes, such as of at least 180 minutes after said i.v. infusion.
7. The liquid pharmaceutical composition or liquid pharmaceutical composition for use according to any of the preceding items, wherein the i.v. infusion corresponds to administration of a dosage of about 15 μg/kg, about 25 μg/kg, about 50 μg/kg, about 100 μg/kg, about 150 μg/kg, about 200 μg/kg or about 400 μg/kg of said AnxA1 N-terminal-peptide.
8. The liquid pharmaceutical composition or liquid pharmaceutical composition for use according to any of the preceding items, wherein said AnxA1 N-terminal-peptide is selected from the group consisting of:
   a. AMVSEFLKQAWFIENEEQEYVQTVKSSKGGPGSAVSPYPTFNPSSDVAA (SEQ ID NO: 2; Annexin A1 2-50),
   b. AMVSEFLKQAWFIENEEQEYVQTVKSSKGGPGSAVSPYPTFNPSSDV (SEQ ID NO: 3; Annexin A1 2-48),
   c. AMVSEFLKQAWFIENEEQEYVQTVKSSKGGPGSAVSPYPTFNPSS (SEQ ID NO: 4; Annexin A1 2-46),
   d. AMVSEFLKQAWFIENEEQEYVQTLKSSKGGPGSAVSPYPTFNPSSDVAA (SEQ ID NO: 5; Annexin A1 2-50 V24L),
   e. AMVSEFLKQAWFIENEEQEYVQTLKSSKGGPGSAVSPYPTFNPSSDV (SEQ ID NO: 6; Annexin A1 2-48 V24L),
   f. AMVSEFLKQAWFIENEEQEYVQTLKSSKGGPGSAVSPYPTFNPSS (SEQ ID NO: 7; Annexin A1 2-46 V24L), and
   g. a functional variant of any one of SEQ ID NOs: 2-7 comprising 1 to 6 individual amino acid substitutions.
9. The liquid pharmaceutical composition or liquid pharmaceutical composition for use according to item 8, wherein said functional variant comprises 1-3 individual amino acid substitutions, such as 1 individual amino acid substitution, 2 individual amino acid substitutions or 3 individual amino acid substitutions.
10. The liquid pharmaceutical composition or liquid pharmaceutical composition for use according to any of items 8-9, wherein said one or more individual amino acid substitutions are conservative amino acid substitutions.
11. The liquid pharmaceutical composition or liquid pharmaceutical composition for use according to any of items 8-10, wherein said functional variant is a ligand and/or agonist of one or more of Formyl Peptide Receptor 1 (FPR1), Formyl Peptide Receptor 2 (FPR2) and Formyl Peptide Receptor 3 (FPR3).
12. The liquid pharmaceutical composition or liquid pharmaceutical composition for use according to any of items 8-11, wherein said functional variant activates and/or stimulates one or more of Formyl Peptide Receptor 1 (FPR1), Formyl Peptide Receptor 2 (FPR2) and Formyl Peptide Receptor 3 (FPR3).
13. The liquid pharmaceutical composition or liquid pharmaceutical composition for use according to any of the preceding items, wherein said AnxA1 N-terminal-peptide has a C-terminal amidation.
14. The liquid pharmaceutical composition or liquid pharmaceutical composition for use according to any of the preceding items, wherein said AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), optionally with a C-terminal amidation.
15. The liquid pharmaceutical composition or liquid pharmaceutical composition for use according to any of the preceding items, wherein said liquid pharmaceutical composition comprises 0.5-10 mg/mL of said AnxA1 N-terminal-peptide, such as 1-10 mg/mL, such as 2-9 mg/mL, such as 3-8 mg/mL, such as 4-6 mg/mL.
16. The liquid pharmaceutical composition or liquid pharmaceutical composition for use according to any of the preceding items, wherein said liquid pharmaceutical composition comprises about 5 mg/mL of said AnxA1 N-terminal-peptide.

17. The liquid pharmaceutical composition or liquid pharmaceutical composition for use according to any of the preceding items, wherein said liquid pharmaceutical composition has pH>6.0.
18. The liquid pharmaceutical composition or liquid pharmaceutical composition for use according to any of the preceding items, wherein said liquid pharmaceutical composition has pH≥7.4, such as pH≥7.5, such as pH≥7.6, such as pH≥7.7, such as pH≥7.8, such as pH≥7.9, such as pH≥8.0.
19. The liquid pharmaceutical composition or liquid pharmaceutical composition for use according to any of the preceding items, wherein said liquid pharmaceutical composition has pH>8.0.
20. The liquid pharmaceutical composition or liquid pharmaceutical composition for use according to any of the preceding items, wherein said liquid pharmaceutical composition has a pH of about 8.2-8.4, such as of about 8.3.
21. The liquid pharmaceutical composition or liquid pharmaceutical composition for use according to any of the preceding items, said liquid pharmaceutical composition comprising:
   i) one or more sugar alcohols, and/or
   ii) one or more carbohydrates.
22. The liquid pharmaceutical composition or liquid pharmaceutical composition for use according to any of the preceding items, said liquid pharmaceutical composition comprising:
   i) a sugar alcohol selected from the group consisting of mannitol, sorbitol, inositol, glycerol, xylitol, propylene glycol, polyethylene glycols (PEGs) and polypropylene/ethylene glycol copolymer, and/or
   ii) a carbohydrate selected from the group consisting of sucrose, trehalose, mannose, ribose, maltose, glucose, lactose, galactose and arabinose.
23. The liquid pharmaceutical composition or liquid pharmaceutical composition for use according to any of the preceding items, said liquid pharmaceutical composition comprising:
   i) 1-10 mg/mL of said AnxA1 N-terminal-peptide,
   ii) 3-5% (w/w) of a sugar alcohol, and
   iii) 0.5-1.5% (w/w) of a carbohydrate.
24. The liquid pharmaceutical composition or liquid pharmaceutical composition for use according to any of the preceding items, said liquid pharmaceutical composition comprising:
   i) 1-10 mg/ml of said AnxA1 N-terminal-peptide,
   ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer, and
   iii) 0.5-1.5% (w/w) of a carbohydrate selected from the group consisting of sucrose, trehalose, mannose, ribose, maltose, glucose, lactose, galactose and arabinose.
25. The liquid pharmaceutical composition or liquid pharmaceutical composition for use according to any of the preceding items, said liquid pharmaceutical composition comprising:
   i) 1-10 mg/mL of said AnxA1 N-terminal-peptide,
   ii) 3-5% (w/w) of a sugar alcohol,
   iii) 0.5-1.5% (w/w) of a carbohydrate,
   iv) 0.005-0.015% (w/w) of a surfactant, and
   v) 5-15 mM buffer.
26. The liquid pharmaceutical composition or liquid pharmaceutical composition for use according to any of the preceding items, wherein said liquid pharmaceutical composition has a pH of 8.2-8.4, said liquid pharmaceutical composition comprising
   i) 1-10 mg/ml of said AnxA1 N-terminal-peptide,
   ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer,
   iii) 0.5-1.5% (w/w) of a carbohydrate selected from the group consisting of sucrose, trehalose, mannose, ribose, maltose, glucose, lactose, galactose and arabinose,
   iv) 0.005-0.015% (w/w) of a surfactant selected from the group consisting of Polysorbate-80 and Polysorbate-20, and
   v) 5-15 mM buffer selected from the group consisting of Acetate buffer, TRIS, Amine-containing buffers, Amino-acid based buffers such as lysine, hydroxy-lysine histidine and glycylglycine, non-phosphate buffers, bicarbonate buffers, HEPES and borate buffers.
27. The liquid pharmaceutical composition or liquid pharmaceutical composition for use according to any of the preceding items, wherein said liquid pharmaceutical composition comprises sterile water for injection.
28. The liquid pharmaceutical composition or liquid pharmaceutical composition for use according to any of the preceding items, wherein said liquid pharmaceutical composition has a pH of 8.2-8.4, said liquid pharmaceutical composition comprising
   i) 5 mg/mL of a peptide having the sequence AMVSEFLKQAWFIENEEQEYVQTLKSSKGGPGSAVSPYPTFNPSSDV (SEQ ID NO: 6; Annexin A1 2-48 V24L), optionally with a C-terminal amidation,
   ii) 4% (w/w) mannitol,
   iii) 1% (w/w) sucrose,
   iv) 0.01% (w/w) Polysorbate-80,
   v) 10 mM glycyl-glycine, and
   vi) sterile water for injection.
29. The liquid pharmaceutical composition or liquid pharmaceutical composition for use according to any of the preceding items, wherein said liquid pharmaceutical composition is to be administered as a once daily i.v. infusion, such as a once daily 30 minutes i.v. infusion.
30. The liquid pharmaceutical composition or liquid pharmaceutical composition for use according to any of the preceding items, wherein said liquid pharmaceutical composition is to be administered as multiple daily i.v. infusions, such as 2, 3, 4 or 5 daily i.v. infusions.
31. The liquid pharmaceutical composition or liquid pharmaceutical composition for use according to any of the preceding items, wherein said liquid pharmaceutical composition is to be administered as three daily i.v. infusions.
32. The liquid pharmaceutical composition or liquid pharmaceutical composition for use according to any of the preceding items, wherein said liquid pharmaceutical composition is to be administered as three daily i.v. infusions with a dosing interval of 4 to 12 hours, such as a dosing interval of 5 to 11 hours, such as a dosing interval of 6 to 10 hours, such as a dosing interval of 7 to 9 hours.
33. The liquid pharmaceutical composition or liquid pharmaceutical composition for use according to any of the preceding items, wherein said liquid pharmaceutical composition is to be administered as three daily i.v. infusions with a dosing interval of 6 to 10 hours, such as a dosing interval of 6 hours and 30 minutes to 9 hours and 30 minutes, such as 7 to 9 hours, such as a dosing interval of 7 hours and 15 minutes to 8 hours and 45 minutes, such as a dosing interval of 7 hours and 30 minutes to 8 hours and 30 minutes, such as a dosing interval of 7 hours and 45 minutes to 8 hours and 15 minutes.
34. The liquid pharmaceutical composition or liquid pharmaceutical composition for use according to any of the preceding items, wherein said liquid pharmaceutical composition is to be administered as three daily i.v. infusions with a dosing interval of about 8 hours.
35. The liquid pharmaceutical composition or liquid pharmaceutical composition for use according to any of the preceding items, wherein the once daily i.v. infusion or each of the multiple daily i.v. infusions correspond to administration of a dosage of about 15 μg/kg, about 25 μg/kg, about 50 μg/kg, about 100 μg/kg, about 150 μg/kg, about 200 μg/kg, about 250 μg/kg, about 300 μg/kg, about 350 μg/kg or about 400 μg/kg of said AnxA1 N-terminal-peptide.
36. The liquid pharmaceutical composition or liquid pharmaceutical composition for use according to any of the preceding items, wherein the once daily i.v. infusion or each of the multiple daily i.v. infusions correspond to administration of a dosage of about 15-400 μg/kg, about 25-400 μg/kg, about 50-400 μg/kg, about 100-400 μg/kg, about 150-400 μg/kg, or about 200-400 μg/kg of said AnxA1 N-terminal-peptide.
37. The liquid pharmaceutical composition or liquid pharmaceutical composition for use according to any of the preceding items, wherein the once daily i.v. infusion or each of the multiple daily i.v. infusions correspond to administration of a dosage of about 15-400 μg/kg, about 25-400 μg/kg, about 25-200 μg/kg, about 25-150 μg/kg, about 25-100 μg/kg, or about 25-50 μg/kg of said AnxA1 N-terminal-peptide.
38. The liquid pharmaceutical composition or liquid pharmaceutical composition for use according to any of the preceding items, wherein the once daily i.v. infusion or each of the multiple daily i.v. infusions correspond to administration of a dosage of about 25-200 μg/kg of said AnxA1 N-terminal-peptide.
39. The liquid pharmaceutical composition or liquid pharmaceutical composition for use according to any of the preceding items, wherein the once daily i.v. infusion or each of the multiple daily i.v. infusions correspond to administration of a dosage of about 25-250 μg/kg, about 50-250 μg/kg, about 75-250 μg/kg, about 100-250 μg/kg, about 125-250 μg/kg, or about 150-250 μg/kg.
40. The liquid pharmaceutical composition or liquid pharmaceutical composition for use according to any of the preceding items, wherein the once daily i.v. infusion or each of the multiple daily i.v. infusions correspond to administration of a dosage of about 25-200 μg/kg, about 50-200 μg/kg, about 75-200 μg/kg, about 100-200 μg/kg, about 125-200 μg/kg, or about 150-200 μg/kg.
41. The liquid pharmaceutical composition or liquid pharmaceutical composition for use according to any of the preceding items, wherein the once daily i.v. infusion or each of the multiple daily i.v. infusions correspond to administration of a dosage of about 25-200 μg/kg, about 25-175 μg/kg, about 25-150 μg/kg, about 25-125 μg/kg, about 25-100 μg/kg, or about 25-75 μg/kg.
42. The liquid pharmaceutical composition or liquid pharmaceutical composition for use according to any of the preceding items, wherein the once daily i.v. infusion or multiple daily i.v. infusions correspond to administration of a total dosage of about 200-800 μg/kg, about 200-750 μg/kg, about 200-700 μg/kg, about 200-650 μg/kg, about 200-600 μg/kg, about 200-550 μg/kg, about 200-500 μg/kg, about 200-450 μg/kg, or about 200-400 μg/kg.
43. The liquid pharmaceutical composition or liquid pharmaceutical composition for use according to any of the preceding items, wherein the once daily i.v. infusion or multiple daily i.v. infusions correspond to administration of a total dosage of about 200-800 μg/kg, about 250-800 μg/kg, about 300-800 μg/kg, about 350-800 μg/kg, about 400-800 μg/kg, about 450-800 μg/kg, about 500-800 μg/kg, about 550-800 μg/kg, or about 600-800 μg/kg.
44. The liquid pharmaceutical composition or liquid pharmaceutical composition for use according to any of the preceding items, wherein the once daily i.v. infusion or multiple daily i.v. infusions correspond to administration of a total dosage of about 150-350 μg/kg, about 160-340 μg/kg, about 170-330 μg/kg, about 180-320 μg/kg, about 190-310 μg/kg, about 200-300 μg/kg, about 210-290 μg/kg, about 220-280 μg/kg, about 230-270 μg/kg, or about 240-260 μg/kg.
45. The liquid pharmaceutical composition or liquid pharmaceutical composition for use according to any of the preceding items, wherein said liquid pharmaceutical composition is to be administered as 3 daily i.v. infusions.
46. The liquid pharmaceutical composition or liquid pharmaceutical composition for use according to item 45, wherein said liquid pharmaceutical composition is to be administered 3 times daily each as a 20-40 minutes i.v. infusion, such as each as a 30 minutes i.v. infusion.
47. The liquid pharmaceutical composition or liquid pharmaceutical composition for use according to item 45-46, wherein each i.v. infusion corresponds to administration of a dosage of about 25-200 μg/kg, about 25-150 μg/kg, about 25-100 μg/kg, or about 25-50 μg/kg of said AnxA1 N-terminal-peptide.
48. The liquid pharmaceutical composition or liquid pharmaceutical composition for use according to item 45-47, wherein each i.v. infusion corresponds to administration of a dosage of about 50 g/kg of said AnxA1 N-terminal-peptide.
49. The liquid pharmaceutical composition or liquid pharmaceutical composition for use according to any one of the preceding items, wherein each i.v. infusion corresponds to administration of a dosage of 50-250 μg/kg, such as 50-200 μg/kg, such as 50-150 μg/kg, such as 50-140 μg/kg, such as 60-120 μg/kg.
50. The liquid pharmaceutical composition or liquid pharmaceutical composition for use according to item 45-49, wherein said i.v. infusions are administered at time 0 hours, at 8 hours and at 16 hours.
51. The liquid pharmaceutical composition or liquid pharmaceutical composition for use according to any of the preceding items, wherein the mean AUC_{0-24 hrs} of the liquid pharmaceutical composition is selected from the group consisting of:
   i) within about 80.00% to about 125.00% of an AUC_{0-24 hrs} of 211 (CV 2.6%) h·ng/ml, after administration of a single dose of 25 μg/kg,
   ii) within about 80.00% to about 125.00% of an AUC_{0-24 hrs} of 464 (CV 18.8%) h·ng/ml, after administration of a single dose of 50 μg/kg,
   iii) within about 80.00% to about 125.00% of an AUC_{0-24 hrs} of 1108 (CV 13.4%) h·ng/ml, after administration of a single dose of 100 μg/kg,
   iv) within about 80.00% to about 125.00% of an AUC_{0-24 hrs} of 1521 (CV 22.6%) h·ng/ml, after administration of a single dose of 150 μg/kg,
   v) within about 80.00% to about 125.00% of an AUC_{0-24 hrs} of 2278 (CV 3.1%) h·ng/mL, after administration of a single dose of 200 μg/kg, and
   vi) within about 80.00% to about 125.00% of an AUC_{0-24 hrs} of 4872 (CV 11.4%) h·ng/mL, after administration of a single dose of 400 μg/kg.
52. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 211 (CV 2.6%) h·ng/ml, after administration of a single dose of 25 μg/kg.
53. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 464 (CV 18.8%) h·ng/mL, after administration of a single dose of 50 μg/kg.
54. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 1108 (CV 13.4%) h·ng/ml, after administration of a single dose of 100 μg/kg.
55. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 1521 (CV 22.6%) h·ng/ml, after administration of a single dose of 150 μg/kg.
56. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 2278 (CV 3.1%) h·ng/ml, after administration of a single dose of 200 μg/kg.
57. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 4872 (CV 11.4%) h·ng/ml, after administration of a single dose of 400 μg/kg.
58. The liquid pharmaceutical composition or liquid pharmaceutical composition for use according to any of the preceding items, wherein the geometric mean AUC_{0-24 hrs} of the liquid pharmaceutical composition is selected from the group consisting of:
   i) within about 80.00% to about 125.00% of an AUC_{0-24 hrs} of 211 (CV 2.6%) h·ng/ml, after administration of a single dose of 25 μg/kg,
   ii) within about 80.00% to about 125.00% of an AUC_{0-24 hrs} of 457 (CV 18.8%) h·ng/mL, after administration of a single dose of 50 μg/kg,
   iii) within about 80.00% to about 125.00% of an AUC_{0-24 hrs} of 1099 (CV 13.4%) h·ng/ml, after administration of a single dose of 100 μg/kg,
   iv) within about 80.00% to about 125.00% of an AUC_{0-24 hrs} of 1489 (CV 22.6%) h·ng/mL, after administration of a single dose of 150 μg/kg,
   v) within about 80.00% to about 125.00% of an AUC_{0-24 hrs} of 2278 (CV 3.1%) h·ng/ml, after administration of a single dose of 200 μg/kg, and
   vi) within about 80.00% to about 125.00% of an AUC_{0-24 hrs} of 4844 (CV 11.4%) h·ng/ml, after administration of a single dose of 400 μg/kg.
59. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 211 (CV 2.6%) h·ng/ml, after administration of a single dose of 25 μg/kg.
60. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 457 (CV 18.8%) h·ng/ml, after administration of a single dose of 50 μg/kg.
61. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 1099 (CV 13.4%) h·ng/ml, after administration of a single dose of 100 μg/kg.
62. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 1489 (CV 22.6%) h·ng/ml, after administration of a single dose of 150 μg/kg.
63. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 2278 (CV 3.1%) h·ng/ml, after administration of a single dose of 200 μg/kg.
64. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 4844 (CV 11.4%) h·ng/ml, after administration of a single dose of 400 μg/kg.
65. The liquid pharmaceutical composition or liquid pharmaceutical composition for use according to any of the preceding items, wherein the mean AUC₀₋ₜ of the liquid pharmaceutical composition is selected from the group consisting of:
   i) within about 80.00% to about 125.00% of an AUC₀₋ₜ of 133 (CV 19.4%) h·ng/ml, after administration of a single dose of 25 μg/kg,
   ii) within about 80.00% to about 125.00% of an AUC₀₋ₜ of 378 (CV 22.4%) h·ng/ml, after administration of a single dose of 50 μg/kg,
   iii) within about 80.00% to about 125.00% of an AUC₀₋ₜ of 1001 (CV 15.2%) h·ng/ml, after administration of a single dose of 100 μg/kg,
   iv) within about 80.00% to about 125.00% of an AUC₀₋ₜ of 1399 (CV 22.0%) h·ng/ml, after administration of a single dose of 150 μg/kg,
   v) within about 80.00% to about 125.00% of an AUC₀₋ₜ of 2119 (CV 5.2%) h·ng/ml, after administration of a single dose of 200 μg/kg, and
   vi) within about 80.00% to about 125.00% of an AUC₀₋ₜ of 4719 (CV 11.8%) h·ng/ml, after administration of a single dose of 400 μg/kg.
66. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 133 (CV 19.4%) h·ng/ml, after administration of a single dose of 25 μg/kg.
67. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 378 (CV 22.4%) h·ng/ml, after administration of a single dose of 50 μg/kg.
68. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 1001 (CV 15.2%) h·ng/ml, after administration of a single dose of 100 μg/kg.
69. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 1399 (CV 22.0%) h·ng/ml, after administration of a single dose of 150 μg/kg.
70. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 2119 (CV 5.2%) h·ng/ml, after administration of a single dose of 200 μg/kg.
71. The liquid pharmaceutical composition or liquid pharmaceutical composition for use according to any of the preceding items, wherein the mean AUC₀₋ₜ of the liquid pharmaceutical composition is within about 80.00% to about 125.00% of an AUC₀₋ₜ of 4719 (CV 11.8%) h·ng/ml, after administration of a single dose of 400 μg/kg.
72. The liquid pharmaceutical composition or liquid pharmaceutical composition for use according to any of the preceding items, wherein the geometric mean AUC₀₋ₜ of the liquid pharmaceutical composition is selected from the group consisting of:
   i) within about 80.00% to about 125.00% of an AUC₀₋ₜ of 130 (CV 19.4%) h·ng/ml, after administration of a single dose of 25 μg/kg,
   ii) within about 80.00% to about 125.00% of an AUC₀₋ₜ of 369 (CV 22.4%) h·ng/ml, after administration of a single dose of 50 μg/kg,
   iii) within about 80.00% to about 125.00% of an AUC₀₋ₜ of 991 (CV 15.2%) h·ng/ml, after administration of a single dose of 100 μg/kg,
   iv) within about 80.00% to about 125.00% of an AUC₀₋ₜ of 1371 (CV 22.0%) h·ng/ml, after administration of a single dose of 150 μg/kg,
   v) within about 80.00% to about 125.00% of an AUC₀₋ₜ of 2116 (CV 5.2%) h·ng/ml, after administration of a single dose of 200 μg/kg, and
   vi) within about 80.00% to about 125.00% of an AUC₀₋ₜ of 4689 (CV 11.8%) h·ng/ml, after administration of a single dose of 400 μg/kg.
73. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 130 (CV 19.4%) h·ng/ml, after administration of a single dose of 25 μg/kg.
74. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 369 (CV 22.4%) h·ng/ml, after administration of a single dose of 50 μg/kg.
75. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 991 (CV 15.2%) h·ng/ml, after administration of a single dose of 100 μg/kg.
76. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 1371 (CV 22.0%) h·ng/mL, after administration of a single dose of 150 μg/kg.
77. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 2116 (CV 5.2%) h·ng/ml, after administration of a single dose of 200 μg/kg.
78. The liquid pharmaceutical composition or liquid pharmaceutical composition for use according to any of the preceding items, wherein the geometric mean AUC₀₋ₜ of the liquid pharmaceutical composition is within about 80.00% to about 125.00% of an AUC₀₋ₜ of 4689 (CV 11.8%) h·ng/ml, after administration of a single dose of 400 μg/kg.
79. The liquid pharmaceutical composition or liquid pharmaceutical composition for use according to any of the preceding items, wherein the mean AUC_{0-infinity} of the liquid pharmaceutical composition is selected from the group consisting of:
   i) within about 80.00% to about 125.00% of an AUC_{0-infinity} of 211 (CV 19.4%) h·ng/ml, after administration of a single dose of 25 μg/kg,
   ii) within about 80.00% to about 125.00% of an AUC_{0-infinity} of 464 (CV 22.4%) h·ng/ml, after administration of a single dose of 50 μg/kg,
   iii) within about 80.00% to about 125.00% of an AUC_{0-infinity} of 1108 (CV 15.2%) h·ng/ml, after administration of a single dose of 100 μg/kg,
   iv) within about 80.00% to about 125.00% of an AUC_{0-infinity} of 1521 (CV 22.0%) h·ng/ml, after administration of a single dose of 150 μg/kg,
   v) within about 80.00% to about 125.00% of an AUC_{0-infinity} of 2278 (CV 5.2%) h·ng/ml, after administration of a single dose of 200 μg/kg, and
   vi) within about 80.00% to about 125.00% of an AUC_{0-infinity} of 4873 (CV 11.8%) h·ng/mL, after administration of a single dose of 400 μg/kg.
80. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 211 (CV 19.4%) h·ng/ml, after administration of a single dose of 25 μg/kg.
81. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 464 (CV 22.4%) h·ng/ml, after administration of a single dose of 50 μg/kg.
82. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 1108 (CV 15.2%) h·ng/ml, after administration of a single dose of 100 μg/kg.
83. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 1521 (CV 22.0%) h·ng/ml, after administration of a single dose of 150 μg/kg.
84. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 2278 (CV 5.2%) h·ng/ml, after administration of a single dose of 200 μg/kg.
85. The liquid pharmaceutical composition or liquid pharmaceutical composition for use according to any of the preceding items, wherein the mean AUC_{0-infinity} of the liquid pharmaceutical composition is within about 80.00% to about 125.00% of an AUC_{0-infinity} of 4873 (CV 11.8%) h·ng/ml, after administration of a single dose of 400 μg/kg.
86. The liquid pharmaceutical composition or liquid pharmaceutical composition for use according to any of the preceding items, wherein the geometric mean AUC_{0-infinity} of the liquid pharmaceutical composition is selected from the group consisting of:
   i) within about 80.00% to about 125.00% of an AUC_{0-infinity} of 211 (CV 19.4%) h·ng/ml, after administration of a single dose of 25 μg/kg,
   ii) within about 80.00% to about 125.00% of an AUC_{0-infinity} of 456 (CV 22.4%) h·ng/ml, after administration of a single dose of 50 μg/kg,
   iii) within about 80.00% to about 125.00% of an AUC_{0-infinity} of 1099 (CV 15.2%) h·ng/ml, after administration of a single dose of 100 μg/kg,
   iv) within about 80.00% to about 125.00% of an AUC_{0-infinity} of 1490 (CV 22.0%) h·ng/ml, after administration of a single dose of 150 μg/kg,
   v) within about 80.00% to about 125.00% of an AUC_{0-infinity} of 2277 (CV 5.2%) h·ng/ml, after administration of a single dose of 200 μg/kg, and
   vi) within about 80.00% to about 125.00% of an AUC_{0-infinity} of 4845 (CV 11.8%) h·ng/mL, after administration of a single dose of 400 μg/kg.
87. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 211 (CV 19.4%) h·ng/ml, after administration of a single dose of 25 μg/kg.
88. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 456 (CV 22.4%) h·ng/ml, after administration of a single dose of 50 μg/kg.
89. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 1099 (CV 15.2%) h·ng/mL, after administration of a single dose of 100 μg/kg.
90. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 1490 (CV 22.0%) h·ng/ml, after administration of a single dose of 150 μg/kg.
91. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 2277 (CV 5.2%) h·ng/mL, after administration of a single dose of 200 μg/kg.
92. The liquid pharmaceutical composition or liquid pharmaceutical composition for use according to any of the preceding items, wherein the geometric mean AUC_{0-infinity} of the liquid pharmaceutical composition is within about 80.00% to about 125.00% of an AUC_{0-infinity} of 4845 (CV 11.8%) h·ng/ml, after administration of a single dose of 400 μg/kg.
93. The liquid pharmaceutical composition or liquid pharmaceutical composition for use according to any of the preceding items, wherein the mean Cₘₐₓ of the liquid pharmaceutical composition is selected from the group consisting of:
   i) within about 80.00% to about 125.00% of a Cₘₐₓ of 162 (CV 11.7%) ng/ml, after administration of a single dose of 25 μg/kg,
   ii) within about 80.00% to about 125.00% of a Cₘₐₓ of 375 (CV 15.3%) ng/ml, after administration of a single dose of 50 μg/kg,
   iii) within about 80.00% to about 125.00% of a Cₘₐₓ of 916 (CV 21.0%) ng/ml, after administration of a single dose of 100 μg/kg,
   iv) within about 80.00% to about 125.00% of a Cₘₐₓ of 1280 (CV 23.3%) ng/ml, after administration of a single dose of 150 μg/kg,
   v) within about 80.00% to about 125.00% of a Cₘₐₓ of 1700 (CV 4.4%) ng/ml, after administration of a single dose of 200 μg/kg, and
   vi) within about 80.00% to about 125.00% of a Cₘₐₓ of 3420 (CV 10.7%) ng/ml, after administration of a single dose of 400 μg/kg.
94. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 162 (CV 11.7%) ng/ml, after administration of a single dose of 25 μg/kg.
95. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 375 (CV 15.3%) ng/ml, after administration of a single dose of 50 μg/kg.
96. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 916 (CV 21.0%) ng/ml, after administration of a single dose of 100 μg/kg.
97. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 1280 (CV 23.3%) ng/ml, after administration of a single dose of 150 μg/kg.
98. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 1700 (CV 4.4%) ng/ml, after administration of a single dose of 200 μg/kg.
99. The liquid pharmaceutical composition or liquid pharmaceutical composition for use according to any of the preceding items, wherein the mean Cₘₐₓ of the liquid pharmaceutical composition is within about 80.00% to about 125.00% of a Cₘₐₓ of 3420 (CV 10.7%) ng/ml, after administration of a single dose of 400 μg/kg.
100. The liquid pharmaceutical composition or liquid pharmaceutical composition for use according to any of the preceding items, wherein the geometric mean Cₘₐₓ of the liquid pharmaceutical composition is selected from the group consisting of:
   i) within about 80.00% to about 125.00% of a Cₘₐₓ of 161 (CV 11.7%) ng/ml, after administration of a single dose of 25 μg/kg,
   ii) within about 80.00% to about 125.00% of a Cₘₐₓ of 371 (CV 15.3%) ng/ml, after administration of a single dose of 50 μg/kg,
   iii) within about 80.00% to about 125.00% of a Cₘₐₓ of 899 (CV 21.0%) ng/ml, after administration of a single dose of 100 μg/kg,
   iv) within about 80.00% to about 125.00% of a Cₘₐₓ of 1250 (CV 23.3%) ng/ml, after administration of a single dose of 150 μg/kg,
   v) within about 80.00% to about 125.00% of a Cₘₐₓ of 1700 (CV 4.4%) ng/ml, after administration of a single dose of 200 μg/kg, and
   vi) within about 80.00% to about 125.00% of a Cₘₐₓ of 3400 (CV 10.7%) ng/mL, after administration of a single dose of 400 μg/kg.
101. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 161 (CV 11.7%) ng/ml, after administration of a single dose of 25 μg/kg.
102. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 371 (CV 15.3%) ng/ml, after administration of a single dose of 50 μg/kg.
103. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 899 (CV 21.0%) ng/mL, after administration of a single dose of 100 μg/kg.
104. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 1250 (CV 23.3%) ng/ml, after administration of a single dose of 150 μg/kg.
105. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 1700 (CV 4.4%) ng/mL, after administration of a single dose of 200 g/kg.
106. The liquid pharmaceutical composition or liquid pharmaceutical composition for use according to any of the preceding items, wherein the geometric mean Cₘₐₓ of the liquid pharmaceutical composition is within about 80.00% to about 125.00% of a Cₘₐₓ of 3400 (CV 10.7%) ng/ml, after administration of a single dose of 400 μg/kg.
107. The liquid pharmaceutical composition or liquid pharmaceutical composition for use according to any of items 51-106, wherein said single dose is administered as a 30-minute i.v. infusion to a human subject.
108. The liquid pharmaceutical composition or liquid pharmaceutical composition for use according to any of items 51-107, wherein said AUC_{0-24 hrs}, AUC₀₋ₜ, AUC_{0-24 infinity} or Cₘₐₓ is measured after administration of a single dose to a human subject, such as a healthy male Caucasian subject.
109. The liquid pharmaceutical composition or liquid pharmaceutical composition for use according to any of the preceding items, wherein the mean AUC_{0-24 hrs} of the liquid pharmaceutical composition is selected from the group consisting of:
   a. within about 80.00% to about 125.00% of an AUC_{0-24 hrs} of 1572 (CV 12.4%) h·ng/ml, after administration of three doses with a dosing interval of 8 hours, wherein each of said three doses corresponds to 50 μg/kg,
   b. within about 80.00% to about 125.00% of an AUC_{0-24 hrs} of 3621 (CV 11.6%) h·ng/mL, after administration of three doses with a dosing interval of 8 hours, wherein each of said three doses corresponds to 100 μg/kg,
   c. within about 80.00% to about 125.00% of an AUC_{0-24 hrs} of 5411 (CV 20.6%) h·ng/ml, after administration of three doses with a dosing interval of 8 hours, wherein each of said three doses corresponds to 140 μg/kg,
   d. within about 80.00% to about 125.00% of an AUC_{0-24 hrs} of 8115 (CV 16.8%) h·ng/ml, after administration of three doses with a dosing interval of 8 hours, wherein each of said three doses corresponds to 200 μg/kg, and
   e. within about 80.00% to about 125.00% of an AUC_{0-24 hrs} of 7551 (CV 13.7%) h·ng/ml, after administration of three doses with a dosing interval of 8 hours, wherein each of said three doses corresponds to 250 μg/kg.
110. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 1572 (CV 12.4%) h·ng/ml, after administration of three doses with a dosing interval of 8 hours, wherein each of said three doses corresponds to 50 μg/kg.
111. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 3621 (CV 11.6%) h·ng/ml, after administration of three doses with a dosing interval of 8 hours, wherein each of said three doses corresponds to 100 μg/kg.
112. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of of 5411 (CV 20.6%) h·ng/ml, after administration of three doses with a dosing interval of 8 hours, wherein each of said three doses corresponds to 140 μg/kg.
113. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 8115 (CV 16.8%) h·ng/ml, after administration of three doses with a dosing interval of 8 hours, wherein each of said three doses corresponds to 200 μg/kg.
114. The liquid pharmaceutical composition or liquid pharmaceutical composition for use according to any of the preceding items, wherein the mean AUC_{0-24 hrs} of the liquid pharmaceutical composition is within about 80.00% to about 125.00% of an AUC_{0-24 hrs} of 7551 (CV 13.7%) h·ng/ml, after administration of three doses corresponding to 250 μg/kg with a dosing interval of 8 hours.
115. The liquid pharmaceutical composition or liquid pharmaceutical composition for use according to any of the preceding items, wherein the geometric mean AUC_{0-24 hrs} of the liquid pharmaceutical composition is selected from the group consisting of:
   a. within about 80.00% to about 125.00% of an AUC_{0-24 hrs} of 1562 (CV 12.4%) h·ng/mL, after administration of three doses with a dosing interval of 8 hours, wherein each of said three doses corresponds to 50 μg/kg,
   b. within about 80.00% to about 125.00% of an AUC_{0-24 hrs} of 3001 (CV 11.6%) h·ng/ml, after administration of three doses with a dosing interval of 8 hours, wherein each of said three doses corresponds to 100 μg/kg,
   c. within about 80.00% to about 125.00% of an AUC_{0-24 hrs} of 5310 (CV 20.6%) h·ng/ml, after administration of three doses with a dosing interval of 8 hours, wherein each of said three doses corresponds to 140 μg/kg,
   d. within about 80.00% to about 125.00% of an AUC_{0-24 hrs} of 8119 (CV 16.8%) h·ng/ml, after administration of three doses with a dosing interval of 8 hours, wherein each of said three doses corresponds to 200 μg/kg, and
   e. within about 80.00% to about 125.00% of an AUC_{0-24 hrs} of 7495 (CV 13.7%) h·ng/ml, after administration of three doses with a dosing interval of 8 hours, wherein each of said three doses corresponds to 250 μg/kg.
116. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 1562 (CV 12.4%) h·ng/ml, after administration of three doses with a dosing interval of 8 hours, wherein each of said three doses corresponds to 50 μg/kg.
117. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 3001 (CV 11.6%) h·ng/mL, after administration of three doses with a dosing interval of 8 hours, wherein each of said three doses corresponds to 100 μg/kg.
118. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 5310 (CV 20.6%) h·ng/mL, after administration of three doses with a dosing interval of 8 hours, wherein each of said three doses corresponds to 140 μg/kg.
119. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 8119 (CV 16.8%) h·ng/ml, after administration of three doses with a dosing interval of 8 hours, wherein each of said three doses corresponds to 200 μg/kg.
120. The liquid pharmaceutical composition or liquid pharmaceutical composition for use according to any of the preceding items, wherein the geometric mean AUC_{0-24 hrs} of the liquid pharmaceutical composition is within about 80.00% to about 125.00% of an AUC_{0-24 hrs} of 7495 (CV 13.7%) h·ng/ml, after administration of three doses corresponding to 250 μg/kg with a dosing interval of 8 hours.
121. The liquid pharmaceutical composition or liquid pharmaceutical composition for use according to any of the preceding items, wherein the mean AUC₀₋ₜ₁ of the liquid pharmaceutical composition is selected from the group consisting of:
   a. within about 80.00% to about 125.00% of an AUC₀₋ₜ₁ of 422 (CV 15.8%) h·ng/ml, after administration of three doses with a dosing interval of 8 hours, wherein each of said three doses corresponds to 50 μg/kg,
   b. within about 80.00% to about 125.00% of an AUC₀₋ₜ₁ of 993 (CV 16.2%) h·ng/ml, after administration of three doses with a dosing interval of 8 hours, wherein each of said three doses corresponds to 100 μg/kg,
   c. within about 80.00% to about 125.00% of an AUC₀₋ₜ₁ of 1688 (CV 17.6%) h·ng/mL, after administration of three doses with a dosing interval of 8 hours, wherein each of said three doses corresponds to 140 μg/kg,
   d. within about 80.00% to about 125.00% of an AUC₀₋ₜ₁ of 2520 (CV 17.5%) h·ng/ml, after administration of three doses with a dosing interval of 8 hours, wherein each of said three doses corresponds to 200 μg/kg, and
   e. within about 80.00% to about 125.00% of an AUC₀₋ₜ₁ of 2540 (CV 19.8%) h·ng/mL, after administration of three doses with a dosing interval of 8 hours, wherein each of said three doses corresponds to 250 μg/kg.
122. The liquid pharmaceutical composition or liquid pharmaceutical composition for use according to any of the preceding items, wherein the mean AUC₀₋ₜ₁ of the liquid pharmaceutical composition is within about 80.00% to about 125.00% of an AUC₀₋ₜ₁ of 422 (CV 15.8%) h·ng/ml, after administration of three doses with a dosing interval of 8 hours, wherein each of said three doses corresponds to 50 μg/kg.
123. The liquid pharmaceutical composition or liquid pharmaceutical composition for use according to any of the preceding items, wherein the mean AUC₀₋ₜ₁ of the liquid pharmaceutical composition is within about 80.00% to about 125.00% of an AUC₀₋ₜ₁ of 993 (CV 16.2%) h·ng/ml, after administration of three doses with a dosing interval of 8 hours, wherein each of said three doses corresponds to 100 μg/kg.
124. The liquid pharmaceutical composition or liquid pharmaceutical composition for use according to any of the preceding items, wherein the mean AUC₀₋ₜ₁ of the liquid pharmaceutical composition is within about 80.00% to about 125.00% of an AUC₀₋ₜ₁ of 1688 (CV 17.6%) h·ng/ml, after administration of three doses with a dosing interval of 8 hours, wherein each of said three doses corresponds to 140 μg/kg.
125. The liquid pharmaceutical composition or liquid pharmaceutical composition for use according to any of the preceding items, wherein the mean AUC₀₋ₜ₁ of the liquid pharmaceutical composition is within about 80.00% to about 125.00% of an AUC₀₋ₜ₁ of 2520 (CV 17.5%) h·ng/ml, after administration of three doses with a dosing interval of 8 hours, wherein each of said three doses corresponds to 200 μg/kg.
126. The liquid pharmaceutical composition or liquid pharmaceutical composition for use according to any of the preceding items, wherein the mean AUC₀₋ₜ₁ of the liquid pharmaceutical composition is within about 80.00% to about 125.00% of an AUC₀₋ₜ₁ of 2540 (CV 19.8%) h·ng/ml, after administration of three doses corresponding to 250 μg/kg with a dosing interval of 8 hours.
127. The liquid pharmaceutical composition or liquid pharmaceutical composition for use according to any of the preceding items, wherein the geometric mean AUC₀₋ₜ₁ of the liquid pharmaceutical composition is selected from the group consisting of:
   a. within about 80.00% to about 125.00% of an AUC₀₋ₜ₁ of 417 (CV 15.8%) h·ng/ml, after administration of three doses with a dosing interval of 8 hours, wherein each of said three doses corresponds to 50 μg/kg,
   b. within about 80.00% to about 125.00% of an AUC₀₋ₜ₁ of 982 (CV 16.2%) h·ng/ml, after administration of three doses with a dosing interval of 8
   c. within about 80.00% to about 125.00% of an AUC₀₋ₜ₁ of 1664 (CV 17.6%) h·ng/ml, after administration of three doses with a dosing interval of 8 hours, wherein each of said three doses corresponds to 140 μg/kg,
   d. within about 80.00% to about 125.00% of an AUC₀₋ₜ₁ of 2489 (CV 17.5%) h·ng/mL, after administration of three doses with a dosing interval of 8 hours, wherein each of said three doses corresponds to 200 μg/kg, and
   e. within about 80.00% to about 125.00% of an AUC₀₋ₜ₁ of 2496 (CV 19.8%) h·ng/ml, after administration of three doses with a dosing interval of 8 hours, wherein each of said three doses corresponds to 250 μg/kg.
128. The liquid pharmaceutical composition or liquid pharmaceutical composition for use according to any of the preceding items, wherein the geometric mean AUC₀₋ₜ₁ of the liquid pharmaceutical composition is within about 80.00% to about 125.00% of an AUC₀₋ₜ₁ of 417 (CV 15.8%) h·ng/ml, after administration of three doses with a dosing interval of 8 hours, wherein each of said three doses corresponds to 50 μg/kg.
129. The liquid pharmaceutical composition or liquid pharmaceutical composition for use according to any of the preceding items, wherein the geometric mean AUC₀₋ₜ₁ of the liquid pharmaceutical composition is within about 80.00% to about 125.00% of an AUC₀₋ₜ₁ of 982 (CV 16.2%) h·ng/ml, after administration of three doses with a dosing interval of 8 hours, wherein each of said three doses corresponds to 100 μg/kg.
130. The liquid pharmaceutical composition or liquid pharmaceutical composition for use according to any of the preceding items, wherein the geometric mean AUC₀₋ₜ₁ of the liquid pharmaceutical composition is within about 80.00% to about 125.00% of an AUC₀₋ₜ₁ of 1664 (CV 17.6%) h·ng/ml, after administration of three doses with a dosing interval of 8 hours, wherein each of said three doses corresponds to 140 μg/kg.
131. The liquid pharmaceutical composition or liquid pharmaceutical composition for use according to any of the preceding items, wherein the geometric mean AUC₀₋ₜ₁ of the liquid pharmaceutical composition is within about 80.00% to about 125.00% of an AUC₀₋ₜ₁ of 2489 (CV 17.5%) h·ng/ml, after administration of three doses with a dosing interval of 8 hours, wherein each of said three doses corresponds to 200 μg/kg.
132. The liquid pharmaceutical composition or liquid pharmaceutical composition for use according to any of the preceding items, wherein the geometric mean AUC₀₋ₜ₁ of the liquid pharmaceutical composition is within about 80.00% to about 125.00% of an AUC₀₋ₜ₁ of 2496 (CV 19.8%) h·ng/ml, after administration of three doses corresponding to 250 μg/kg with a dosing interval of 8 hours.
133. The liquid pharmaceutical composition or liquid pharmaceutical composition for use according to any of the preceding items, wherein the mean AUC₀₋ₜ₂ of the liquid pharmaceutical composition is selected from the group consisting of:
   a. within about 80.00% to about 125.00% of an AUC₀₋ₜ₂ of 420 (CV 15.9%) h·ng/ml, after administration of three doses with a dosing interval of 8 hours, wherein each of said three doses corresponds to 50 μg/kg,
   b. within about 80.00% to about 125.00% of an AUC₀₋ₜ₂ of 1128 (CV 12.0%) h·ng/ml, after administration of three doses with a dosing interval of 8 hours, wherein each of said three doses corresponds to 100 μg/kg,
   c. within about 80.00% to about 125.00% of an AUC₀₋ₜ₂ of 1870 (CV 39.0%) h·ng/ml, after administration of three doses with a dosing interval of 8 hours, wherein each of said three doses corresponds to 140 μg/kg,
   d. within about 80.00% to about 125.00% of an AUC₀₋ₜ₂ of 2610 (CV 21.0%) h·ng/ml, after administration of three doses with a dosing interval of 8 hours, wherein each of said three doses corresponds to 200 μg/kg, and
   e. within about 80.00% to about 125.00% of an AUC₀₋ₜ₂ of 2354 (CV 25%) h·ng/mL, after administration of three doses with a dosing interval of 8 hours, wherein each of said three doses corresponds to 250 μg/kg.
134. The liquid pharmaceutical composition or liquid pharmaceutical composition for use according to any of the preceding items, wherein the mean AUC₀₋ₜ₂ of the liquid pharmaceutical composition is within about 80.00% to about 125.00% of an AUC₀₋ₜ₂ of 420 (CV 15.9%) h·ng/ml, after administration of three doses with a dosing interval of 8 hours, wherein each of said three doses corresponds to 50 μg/kg.
135. The liquid pharmaceutical composition or liquid pharmaceutical composition for use according to any of the preceding items, wherein the mean AUC₀₋ₜ₂ of the liquid pharmaceutical composition is within about 80.00% to about 125.00% of an AUC₀₋ₜ₂ of 1128 (CV 12.0%) h·ng/ml, after administration of three doses with a dosing interval of 8 hours, wherein each of said three doses corresponds to 100 μg/kg.
136. The liquid pharmaceutical composition or liquid pharmaceutical composition for use according to any of the preceding items, wherein the mean AUC₀₋ₜ₂ of the liquid pharmaceutical composition is within about 80.00% to about 125.00% of an AUC₀₋ₜ₂ of 1870 (CV 39.0%) h·ng/mL, after administration of three doses with a dosing interval of 8 hours, wherein each of said three doses corresponds to 140 μg/kg.
137. The liquid pharmaceutical composition or liquid pharmaceutical composition for use according to any of the preceding items, wherein the mean AUC₀₋ₜ₂ of the liquid pharmaceutical composition is within about 80.00% to about 125.00% of an AUC₀₋ₜ₂ of 2610 (CV 21.0%) h·ng/ml, after administration of three doses with a dosing interval of 8 hours, wherein each of said three doses corresponds to 200 μg/kg.
138. The liquid pharmaceutical composition or liquid pharmaceutical composition for use according to any of the preceding items, wherein the mean AUC₀₋ₜ₂ of the liquid pharmaceutical composition is within about 80.00% to about 125.00% of an AUC₀₋ₜ₂ of 2354 (CV 25%) h·ng/ml, after administration of three doses corresponding to 250 μg/kg with a dosing interval of 8 hours.
139. The liquid pharmaceutical composition or liquid pharmaceutical composition for use according to any of the preceding items, wherein the geometric mean AUC₀₋ₜ₂ of the liquid pharmaceutical composition is selected from the group consisting of:
   a. within about 80.00% to about 125.00% of an AUC₀₋ₜ₂ of 416 (CV 15.9%) h·ng/ml, after administration of three doses with a dosing interval of 8 hours, wherein each of said three doses corresponds to 50 μg/kg,
   b. within about 80.00% to about 125.00% of an AUC₀₋ₜ₂ of 1121 (CV 12.0%) h·ng/ml, after administration of three doses with a dosing interval of 8 hours, wherein each of said three doses corresponds to 100 μg/kg,
   c. within about 80.00% to about 125.00% of an AUC₀₋ₜ₂ of 1761 (CV 39.0%) h·ng/mL, after administration of three doses with a dosing interval of 8 hours, wherein each of said three doses corresponds to 140 μg/kg,
   d. within about 80.00% to about 125.00% of an AUC₀₋ₜ₂ of 2559 (CV 21.0%) h·ng/mL, after administration of three doses with a dosing interval of 8 hours, wherein each of said three doses corresponds to 200 μg/kg, and
   e. within about 80.00% to about 125.00% of an AUC₀₋ₜ₂ of 2299 (CV 25%) h·ng/mL, after administration of three doses with a dosing interval of 8 hours, wherein each of said three doses corresponds to 250 μg/kg.
140. The liquid pharmaceutical composition or liquid pharmaceutical composition for use according to any of the preceding items, wherein the geometric mean AUC₀₋ₜ₂ of the liquid pharmaceutical composition is within about 80.00% to about 125.00% of an AUC₀₋ₜ₂ of 416 (CV 15.9%) h·ng/ml, after administration of three doses with a dosing interval of 8 hours, wherein each of said three doses corresponds to 50 μg/kg.
141. The liquid pharmaceutical composition or liquid pharmaceutical composition for use according to any of the preceding items, wherein the geometric mean AUC₀₋ₜ₂ of the liquid pharmaceutical composition is within about 80.00% to about 125.00% of an AUC₀₋ₜ₂ of 1121 (CV 12.0%) h·ng/ml, after administration of three doses with a dosing interval of 8 hours, wherein each of said three doses corresponds to 100 μg/kg.
142. The liquid pharmaceutical composition or liquid pharmaceutical composition for use according to any of the preceding items, wherein the geometric mean AUC₀₋ₜ₂ of the liquid pharmaceutical composition is within about 80.00% to about 125.00% of an AUC₀₋ₜ₂ of 1761 (CV 39.0%) h·ng/mL, after administration of three doses with a dosing interval of 8 hours, wherein each of said three doses corresponds to 140 μg/kg.
143. The liquid pharmaceutical composition or liquid pharmaceutical composition for use according to any of the preceding items, wherein the geometric mean AUC₀₋ₜ₂ of the liquid pharmaceutical composition is within about 80.00% to about 125.00% of an AUC₀₋ₜ₂ of 2559 (CV 21.0%) h·ng/ml, after administration of three doses with a dosing interval of 8 hours, wherein each of said three doses corresponds to 200 μg/kg.
144. The liquid pharmaceutical composition or liquid pharmaceutical composition for use according to any of the preceding items, wherein the geometric mean AUC₀₋ₜ₂ of the liquid pharmaceutical composition is within about 80.00% to about 125.00% of an AUC₀₋ₜ₂ of 2299 (CV 25%) h·ng/ml, after administration of three doses corresponding to 250 μg/kg with a dosing interval of 8 hours.
145. The liquid pharmaceutical composition or liquid pharmaceutical composition for use according to any of the preceding items, wherein the mean AUC₀₋ₜ₃ of the liquid pharmaceutical composition is selected from the group consisting of:
   a. within about 80.00% to about 125.00% of an AUC₀₋ₜ₃ of 437 (CV 13.7%) h·ng/ml, after administration of three doses with a dosing interval of 8 hours, wherein each of said three doses corresponds to 50 μg/kg,
   b. within about 80.00% to about 125.00% of an AUC₀₋ₜ₃ of 1091 (CV 11.1%) h·ng/ml, after administration of three doses with a dosing interval of 8 hours, wherein each of said three doses corresponds to 100 μg/kg,
   c. within about 80.00% to about 125.00% of an AUC₀₋ₜ₃ of 1597 (CV 19.2%) h·ng/ml, after administration of three doses with a dosing interval of 8 hours, wherein each of said three doses corresponds to 140 μg/kg,
   d. within about 80.00% to about 125.00% of an AUC₀₋ₜ₃ of 2723 (CV 17.7%) h·ng/mL, after administration of three doses with a dosing interval of 8 hours, wherein each of said three doses corresponds to 200 μg/kg, and
   e. within about 80.00% to about 125.00% of an AUC₀₋ₜ₃ of 2431 (CV 16.1%) h·ng/ml, after administration of three doses with a dosing interval of 8 hours, wherein each of said three doses corresponds to 250 μg/kg.
146. The liquid pharmaceutical composition or liquid pharmaceutical composition for use according to any of the preceding items, wherein the mean AUC₀₋ₜ₃ of the liquid pharmaceutical composition is within about 80.00% to about 125.00% of an AUC₀₋ₜ₃ of 437 (CV 13.7%) h·ng/ml, after administration of three doses with a dosing interval of 8 hours, wherein each of said three doses corresponds to 50 μg/kg.
147. The liquid pharmaceutical composition or liquid pharmaceutical composition for use according to any of the preceding items, wherein the mean AUC₀₋ₜ₃ of the liquid pharmaceutical composition is within about 80.00% to about 125.00% of an AUC₀₋ₜ₃ of 1091 (CV 11.1%) h·ng/mL, after administration of three doses with a dosing interval of 8 hours, wherein each of said three doses corresponds to 100 μg/kg.
148. The liquid pharmaceutical composition or liquid pharmaceutical composition for use according to any of the preceding items, wherein the mean AUC₀₋ₜ₃ of the liquid pharmaceutical composition is within about 80.00% to about 125.00% of an AUC₀₋ₜ₃ of 1597 (CV 19.2%) h·ng/ml, after administration of three doses with a dosing interval of 8 hours, wherein each of said three doses corresponds to 140 μg/kg.
149. The liquid pharmaceutical composition or liquid pharmaceutical composition for use according to any of the preceding items, wherein the mean AUC₀₋ₜ₃ of the liquid pharmaceutical composition is within about 80.00% to about 125.00% of an AUC₀₋ₜ₃ of 2723 (CV 17.7%) h·ng/ml, after administration of three doses with a dosing interval of 8 hours, wherein each of said three doses corresponds to 200 μg/kg.
150. The liquid pharmaceutical composition or liquid pharmaceutical composition for use according to any of the preceding items, wherein the mean AUC₀₋ₜ₃ of the liquid pharmaceutical composition is within about 80.00% to about 125.00% of an AUC₀₋ₜ₃ of 2431 (CV 16.1%) h·ng/ml, after administration of three doses corresponding to 250 μg/kg with a dosing interval of 8 hours.
151. The liquid pharmaceutical composition or liquid pharmaceutical composition for use according to any of the preceding items, wherein the geometric mean AUC₀₋ₜ₃ of the liquid pharmaceutical composition is selected from the group consisting of:
   a. within about 80.00% to about 125.00% of an AUC₀₋ₜ₃ of 433 (CV 13.7%) h·ng/ml, after administration of three doses with a dosing interval of 8 hours, wherein each of said three doses corresponds to 50 μg/kg,
   b. within about 80.00% to about 125.00% of an AUC₀₋ₜ₃ of 1085 (CV 11.1%) h·ng/ml, after administration of three doses with a dosing interval of 8 hours, wherein each of said three doses corresponds to 100 μg/kg,
   c. within about 80.00% to about 125.00% of an AUC₀₋ₜ₃ of 1570 (CV 19.2%) h·ng/mL, after administration of three doses with a dosing interval of 8 hours, wherein each of said three doses corresponds to 140 μg/kg,
   d. within about 80.00% to about 125.00% of an AUC₀₋ₜ₃ of 2687 (CV 17.7%) h·ng/ml, after administration of three doses with a dosing interval of 8 hours, wherein each of said three doses corresponds to 200 μg/kg, and
   e. within about 80.00% to about 125.00% of an AUC₀₋ₜ₃ of 2405 (CV 16.1%) h·ng/ml, after administration of three doses with a dosing interval of 8 hours, wherein each of said three doses corresponds to 250 μg/kg.
152. The liquid pharmaceutical composition or liquid pharmaceutical composition for use according to any of the preceding items, wherein the geometric mean AUC₀₋ₜ₃ of the liquid pharmaceutical composition is within about 80.00% to about 125.00% of an AUC₀₋ₜ₃ of 433 (CV 13.7%) h·ng/ml, after administration of three doses with a dosing interval of 8 hours, wherein each of said three doses corresponds to 50 μg/kg.
153. The liquid pharmaceutical composition or liquid pharmaceutical composition for use according to any of the preceding items, wherein the geometric mean AUC₀₋ₜ₃ of the liquid pharmaceutical composition is within about 80.00% to about 125.00% of an AUC₀₋ₜ₃ of 1085 (CV 11.1%) h·ng/ml, after administration of three doses with a dosing interval of 8 hours, wherein each of said three doses corresponds to 100 μg/kg.
154. The liquid pharmaceutical composition or liquid pharmaceutical composition for use according to any of the preceding items, wherein the geometric mean AUC₀₋ₜ₃ of the liquid pharmaceutical composition is within about 80.00% to about 125.00% of an AUC₀₋ₜ₃ of 1570 (CV 19.2%) h·ng/ml, after administration of three doses with a dosing interval of 8 hours, wherein each of said three doses corresponds to 140 μg/kg.
155. The liquid pharmaceutical composition or liquid pharmaceutical composition for use according to any of the preceding items, wherein the geometric mean AUC₀₋ₜ₃ of the liquid pharmaceutical composition is within about 80.00% to about 125.00% of an AUC₀₋ₜ₃ of 2687 (CV 17.7%) h·ng/ml, after administration of three doses with a dosing interval of 8 hours, wherein each of said three doses corresponds to 200 μg/kg.
156. The liquid pharmaceutical composition or liquid pharmaceutical composition for use according to any of the preceding items, wherein the geometric mean AUC₀₋ₜ₃ of the liquid pharmaceutical composition is within about 80.00% to about 125.00% of an AUC₀₋ₜ₃ of 2405 (CV 16.1%) h·ng/ml, after administration of three doses corresponding to 250 μg/kg with a dosing interval of 8 hours.
157. The liquid pharmaceutical composition or liquid pharmaceutical composition for use according to any of the preceding items, wherein the mean Cₘₐₓ₁ of the liquid pharmaceutical composition is selected from the group consisting of:
   a. within about 80.00% to about 125.00% of a Cₘₐₓ₁ of 406 (CV 12.3%) ng/mL, after administration of three doses with a dosing interval of 8 hours, wherein each of said three doses corresponds to 50 μg/kg,
   b. within about 80.00% to about 125.00% of a Cₘₐₓ₁ of 883 (CV 13.2%) ng/ml, after administration of three doses with a dosing interval of 8 hours, wherein each of said three doses corresponds to 100 μg/kg,
   c. within about 80.00% to about 125.00% of a Cₘₐₓ₁ of 1340 (CV 20.7%) ng/ml, after administration of three doses with a dosing interval of 8 hours, wherein each of said three doses corresponds to 140 μg/kg,
   d. within about 80.00% to about 125.00% of a Cₘₐₓ₁ of 2190 (CV 16.9%) ng/ml, after administration of three doses with a dosing interval of 8 hours, wherein each of said three doses corresponds to 200 μg/kg, and
   e. within about 80.00% to about 125.00% of a Cₘₐₓ₁ of 1960 (CV 26.5%) ng/ml, after administration of three doses with a dosing interval of 8 hours, wherein each of said three doses corresponds to 250 μg/kg.
158. The liquid pharmaceutical composition or liquid pharmaceutical composition for use according to any of the preceding items, wherein the mean Cₘₐₓ₁ of the liquid pharmaceutical composition is within about 80.00% to about 125.00% of a Cₘₐₓ₁ of 406 (CV 12.3%) ng/ml, after administration of three doses with a dosing interval of 8 hours, wherein each of said three doses corresponds to 50 μg/kg.
159. The liquid pharmaceutical composition or liquid pharmaceutical composition for use according to any of the preceding items, wherein the mean Cₘₐₓ₁ of the liquid pharmaceutical composition is within about 80.00% to about 125.00% of a Cₘₐₓ₁ of 883 (CV 13.2%) ng/ml, after administration of three doses with a dosing interval of 8 hours, wherein each of said three doses corresponds to 100 μg/kg.
160. The liquid pharmaceutical composition or liquid pharmaceutical composition for use according to any of the preceding items, wherein the mean Cₘₐₓ₁ of the liquid pharmaceutical composition is within about 80.00% to about 125.00% of a Cₘₐₓ₁ of 1340 (CV 20.7%) ng/mL, after administration of three doses with a dosing interval of 8 hours, wherein each of said three doses corresponds to 140 μg/kg.
161. The liquid pharmaceutical composition or liquid pharmaceutical composition for use according to any of the preceding items, wherein the mean Cₘₐₓ₁ of the liquid pharmaceutical composition is within about 80.00% to about 125.00% of a Cₘₐₓ₁ of 2190 (CV 16.9%) ng/ml, after administration of three doses with a dosing interval of 8 hours, wherein each of said three doses corresponds to 200 μg/kg.
162. The liquid pharmaceutical composition or liquid pharmaceutical composition for use according to any of the preceding items, wherein the mean Cₘₐₓ₁ of the liquid pharmaceutical composition is within about 80.00% to about 125.00% of a Cₘₐₓ₁ of 1960 (CV 26.5%) ng/ml, after administration of three doses corresponding to 250 μg/kg with a dosing interval of 8 hours.
163. The liquid pharmaceutical composition or liquid pharmaceutical composition for use according to any of the preceding items, wherein the geometric mean Cₘₐₓ₁ of the liquid pharmaceutical composition is selected from the group consisting of:
   a. within about 80.00% to about 125.00% of a Cₘₐₓ₁ of 403 (CV 12.3%) ng/ml, after administration of three doses with a dosing interval of 8 hours, wherein each of said three doses corresponds to 50 μg/kg,
   b. within about 80.00% to about 125.00% of a Cₘₐₓ₁ of 877 (CV 13.2%) ng/ml, after administration of three doses with a dosing interval of 8 hours, wherein each of said three doses corresponds to 100 μg/kg,
   c. within about 80.00% to about 125.00% of a Cₘₐₓ₁ of 1310 (CV 20.7%) ng/ml, after administration of three doses with a dosing interval of 8 hours, wherein each of said three doses corresponds to 140 μg/kg,
   d. within about 80.00% to about 125.00% of a Cₘₐₓ₁ of 2170 (CV 16.9%) ng/ml, after administration of three doses with a dosing interval of 8 hours, wherein each of said three doses corresponds to 200 μg/kg, and
   e. within about 80.00% to about 125.00% of a Cₘₐₓ₁ of 1890 (CV 26.5%) ng/ml, after administration of three doses with a dosing interval of 8 hours, wherein each of said three doses corresponds to 250 μg/kg.
164. The liquid pharmaceutical composition or liquid pharmaceutical composition for use according to any of the preceding items, wherein the geometric mean Cₘₐₓ₁ of the liquid pharmaceutical composition is within about 80.00% to about 125.00% of a Cₘₐₓ₁ of 403 (CV 12.3%) ng/ml, after administration of three doses with a dosing interval of 8 hours, wherein each of said three doses corresponds to 50 μg/kg.
165. The liquid pharmaceutical composition or liquid pharmaceutical composition for use according to any of the preceding items, wherein the geometric mean Cₘₐₓ₁ of the liquid pharmaceutical composition is within about 80.00% to about 125.00% of a Cₘₐₓ₁ of 877 (CV 13.2%) ng/ml, after administration of three doses with a dosing interval of 8 hours, wherein each of said three doses corresponds to 100 μg/kg.
166. The liquid pharmaceutical composition or liquid pharmaceutical composition for use according to any of the preceding items, wherein the geometric mean Cₘₐₓ₁ of the liquid pharmaceutical composition is within about 80.00% to about 125.00% of a Cₘₐₓ₁ of 1310 (CV 20.7%) ng/ml, after administration of three doses with a dosing interval of 8 hours, wherein each of said three doses corresponds to 140 μg/kg.
167. The liquid pharmaceutical composition or liquid pharmaceutical composition for use according to any of the preceding items, wherein the geometric mean Cₘₐₓ₁ of the liquid pharmaceutical composition is within about 80.00% to about 125.00% of a Cₘₐₓ₁ of 2170 (CV 16.9%) ng/ml, after administration of three doses with a dosing interval of 8 hours, wherein each of said three doses corresponds to 200 μg/kg.
168. The liquid pharmaceutical composition or liquid pharmaceutical composition for use according to any of the preceding items, wherein the geometric mean Cₘₐₓ₁ of the liquid pharmaceutical composition is within about 80.00% to about 125.00% of a Cₘₐₓ₁ of 1890 (CV 26.5%) ng/mL, after administration of three doses corresponding to 250 μg/kg with a dosing interval of 8 hours.
169. The liquid pharmaceutical composition or liquid pharmaceutical composition for use according to any of the preceding items, wherein the mean Cₘₐₓ₂ of the liquid pharmaceutical composition is selected from the group consisting of:
   a. within about 80.00% to about 125.00% of a Cₘₐₓ₂ of 395 (CV 15.7%) ng/ml, after administration of three doses with a dosing interval of 8 hours, wherein each of said three doses corresponds to 50 μg/kg,
   b. within about 80.00% to about 125.00% of a Cₘₐₓ₂ of 977 (CV 12.4%) ng/ml, after administration of three doses with a dosing interval of 8 hours, wherein each of said three doses corresponds to 100 μg/kg,
   c. within about 80.00% to about 125.00% of a Cₘₐₓ₂ of 1390 (CV 23.6%) ng/ml, after administration of three doses with a dosing interval of 8 hours, wherein each of said three doses corresponds to 140 μg/kg,
   d. within about 80.00% to about 125.00% of a Cₘₐₓ₂ of 2050 (CV 17.6%) ng/ml, after administration of three doses with a dosing interval of 8 hours, wherein each of said three doses corresponds to 200 μg/kg, and
   e. within about 80.00% to about 125.00% of a Cₘₐₓ₂ of 1930 (CV 45.4%) ng/ml, after administration of three doses with a dosing interval of 8 hours, wherein each of said three doses corresponds to 250 μg/kg.
170. The liquid pharmaceutical composition or liquid pharmaceutical composition for use according to any of the preceding items, wherein the mean Cₘₐₓ₂ of the liquid pharmaceutical composition is within about 80.00% to about 125.00% of a Cₘₐₓ₂ of 395 (CV 15.7%) ng/ml, after administration of three doses with a dosing interval of 8 hours, wherein each of said three doses corresponds to 50 μg/kg.
171. The liquid pharmaceutical composition or liquid pharmaceutical composition for use according to any of the preceding items, wherein the mean Cₘₐₓ₂ of the liquid pharmaceutical composition is within about 80.00% to about 125.00% of a Cₘₐₓ₂ of 977 (CV 12.4%) ng/ml, after administration of three doses with a dosing interval of 8 hours, wherein each of said three doses corresponds to 100 μg/kg.
172. The liquid pharmaceutical composition or liquid pharmaceutical composition for use according to any of the preceding items, wherein the mean Cₘₐₓ₂ of the liquid pharmaceutical composition is within about 80.00% to about 125.00% of a Cₘₐₓ₂ of 1390 (CV 23.6%) ng/ml, after administration of three doses with a dosing interval of 8 hours, wherein each of said three doses corresponds to 140 μg/kg.
173. The liquid pharmaceutical composition or liquid pharmaceutical composition for use according to any of the preceding items, wherein the mean Cₘₐₓ₂ of the liquid pharmaceutical composition is within about 80.00% to about 125.00% of a Cₘₐₓ₂ of 2050 (CV 17.6%) ng/mL, after administration of three doses with a dosing interval of 8 hours, wherein each of said three doses corresponds to 200 μg/kg.
174. The liquid pharmaceutical composition or liquid pharmaceutical composition for use according to any of the preceding items, wherein the mean Cₘₐₓ₂ of the liquid pharmaceutical composition is within about 80.00% to about 125.00% of a Cₘₐₓ₂ of 1930 (CV 45.4%) ng/ml, after administration of three doses corresponding to 250 μg/kg with a dosing interval of 8 hours.
175. The liquid pharmaceutical composition or liquid pharmaceutical composition for use according to any of the preceding items, wherein the geometric mean Cₘₐₓ₂ of the liquid pharmaceutical composition is selected from the group consisting of:
   a. within about 80.00% to about 125.00% of a Cₘₐₓ₂ of 391 (CV 15.7%) ng/mL, after administration of three doses with a dosing interval of 8 hours, wherein each of said three doses corresponds to 50 μg/kg,
   b. within about 80.00% to about 125.00% of a Cₘₐₓ₂ of 971 (CV 12.4%) ng/ml, after administration of three doses with a dosing interval of 8 hours, wherein each of said three doses corresponds to 100 μg/kg,
   c. within about 80.00% to about 125.00% of a Cₘₐₓ₂ of 1360 (CV 23.6%) ng/ml, after administration of three doses with a dosing interval of 8 hours, wherein each of said three doses corresponds to 140 μg/kg,
   d. within about 80.00% to about 125.00% of a Cₘₐₓ₂ of 2020 (CV 17.6%) ng/ml, after administration of three doses with a dosing interval of 8 hours, wherein each of said three doses corresponds to 200 μg/kg, and
   e. within about 80.00% to about 125.00% of a Cₘₐₓ₂ of 1800 (CV 45.4%) ng/mL, after administration of three doses with a dosing interval of 8 hours, wherein each of said three doses corresponds to 250 μg/kg.
176. The liquid pharmaceutical composition or liquid pharmaceutical composition for use according to any of the preceding items, wherein the geometric mean Cₘₐₓ₂ of the liquid pharmaceutical composition is within about 80.00% to about 125.00% of a Cₘₐₓ₂ of 391 (CV 15.7%) ng/ml, after administration of three doses with a dosing interval of 8 hours, wherein each of said three doses corresponds to 50 μg/kg.
177. The liquid pharmaceutical composition or liquid pharmaceutical composition for use according to any of the preceding items, wherein the geometric mean Cₘₐₓ₂ of the liquid pharmaceutical composition is within about 80.00% to about 125.00% of a Cₘₐₓ₂ of 971 (CV 12.4%) ng/mL, after administration of three doses with a dosing interval of 8 hours, wherein each of said three doses corresponds to 100 μg/kg.
178. The liquid pharmaceutical composition or liquid pharmaceutical composition for use according to any of the preceding items, wherein the geometric mean Cₘₐₓ₂ of the liquid pharmaceutical composition is within about 80.00% to about 125.00% of a Cₘₐₓ₂ of 1360 (CV 23.6%) ng/ml, after administration of three doses with a dosing interval of 8 hours, wherein each of said three doses corresponds to 140 μg/kg.
179. The liquid pharmaceutical composition or liquid pharmaceutical composition for use according to any of the preceding items, wherein the geometric mean Cₘₐₓ₂ of the liquid pharmaceutical composition is within about 80.00% to about 125.00% of a Cₘₐₓ₂ of 2020 (CV 17.6%) ng/mL, after administration of three doses with a dosing interval of 8 hours, wherein each of said three doses corresponds to 200 μg/kg.
180. The liquid pharmaceutical composition or liquid pharmaceutical composition for use according to any of the preceding items, wherein the geometric mean Cₘₐₓ₂ of the liquid pharmaceutical composition is within about 80.00% to about 125.00% of a Cₘₐₓ₂ of 1800 (CV 45.4%) ng/ml, after administration of three doses corresponding to 250 μg/kg with a dosing interval of 8 hours.
181. The liquid pharmaceutical composition or liquid pharmaceutical composition for use according to any of the preceding items, wherein the mean Cₘₐₓ₃ of the liquid pharmaceutical composition is selected from the group consisting of:
   a. within about 80.00% to about 125.00% of a Cₘₐₓ₃ of 398 (CV 12.3%) ng/ml, after administration of three doses with a dosing interval of 8 hours, wherein each of said three doses corresponds to 50 μg/kg,
   b. within about 80.00% to about 125.00% of a Cₘₐₓ₃ of 970 (CV 17.7%) ng/ml, after administration of three doses with a dosing interval of 8 hours, wherein each of said three doses corresponds to 100 μg/kg,
   c. within about 80.00% to about 125.00% of a Cₘₐₓ₃ of 1240 (CV 18.0%) ng/ml, after administration of three doses with a dosing interval of 8 hours, wherein each of said three doses corresponds to 140 μg/kg,
   d. within about 80.00% to about 125.00% of a Cₘₐₓ₃ of 2170 (CV 20.1%) ng/ml, after administration of three doses with a dosing interval of 8 hours, wherein each of said three doses corresponds to 200 μg/kg, and
   e. within about 80.00% to about 125.00% of a Cₘₐₓ₃ of 1800 (CV 14.7%) ng/ml, after administration of three doses with a dosing interval of 8 hours, wherein each of said three doses corresponds to 250 μg/kg.
182. The liquid pharmaceutical composition or liquid pharmaceutical composition for use according to any of the preceding items, wherein the mean Cₘₐₓ₃ of the liquid pharmaceutical composition is within about 80.00% to about 125.00% of a Cₘₐₓ₃ of 398 (CV 12.3%) ng/ml, after administration of three doses with a dosing interval of 8 hours, wherein each of said three doses corresponds to 50 μg/kg.
183. The liquid pharmaceutical composition or liquid pharmaceutical composition for use according to any of the preceding items, wherein the mean Cₘₐₓ₃ of the liquid pharmaceutical composition is within about 80.00% to about 125.00% of a Cₘₐₓ₃ of 970 (CV 17.7%) ng/ml, after administration of three doses with a dosing interval of 8 hours, wherein each of said three doses corresponds to 100 μg/kg.
184. The liquid pharmaceutical composition or liquid pharmaceutical composition for use according to any of the preceding items, wherein the mean Cₘₐₓ₃ of the liquid pharmaceutical composition is within about 80.00% to about 125.00% of a Cₘₐₓ₃ of 1240 (CV 18.0%) ng/ml, after administration of three doses with a dosing interval of 8 hours, wherein each of said three doses corresponds to 140 μg/kg.
185. The liquid pharmaceutical composition or liquid pharmaceutical composition for use according to any of the preceding items, wherein the mean Cₘₐₓ₃ of the liquid pharmaceutical composition is within about 80.00% to about 125.00% of a Cₘₐₓ₃ of 2170 (CV 20.1%) ng/ml, after administration of three doses with a dosing interval of 8 hours, wherein each of said three doses corresponds to 200 μg/kg.
186. The liquid pharmaceutical composition or liquid pharmaceutical composition for use according to any of the preceding items, wherein the mean Cₘₐₓ₃ of the liquid pharmaceutical composition is within about 80.00% to about 125.00% of a Cₘₐₓ₃ of 1800 (CV 14.7%) ng/ml, after administration of three doses corresponding to 250 μg/kg with a dosing interval of 8 hours.
187. The liquid pharmaceutical composition or liquid pharmaceutical composition for use according to any of the preceding items, wherein the geometric mean Cₘₐₓ₃ of the liquid pharmaceutical composition is selected from the group consisting of:
   a. within about 80.00% to about 125.00% of a Cₘₐₓ₃ of 396 (CV 12.3%) ng/ml, after administration of three doses with a dosing interval of 8 hours, wherein each of said three doses corresponds to 50 μg/kg,
   b. within about 80.00% to about 125.00% of a Cₘₐₓ₃ of 958 (CV 17.7%) ng/ml, after administration of three doses with a dosing interval of 8 hours, wherein each of said three doses corresponds to 100 μg/kg,
   c. within about 80.00% to about 125.00% of a Cₘₐₓ₃ of 1220 (CV 18.0%) ng/mL, after administration of three doses with a dosing interval of 8 hours, wherein each of said three doses corresponds to 140 μg/kg,
   d. within about 80.00% to about 125.00% of a Cₘₐₓ₃ of 2130 (CV 20.1%) ng/ml, after administration of three doses with a dosing interval of 8 hours, wherein each of said three doses corresponds to 200 μg/kg, and
   e. within about 80.00% to about 125.00% of a Cₘₐₓ₃ of 1780 (CV 14.7%) ng/mL, after administration of three doses with a dosing interval of 8 hours, wherein each of said three doses corresponds to 250 μg/kg.
188. The liquid pharmaceutical composition or liquid pharmaceutical composition for use according to any of the preceding items, wherein the geometric mean Cₘₐₓ₃ of the liquid pharmaceutical composition is within about 80.00% to about 125.00% of a Cₘₐₓ₃ of 396 (CV 12.3%) ng/mL, after administration of three doses with a dosing interval of 8 hours, wherein each of said three doses corresponds to 50 μg/kg.
189. The liquid pharmaceutical composition or liquid pharmaceutical composition for use according to any of the preceding items, wherein the geometric mean Cₘₐₓ₃ of the liquid pharmaceutical composition is within about 80.00% to about 125.00% of a Cₘₐₓ₃ of 958 (CV 17.7%) ng/ml, after administration of three doses with a dosing interval of 8 hours, wherein each of said three doses corresponds to 100 μg/kg.
190. The liquid pharmaceutical composition or liquid pharmaceutical composition for use according to any of the preceding items, wherein the geometric mean Cₘₐₓ₃ of the liquid pharmaceutical composition is within about 80.00% to about 125.00% of a Cₘₐₓ₃ of 1220 (CV 18.0%) ng/ml, after administration of three doses with a dosing interval of 8 hours, wherein each of said three doses corresponds to 140 μg/kg.
191. The liquid pharmaceutical composition or liquid pharmaceutical composition for use according to any of the preceding items, wherein the geometric mean Cₘₐₓ₃ of the liquid pharmaceutical composition is within about 80.00% to about 125.00% of a Cₘₐₓ₃ of 2130 (CV 20.1%) ng/ml, after administration of three doses with a dosing interval of 8 hours, wherein each of said three doses corresponds to 200 μg/kg.
192. The liquid pharmaceutical composition or liquid pharmaceutical composition for use according to any of the preceding items, wherein the geometric mean Cₘₐₓ₃ of the liquid pharmaceutical composition is within about 80.00% to about 125.00% of a Cₘₐₓ₃ of 1780 (CV 14.7%) ng/ml, after administration of three doses corresponding to 250 μg/kg with a dosing interval of 8 hours.
193. The liquid pharmaceutical composition or liquid pharmaceutical composition for use according to any of the preceding items, wherein each of said three doses is administered as a 30-minute i.v. infusion to a human subject.
194. The liquid pharmaceutical composition or liquid pharmaceutical composition for use according to any of the preceding items, wherein said AUC_{0-24 hrs}, AUC₀₋ₜ₁, AUC₀₋ₜ₂, AUC₀₋ₜ₃, Cₘₐₓ₁, Cₘₐₓ₂ or Cₘₐₓ₃ is measured after administration of a single dose to a human subject, such as a healthy male Caucasian subject.
195. The liquid pharmaceutical composition or liquid pharmaceutical composition for use according to any of the preceding items, wherein said liquid pharmaceutical composition is diluted prior to administration.
196. The liquid pharmaceutical composition or liquid pharmaceutical composition for use according to item 193, wherein said liquid pharmaceutical composition is diluted based on the subjects body weight and/or to maintain the same volume for infusion.
197. The liquid pharmaceutical composition or liquid pharmaceutical composition for use according to any of the preceding items, wherein said liquid pharmaceutical composition is administered by i.v. infusion with a flow rate of about 0.5 to 10 mL/min., such as of 0.5 to 10 mL/min., such as of 0.5 to 5 mL/min., such as of 0.5 to 4 mL/min., such as of 0.5 to 3 mL/min., such as of 1 to 2 mL/min., such as of about 1.5 mL/min.
198. The liquid pharmaceutical composition or liquid pharmaceutical composition for use according to any of the preceding items, wherein said liquid pharmaceutical composition is administered by i.v. infusion with a flow rate of 1.5 mL/min.
199. The liquid pharmaceutical composition according to any of the preceding items for use in the treatment of a heart disease and/or a cardiovascular disease.
200. The liquid pharmaceutical composition according to any of the preceding items for use in the treatment of a cardiovascular disease selected from the group consisting of coronary artery diseases, such as angina and myocardial infarction, stroke, heart failure, hypertensive heart disease, rheumatic heart disease, cardiomyopathy, abnormal heart rhythms, congenital heart disease, valvular heart disease, carditis, aortic aneurysms, peripheral artery disease, vascular disease, thromboembolic disease, and venous thrombosis.
201. The liquid pharmaceutical composition according to any of the preceding items for use in the treatment of an ischemic condition and/or an inflammatory condition during heart surgery and/or aorta surgery, such as open heart surgery.
202. The liquid pharmaceutical composition according to any of the preceding items for use in the treatment of myocardial ischemic condition and/or reperfusion injury of the heart.
203. The liquid pharmaceutical composition according to any of the preceding items for use in the treatment of coronary artery disease.
204. The liquid pharmaceutical composition according to any of the preceding items for use in the treatment of a coronary artery disease selected from the group consisting of angina and myocardial infarction; stroke, heart failure; peripheral artery disease; thromboembolic disease; and venous thrombosis.
205. The liquid pharmaceutical composition according to any of the preceding items for use in treatment of myocardial infarction.
206. The liquid pharmaceutical composition according to any of the preceding items for use in treatment of acute myocardial infarction.
207. The liquid pharmaceutical composition according to any of the preceding items for use in treatment of an ischemic condition and/or a reperfusion injury selected from the group consisting of myocardial infarction due to myocardial ischemia/reperfusion, myocardial infarction due to myocardial ischemia, myocarditis, sepsis-induced cardiomyopathy, and sepsis-induced myocardial inflammation.
208. The liquid pharmaceutical composition according to any of the preceding items for use in treatment of myocardial infarction due to myocardial ischemia or myocardial infarction due to myocardial ischemia/reperfusion.
209. The liquid pharmaceutical composition according to any of the preceding items for use in the treatment of ST-segment elevation myocardial infarction (STEMI) or non-ST-segment elevation myocardial infarction (NSTEMI).
210. The liquid pharmaceutical composition according to any of the preceding items for use in treatment of ST-elevation myocardial infarction (STEMI).

1. A liquid pharmaceutical composition comprising an Annexin A1 (AnxA1) N-terminal-peptide and at least one pharmaceutically acceptable excipient for use in the treatment of an ischemic condition and/or an inflammatory condition, wherein said liquid pharmaceutical composition is administered to a human subject as a 1-120 minutes intravenous (i.v.) infusion, and wherein said AnxA1 N-terminal-peptide has a terminal elimination half-life (t_{1/2}) of at least 30 minutes after said i.v. infusion.

2. The liquid pharmaceutical composition for use according to claim 1, wherein said liquid pharmaceutical composition is to be administered as a 10-90 minutes i.v. infusion, such as a 10-60 minutes i.v. infusion, such as a 20-40 minutes i.v. infusion, such as a 30 minutes i.v. infusion.

3. The liquid pharmaceutical composition for use according to any of the preceding claims, wherein said AnxA1 N-terminal-peptide has a t_{1/2} of at least 35 minutes, such as of at least 40 minutes, such as of at least 45 minutes, such as of at least 50 minutes, such as of at least 55 minutes, such as of at least 60 minutes, such as of at least 70 minutes, such as of at least 80 minutes, such as of at least 90 minutes, such as of at least 100 minutes, such as of at least 110 minutes, such as of at least 120 minutes after said i.v. infusion.

4. The liquid pharmaceutical composition for use according to any of the preceding claims, wherein said AnxA1 N-terminal-peptide is selected from the group consisting of:
a. AMVSEFLKQAWFIENEEQEYVQTVKSSKGGPGSAVSPYPTFNPSSDV AA (SEQ ID NO: 2; Annexin A1 2-50),
b. AMVSEFLKQAWFIENEEQEYVQTVKSSKGGPGSAVSPYPTFNPSSDV (SEQ ID NO: 3; Annexin A1 2-48),
c. AMVSEFLKQAWFIENEEQEYVQTVKSSKGGPGSAVSPYPTFNPSS (SEQ ID NO: 4; Annexin A1 2-46),
d. AMVSEFLKQAWFIENEEQEYVQTLKSSKGGPGSAVSPYPTFNPSSDV AA (SEQ ID NO: 5; Annexin A1 2-50 V24L),
e. AMVSEFLKQAWFIENEEQEYVQTLKSSKGGPGSAVSPYPTFNPSSDV (SEQ ID NO: 6; Annexin A1 2-48 V24L),
f. AMVSEFLKQAWFIENEEQEYVQTLKSSKGGPGSAVSPYPTFNPSS (SEQ ID NO: 7; Annexin A1 2-46 V24L), and
g. a functional variant of any one of SEQ ID NOs: 2-7 comprising 1 to 6 individual amino acid substitutions.

5. The liquid pharmaceutical composition for use according to any of the preceding claims, wherein said AnxA1 N-terminal-peptide is AMVSEFLKQAWFIENEEQEYVQTLKSSKGGPGSAVSPYPTFNPSSDV (SEQ ID NO: 6; Annexin A1 2-48 V24L), optionally with a C-terminal amidation.

6. The liquid pharmaceutical composition for use according to any of the preceding claims, wherein said liquid pharmaceutical composition comprises 0.5-10 mg/mL of said AnxA1 N-terminal-peptide, such as 1-10 mg/mL, such as 2-9 mg/mL, such as 3-8 mg/mL, such as 4-6 mg/mL.

7. The liquid pharmaceutical composition for use according to any of the preceding claims, wherein said liquid pharmaceutical composition has pH>6.0.

8. The liquid pharmaceutical composition for use according to any of the preceding claims, wherein said liquid pharmaceutical composition has pH≥7.4, such as pH≥7.5, such as pH≥7.6, such as pH≥7.7, such as pH≥7.8, such as pH≥7.9, such as pH≥8.0.

9. The liquid pharmaceutical composition for use according to any of the preceding claims, wherein said liquid pharmaceutical composition has a pH of about 8.2-8.4, such as of about 8.3.

10. The liquid pharmaceutical composition for use according to any of the preceding claims, said liquid pharmaceutical composition comprising:
i) one or more sugar alcohols, and/or
ii) one or more carbohydrates.

11. The liquid pharmaceutical composition for use according to any of the preceding claims, said liquid pharmaceutical composition comprising:
i) a sugar alcohol selected from the group consisting of mannitol, sorbitol, inositol, glycerol, xylitol, propylene glycol, polyethylene glycols (PEGs) and polypropylene/ethylene glycol copolymer, and/or
ii) a carbohydrate selected from the group consisting of sucrose, trehalose, mannose, ribose, maltose, glucose, lactose, galactose and arabinose.

12. The liquid pharmaceutical composition for use according to any of the preceding claims, said liquid pharmaceutical composition comprising:
i) 1-10 mg/mL of said AnxA1 N-terminal-peptide,
ii) 3-5% (w/w) of a sugar alcohol, and
iii) 0.5-1.5% (w/w) of a carbohydrate.

13. The liquid pharmaceutical composition for use according to any of the preceding claims, said liquid pharmaceutical composition comprising:
i) 1-10 mg/ml of said AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer, and
iii) 0.5-1.5% (w/w) of a carbohydrate selected from the group consisting of sucrose, trehalose, mannose, ribose, maltose, glucose, lactose, galactose and arabinose.

14. The liquid pharmaceutical composition for use according to any of the preceding claims, said liquid pharmaceutical composition comprising:
i) 1-10 mg/ml of said AnxA1 N-terminal-peptide,
ii) 3-5% (w/w) of a sugar alcohol,
iii) 0.5-1.5% (w/w) of a carbohydrate,
iv) 0.005-0.015% (w/w) of a surfactant, and
v) 5-15 mM buffer.

15. The liquid pharmaceutical composition for use according to any of the preceding claims, wherein said liquid pharmaceutical composition has a pH of 8.2-8.4, said liquid pharmaceutical composition comprising
i) 1-10 mg/mL of said AnxA1 N-terminal-peptide,
ii) 3-5% (w/w) of a sugar alcohol selected from the group consisting of mannitol, sorbitol, inositol, glycerol, xylitol, propylene glycol, polyethylene glycols (PEGs) and polypropylene/ethylene glycol copolymer,
iii) 0.5-1.5% (w/w) of a carbohydrate selected from the group consisting of sucrose, trehalose, mannose, ribose, maltose, glucose, lactose, galactose and arabinose,
iv) 0.005-0.015% (w/w) of a surfactant selected from the group consisting of Polysorbate-80 and Polysorbate-20, and
v) 5-15 mM buffer selected from the group consisting of Acetate buffer, TRIS, Amine-containing buffers, Amino-acid based buffers such as lysine, hydroxy-lysine histidine and glycylglycine, non-phosphate buffers, bicarbonate buffers, HEPES and borate buffers.

16. The liquid pharmaceutical composition for use according to any of the preceding claims, wherein said liquid pharmaceutical composition comprises sterile water for injection.

17. The liquid pharmaceutical composition for use according to any of the preceding claims, wherein said liquid pharmaceutical composition is to be administered as a once daily i.v. infusion, such as a once daily 30 minutes i.v. infusion.

18. The liquid pharmaceutical composition for use according to any of the preceding claims, wherein said liquid pharmaceutical composition is to be administered as multiple daily i.v. infusions, such as 2, 3, 4 or 5 daily i.v. infusions.

19. The liquid pharmaceutical composition for use according to any of the preceding claims, wherein said liquid pharmaceutical composition is to be administered as three daily i.v. infusions.

20. The liquid pharmaceutical composition for use according to any of the preceding claims, wherein said liquid pharmaceutical composition is to be administered as three daily i.v. infusions with a dosing interval of 4 to 12 hours, such as a dosing interval of 5 to 11 hours, such as a dosing interval of 6 to 10 hours, such as a dosing interval of 7 to 9 hours.

21. The liquid pharmaceutical composition for use according to any of the preceding claims, wherein the once daily i.v. infusion or each of the multiple daily i.v. infusions correspond to administration of a dosage of about 25-250 μg/kg, about 50-250 μg/kg, about 50-225 μg/kg, about 50-200 μg/kg, about 50-175 μg/kg, about 50-150 μg/kg, about 50-125 μg/kg, about 50-100 μg/kg, or about 50-75 μg/kg.

22. The liquid pharmaceutical composition for use according to any of the preceding claims, wherein the once daily i.v. infusion or each of the multiple daily i.v. infusions correspond to administration of a dosage of about 25-250 μg/kg, about 50-250 μg/kg, about 75-250 μg/kg, about 100-250 μg/kg, about 125-250 μg/kg, about 150-250 μg/kg, about 175-250 μg/kg, or about 200-250 μg/kg.

23. The liquid pharmaceutical composition for use according to any of the preceding claims, wherein said liquid pharmaceutical composition is to be administered as 3 daily i.v. infusions.

24. The liquid pharmaceutical composition for use according to any of the preceding claims, wherein said ischemic condition and/or an inflammatory condition is a cardiovascular disease selected from the group consisting of coronary artery diseases, such as angina and myocardial infarction, stroke, heart failure, hypertensive heart disease, rheumatic heart disease, cardiomyopathy, abnormal heart rhythms, congenital heart disease, valvular heart disease, carditis, aortic aneurysms, peripheral artery disease, vascular disease, thromboembolic disease, and venous thrombosis.

25. The liquid pharmaceutical composition for use according to any of the preceding claims, wherein said ischemic condition and/or an inflammatory condition is a myocardial ischemic condition and/or reperfusion injury of the heart.

26. The liquid pharmaceutical composition for use according to any of the preceding claims, wherein said ischemic condition and/or an inflammatory condition is treated during heart surgery and/or aorta surgery, such as open heart surgery.

27. The liquid pharmaceutical composition for use according to any of the preceding claims, wherein said ischemic condition and/or an inflammatory condition is a coronary artery disease.

28. The liquid pharmaceutical composition for use according to any of the preceding claims, wherein the coronary artery disease is selected from the group consisting of angina and myocardial infarction; stroke, heart failure; peripheral artery disease; thromboembolic disease; and venous thrombosis.

29. The liquid pharmaceutical composition for use according to any of the preceding claims, wherein said ischemic condition and/or an inflammatory is myocardial infarction, such as acute myocardial infarction.

30. The liquid pharmaceutical composition for use according to any of the preceding claims, wherein said myocardial infarction is selected from the group consisting of myocardial infarction due to myocardial ischemia/reperfusion, myocardial infarction due to myocardial ischemia, myocarditis, sepsis-induced cardiomyopathy, and sepsis-induced myocardial inflammation.

31. The liquid pharmaceutical composition for use according to any of the preceding claims, wherein said myocardial infarction is myocardial infarction due to myocardial ischemia or myocardial infarction due to myocardial ischemia/reperfusion.

32. The liquid pharmaceutical composition for use according to any of the preceding claims, wherein said myocardial infarction is ST-segment elevation myocardial infarction (STEMI).


SEQUENCE LISTING (9 sequences)

SEQ ID NO: 1 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 49 aa, Homo sapiens
AMVSEFLKQAWFIENEEQEYVQTVKSSKGGPGSAVSPYPTFNPSSDVAA

SEQ ID NO: 3 - protein, 47 aa, Homo sapiens
AMVSEFLKQAWFIENEEQEYVQTVKSSKGGPGSAVSPYPTFNPSSDV

SEQ ID NO: 4 - protein, 45 aa, Homo sapiens
AMVSEFLKQAWFIENEEQEYVQTVKSSKGGPGSAVSPYPTFNPSS

SEQ ID NO: 5 - protein, 49 aa, synthetic construct
  REGION 1..49: note = Mutated Annexin A1 2-50 V24L
  SITE 24: note = MISC_FEATURE - Amino acid V substituted with L
AMVSEFLKQAWFIENEEQEYVQTLKSSKGGPGSAVSPYPTFNPSSDVAA

SEQ ID NO: 6 - protein, 47 aa, synthetic construct
  REGION 1..47: note = Mutated Annexin A1 2-48 V24L
  SITE 24: note = MISC_FEATURE - Amino acid V substituted with L
AMVSEFLKQAWFIENEEQEYVQTLKSSKGGPGSAVSPYPTFNPSSDV

SEQ ID NO: 7 - protein, 45 aa, synthetic construct
  REGION 1..45: note = Mutated Annexin A1 2-46 V24L
  SITE 24: note = MISC_FEATURE - Amino acid V substituted with L
AMVSEFLKQAWFIENEEQEYVQTLKSSKGGPGSAVSPYPTFNPSS

SEQ ID NO: 8 - protein, 25 aa, Homo sapiens
AMVSEFLKQAWFIENEEQEYVQTVK

SEQ ID NO: 9 - protein, 25 aa, synthetic construct
  REGION 1..25: note = Mutated Annexin A1 2-26 V24L
  SITE 24: note = MISC_FEATURE - Amino acid V substituted with L
AMVSEFLKQAWFIENEEQEYVQTLK